SEQUENCE LISTING (45 sequences)

SEQ ID NO: 1 - DNA, 227 bp, Artificial Sequence
  Exemplary CBA promoter
gaggtgagccccacgttctgcttcactctccccatctcccccccctccccacccccaattttgtatttatttattttttaattattttgtgcagcgatgggggcggggcggggcgaggcggagaggtgcggcggcagccaatcggagcggcgcgctccgaaagtttccttttatggcgaggcggcggcggcggcggctctataaaaagcgaagcgcgcggcgggcgg

SEQ ID NO: 2 - DNA, 378 bp, Artificial Sequence
  example CMV enhancer
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattacca

SEQ ID NO: 3 - DNA, 93 bp, Artificial Sequence
  example SV40 intron sequence
ctctaaggtaaatataaaatttttaagtgtataatgtgttaaactactgattctaattgtttctctcttttagattccaacctttggaactga

SEQ ID NO: 4 - DNA, 92 bp, Artificial Sequence
  example MVM intron sequence
aagaggtaagggtttaagggatggttggttggtggggtattaatgtttaattacctggagcacctgcctgaaatcactttttttcaggttgg

SEQ ID NO: 5 - DNA, 82 bp, Artificial Sequence
  example splice donor sequence
gtaagtatcaaggttacaagacaggtttaaggagaccaatagaaactgggcttgtcgagacagagaagactcttgcgtttct

SEQ ID NO: 6 - DNA, 51 bp, Artificial Sequence
  example splice acceptor sequence
gataggcacctattggtcttactgacatccactttgcctttctctccacag

SEQ ID NO: 7 - DNA, 77 bp, Artificial Sequence
  Exemplary recombinogenic region sequence (AK)
gggatttttccgatttcggcctattggttaaaaaatgagctgatttaacaaaaatttaacgcgaattttaacaaaat

SEQ ID NO: 8 - DNA, 208 bp, Artificial Sequence
  Exemplary polyadenylation sequence
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctgggga

SEQ ID NO: 9 - DNA, 130 bp, Artificial Sequence
  example 5′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 10 - DNA, 130 bp, Artificial Sequence
  example 3′ ITR
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 6648 bp, Artificial Sequence
  example MYO7A nucleotide sequence
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggcatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaactacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagtaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgactgtgtgtacgtcatgcccactgtcaccatgccacctcgtgagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaatgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaggtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 12 - DNA, 3108 bp, Artificial Sequence
  example 5′ end portion of a MYO7A transgene
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggcatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaactacgcactcctacacccggcggccactcaaacagccactgctctaccatgacgacgagggtgaccagctg

SEQ ID NO: 13 - DNA, 3540 bp, Artificial Sequence
  example 3′ end portion of a MYO7A transgene
gcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagtaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgactgtgtgtacgtcatgcccactgtcaccatgccacctcgtgagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaatgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaggtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 14 - DNA, 4488 bp, Artificial Sequence
  vector
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatccttatcgggaattcgcccttaagctagcgtgccacctggtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcggggcgaggcggagaggtgcggcggcagccaatcggagcggcgcgctccgaaagtttccttttatggcgaggcggcggcggcggcggctctataaaaagcgaagcgcgcggcgggcggctgcagaagttggtcgtgaggcactgggcagctctaaggtaaatataaaatttttaagtgtataatgtgttaaactactgattctaattgtttctctcttttagattccaacctttggaactgagtgtccaggcggccgccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggcatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaactacgcactcctacacccggcggccactcaaacagccactgctctaccatgacgacgagggtgaccagctggtaagtatcaaggttacaagacaggtttaaggagaccaatagaaactgggcttgtcgagacagagaagactcttgcgtttctgggatttttccgatttcggcctattggttaaaaaatgagctgatttaacaaaaatttaacgcgaattttaacaaaatattaacgtttataatttcaggtggcatctttccaattgaagggcgaattccgatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - DNA, 4383 bp, Artificial Sequence
  vector
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttatatgatcagggatttttccgatttcggcctattggttaaaaaatgagctgatttaacaaaaatttaacgcgaattttaacaaaatattaacgtttataatttcaggtggcatctttcgataggcacctattggtcttactgacatccactttgcctttctctccacaggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagtaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgactgtgtgtacgtcatgcccactgtcaccatgccacctcgtgagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaatgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaggtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtgaccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagcaattcccgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 16 - DNA, 133 bp, Artificial Sequence
  chimeric intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 17 - DNA, 82 bp, Artificial Sequence; identical to SEQ ID NO: 5
  Splicing donor (SD) sequence

SEQ ID NO: 18 - DNA, 82 bp, Artificial Sequence; identical to SEQ ID NO: 5
  splice donor

SEQ ID NO: 19 - DNA, 51 bp, Artificial Sequence; identical to SEQ ID NO: 6
  splice acceptor

SEQ ID NO: 20 - DNA, 77 bp, Artificial Sequence; identical to SEQ ID NO: 7
  AK sequence

SEQ ID NO: 21 - DNA, 4540 bp, Artificial Sequence
  5′CMV-ABCA4-AK
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtaagtatcaaggttacaagacaggtttaaggagaccaatagaaactgggcttgtcgagacagagaagactcttgcgtttctgggatttttccgatttcggcctattggttaaaaaatgagctgatttaacaaaaatttaacgcgaattttaacaaaatattaacgtttataatttcaggtggcatctttccaattgaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 22 - DNA, 4431 bp, Artificial Sequence
  5′CMV-ABCA4-TS
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcacgcgtggtacctctagagtcgacccgggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtaagtatcaaggttacaagacaggtttaaggagaccaatagaaactgggcttgtcgagacagagaagactcttgcgtttctcaattgaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 23 - DNA, 4536 bp, Artificial Sequence
  5′CMV NO INTRON ABCA4 OV
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 24 - DNA, 4167 bp, Artificial Sequence
  5′CMV NO INTRON ABCA4-AK
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtaagtatcaaggttacaagacaggtttaaggagaccaatagaaactgggcttgtcgagacagagaagactcttgcgtttctgggatttttccgatttcggcctattggttaaaaaatgagctgatttaacaaaaatttaacgcgaattttaacaaaatattaacgtttataatttcaggtggcatctttccaattgaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 25 - DNA, 4289 bp, Artificial Sequence
  5′VMD2 ABCA4-AK
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagtaacggccgccagtgtgctggaattcgcccttaataacttaagcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtaagtatcaaggttacaagacaggtttaaggagaccaatagaaactgggcttgtcgagacagagaagactcttgcgtttctgggatttttccgatttcggcctattggttaaaaaatgagctgatttaacaaaaatttaacgcgaattttaacaaaatattaacgtttataatttcaggtggcatctttccaattgaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 26 - DNA, 4436 bp, Artificial Sequence
  5′RHOABCA4-AK
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagcagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccgcctgaattctgcagatatccatcacactggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtaagtatcaaggttacaagacaggtttaaggagaccaatagaaactgggcttgtcgagacagagaagactcttgcgtttctgggatttttccgatttcggcctattggttaaaaaatgagctgatttaacaaaaatttaacgcgaattttaacaaaatattaacgtttataatttcaggtggcatctttccaattgaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 27 - DNA, 4327 bp, Artificial Sequence
  5′RHO ABCA4-TS
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcttcaatattggccattagccatattattcattggttatatagcataaatcaatattggctattggccattgcatacgttgtatctatatcataatatgtacatttatattggctcatgtccaatatgaccgccatgttggcattgattattgactagcagatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccgcctgaattctgcagatatccatcacactggcggccgccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtaagtatcaaggttacaagacaggtttaaggagaccaatagaaactgggcttgtcgagacagagaagactcttgcgtttctcaattgaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 277 bp, Artificial Sequence
  Exemplary CBA promoter
tcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 29 - DNA, 835 bp, Artificial Sequence
  example rhodopsin (Rho) promoter
agatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatcctgagtacctctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtcatccgcctgaattctgcagatatccatcacactg

SEQ ID NO: 30 - DNA, 688 bp, Artificial Sequence
  example vitelliform macular dystrophy 2 (VMD2) promoter
aacggccgccagtgtgctggaattcgcccttaataacttaagcgtcagcatatgcagaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgg

SEQ ID NO: 31 - DNA, 65 bp, Artificial Sequence
  wild-type small T antigen intron
taaatataaaatttttaagtgtataatgtgttaaactactgattctaattgtttgtgtattttag

SEQ ID NO: 32 - DNA, 346 bp, Artificial Sequence
  wild-type large T antigen intron
gtatttgcttcttccttaaatcctggtgttgatgcaatgtactgcaaacaatggcctgagtgtgcaaagaaaatgtctgctaactgcatatgcttgctgtgcttactgaggatgaagcatgaaaatagaaaattatacaggaaagatccacttgtgtgggttgattgctactgcttcgattgctttagaatgtggtttggacttgatctttgtgaaggaaccttacttctgtggtgtgacataattggacaaactacctacagagatttaaagctctaaggtaaatataaaatttttaagtgtataatgtgttaaactactgattctaattgtttgtgtattttag

SEQ ID NO: 33 - DNA, 93 bp, Artificial Sequence
  SV40 intron
ctctaaggtaaatataaaatttttaagtgtataatgtgttaaactactgattctaattgtttgtgtattttagattccaacctatggaactga

SEQ ID NO: 34 - DNA, 77 bp, Artificial Sequence
  Exemplary recombinogenic region sequence (AK)
gggattttgccgatttcggcctattggttaaaaaatgagctgatttaacaaaaatttaacgcgaattttaacaaaat

SEQ ID NO: 35 - DNA, 278 bp, Artificial Sequence
  AP
gtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtacgtgtggaaccgcactgagctcatgcaggcttccctggacccgtctgtgacccatctcatgggtctctttgagcctggagacatgaaatacgagatccaccgagactccacactggacccctccctgatgga

SEQ ID NO: 36 - DNA, 287 bp, Artificial Sequence
  AP1
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 37 - DNA, 288 bp, Artificial Sequence
  AP2
cgcagggcagcctctgtcatctccatcagggaggggtccagtgtggagtctcggtggatctcgtatttcatgtctccaggctcaaagagacccatgagatgggtcacagacgggtccagggaagcctgcatgagctcagtgcggttccacacataccgggcaccctggcgcttcgccagccattcctgcaccagattcttcccgtccagcctggtcccaccttggctgtagtcatctgggtactcagggtctggggttcccatgcgaaacatgtactttcggcctcca

SEQ ID NO: 38 - DNA, 261 bp, Artificial Sequence
  Exemplary polyadenylation sequence
ttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctac

SEQ ID NO: 39 - DNA, 6648 bp, Artificial Sequence
  example MYO7A nucleotide sequence
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcctcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 40 - DNA, 3108 bp, Artificial Sequence
  example 5′ end portion of a MYO7A transgene
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctg

SEQ ID NO: 41 - DNA, 3540 bp, Artificial Sequence
  example 3′ end portion of a MYO7A transgene
gcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcctcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 42 - DNA, 6822 bp, Artificial Sequence
  example ABCA4 nucleotide sequence
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacagaagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 43 - DNA, 2918 bp, Artificial Sequence
  example 5′ end portion of a ABCA4 transgene
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaattcgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccacctt

SEQ ID NO: 44 - DNA, 3904 bp, Artificial Sequence
  example 3′ end portion of a ABCA4 transgene
gtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacagaagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactga

SEQ ID NO: 45 - protein, 130 aa, Artificial Sequence
  Synthetic polypeptide
DMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPV

(19) United States
(12) Patent Application Publication
(10) Pub. No.: US 2022/0389450 A1
(43) Pub. Date: Dec. 8, 2022

(54) VECTOR SYSTEM

(71) Applicant: FONDAZIONE TELETHON, Rome (IT)

(72) Inventors: Alberto AURICCHIO, Rome (IT); Fabio DELL'AQUILA, Rome (IT); Ivana TRAPANI, Rome (IT); Rita FERLA, Rome (IT)

(21) Appl. No.: 17/742,924

(22) Filed: May 12, 2022

(30) Foreign Application Priority Data
May 12, 2021 (EP) 21173687.1

(51) Int. Cl. C12N 15/86; A61P 27/00; C07K 14/47

(52) U.S. Cl. CPC C12N 15/86; A61P 27/00; C07K 14/4716; C12N 2750/14143; C12N 2750/14171; C12N 2800/40; A61K 48/00

(57) ABSTRACT

A vector system for expressing a transgene in a cell, the vector system comprising a first vector and a second vector, wherein: (a) the first vector comprises in a 5′ to 3′ direction: a promoter; an intron; a 5′ end portion of the transgene coding sequence (CDS); a splice donor sequence; and a first recombinogenic region; (b) the second vector comprises in a 5′ to 3′ direction: a second recombinogenic region; a splice acceptor sequence; and a 3′ end portion of the transgene CDS; wherein the 5′ end portion and the 3′ end portion together constitute the transgene CDS, and wherein the intron is not capable of homologous recombination with the splice donor sequence to excise the 5′ end portion of the transgene CDS.

### RELATED APPLICATIONS

This application claims priority to European Patent Application No. 21173687.1, filed May 12, 2021, the entire disclosure of which is hereby incorporated herein by reference.

### FIELD OF THE INVENTION

The present invention relates to vectors and vector systems, in particular vectors and vector systems that enable delivery of large transgenes to a target cell. The invention also relates to uses of the vectors and vector systems in gene therapy.

### BACKGROUND TO THE INVENTION

Gene therapy, for example using adeno-associated viral (AAV) vectors, represents a promising approach for treatment of many inherited retinal degenerations (IRDs). Indeed, a number of years of pre-clinical research and clinical trials for different IRDs have shown the ability of AAV to efficiently deliver therapeutic genes to diseased retinal layers (e.g. photoreceptors (PR) and retinal pigment epithelium (RPE)) and have underlined their excellent safety and efficacy profiles in humans. However, one of the major obstacles in utilising AAV gene therapy vectors is their capacity for packaging transgenes, which may be restricted to a maximum of about 5 kb. This may be a limiting factor for the development of gene replacement therapy for diseases, such as IRDs, which arise due to mutations in genes with a coding sequence (CDS) larger than 5 kb.

Considerable interest has been directed towards the identification of strategies to increase the capacity of AAV. For example, dual AAV vectors that are based on the ability of AAV genomes to concatemerise via intermolecular recombination have been successfully exploited to address this issue. Dual AAV vectors may be generated by splitting a large transgene CDS into separate portions and packaging each in a single normal size (NS; <5 kb) AAV vector. The reconstitution of the full-length transgene CDS may be achieved upon co-infection of the same cell by both dual AAV vectors followed by either: i) inverted terminal repeat (ITR-)-mediated tail-to-head concatemerisation of the two vector genomes followed by splicing (dual AAV trans-splicing, TS) (Duan et al. (2001) Molecular Therapy: the journal of the American Society of Gene Therapy 4: 383-391); ii) homologous recombination between overlapping regions contained in the two vector genomes (dual AAV overlapping, OV) ((Duan et al. (2001) Molecular Therapy: the journal of the American Society of Gene Therapy 4: 383-391)); or iii) a combination of the two (dual AAV hybrid) (Ghosh et al. (2008). Molecular Therapy: the journal of the American Society of Gene Therapy 16: 124-130).

The recombinogenic regions most used in the context of dual AAV hybrid vectors derive from the 872 bp sequence of the middle one-third of the human alkaline phosphatase cDNA that has been shown to confer high levels of dual AAV hybrid vector reconstitution. In addition, a 77 bp sequence from the F1 phage genome (AK) has been found to be highly recombinogenic in vitro and in vivo experiments.

Although studies have highlighted the potential of dual vector systems, such as AAV vector systems, for delivery and reconstitution of large transgenes in a tissue of interest, their translation to clinical use remains a significant unmet need.

### SUMMARY OF THE INVENTION

During development of a dual vector system for the delivery of a large transgene (e.g. Myosin7A, MYO7A), the inventors unexpectedly discovered a consistent contaminant in their preparation of the vector containing the 5′ end portion of the transgene CDS. The inventors analysed the preparations with Southern blots and identified a band corresponding to the expected vector and surprisingly also discovered a smaller size band of about 1.3 kb corresponding to the contaminant.

The inventors then studied the vectors further and identified region of homology between the chimeric promoter intron and the splicing donor (SD) site used in the vector. Further sequencing analysis of purified viral DNA confirmed that a homologous recombination event takes place due to the presence of these regions of homology within the construct, which leads to the deletion of the remaining portion of the intron, the 5′ end portion of the transgene CDS and the SD site while retaining AAV inverted terminal repeats (ITRs), thus supporting vector production.

In one aspect the invention provides a vector system for expressing a transgene in a cell, the vector system comprising a first vector and a second vector, wherein:
(a) the first vector comprises in a 5′ to 3′ direction: an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In another aspect the invention provides a vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In another aspect the invention provides a vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In preferred embodiments, the intron is a simian virus 40 (SV40) intron. The SV40 intron may be a modified SV40 intron.

In some embodiments, the intron is a minute virus mice (MVM) intron.

In some embodiments, the intron comprises a nucleotide sequence with at least 95% sequence identity (e.g. at least 96%, 97%, 98% or 99% sequence identity, or 100% sequence identity) to SEQ ID NO: 3 or 4. In preferred embodiments, the intron comprises a nucleotide sequence with at least 95% sequence identity (e.g. at least 96%, 97%, 98% or 99% sequence identity, or 100% sequence identity) to SEQ ID NO: 3.

In some embodiments, the splice donor sequence comprises a nucleotide sequence with at least 95% sequence identity to SEQ ID NO: 5.

In preferred embodiments, the first recombinogenic region and the second recombinogenic region are the same.

In some embodiments, the first recombinogenic region and the second recombinogenic region are both F1 phage recombinogenic regions or fragments thereof.

In some embodiments, the first recombinogenic region and the second recombinogenic region both comprise a nucleotide sequence with at least 95% sequence identity to SEQ ID NO: 7 or a fragment thereof.

In some embodiments, the first vector and the second vector are viral vectors. The viral vectors may be adeno-associated viral (AAV) vectors, adenoviral vectors, retroviral vectors, lentiviral vectors, herpes simplex viral vectors, picornaviral vectors or alphaviral vectors. In some embodiments, the first vector and the second vector are plasmids. The first and/or second plasmid may, for example, be used to produce the first and/or second viral vector particles (e.g. separately or together in a composition).

In preferred embodiments, the first vector and the second vector are AAV vectors.

In some embodiments, the AAV vectors are of the same serotype (e.g. comprise capsids of the same serotype). In some embodiments, the AAV vectors are of different serotypes (e.g. comprise capsids of different serotypes).

In some embodiments, the first vector and the second vector are selected from the group consisting of AAV2, AAV8, AAV5, AAV7, AAV9, AAV-PhP.B and AAV-PhP.eB. In some embodiments, the first vector and the second vector are selected from the group consisting of hu68 (see, for example, WO 2018/160582), Anc libraries (see, for example, WO 2015/054653 and WO 2017/019994) and AAV2-TT (see, for example, WO 2015/121501).

In some embodiments, the first vector and the second vector are AAV2 vectors. In some embodiments, the first vector and the second vector are AAV8 vectors.

In some embodiments, the first vector and the second vector comprise capsids selected from the group consisting of AAV2, AAV8, AAV5, AAV7, AAV9, AAV-PhP.B and AAV-PhP.eB. In some embodiments, the first vector and the second vector comprise capsids selected from the group consisting of hu68 (see, for example, WO 2018/160582), Anc libraries (see, for example, WO 2015/054653 and WO 2017/019994) and AAV2-TT (see, for example, WO 2015/121501).

In some embodiments, the first vector and the second vector comprise AAV2 capsids. In some embodiments, the first vector and the second vector comprise AAV8 capsids.

In some embodiments, the first vector further comprises a 5′ ITR and a 3′ ITR. In some embodiments, the second vector further comprises a 5′ ITR and a 3′ ITR. In preferred embodiments, the first vector further comprises a 5′ ITR and a 3′ ITR, and the second vector further comprises a 5′ ITR and a 3′ ITR.

In preferred embodiments, the ITRs are AAV ITRs, preferably AAV2 ITRs. In some embodiments, the ITRs are AAV8 ITRs.

In preferred embodiments, the first vector and the second vector are AAV2/8 vectors.

In some embodiments, the ITRs are from the same AAV serotype. In some embodiments, the ITRs are from different AAV serotypes.

In preferred embodiments, the 3′ ITR of the first vector and the 5′ ITR of the second vector are from the same AAV serotype.

In preferred embodiments, the 5′ ITR of the first vector and the 5′ ITR of the second vector are from the same AAV serotype. In preferred embodiments, the 3′ ITR of the first vector and the 3′ ITR of the second vector are from the same AAV serotype.

In preferred embodiments, the 5′ ITR of the first vector and the 5′ ITR of the second vector are AAV2 5′ ITRs, and the 3′ ITR of the first vector and the 3′ ITR of the second vector are AAV2 3′ ITRs.

In some embodiments, the 5′ ITR of the first vector and the 5′ ITR of the second vector are AAV8 5′ ITRs, and the 3′ ITR of the first vector and the 3′ ITR of the second vector are AAV8 3′ ITRs.

In some embodiments, the 5′ ITR of the first vector and the 5′ ITR of the second vector are from different AAV serotypes. In some embodiments, the 3′ ITR of the first vector and the 3′ ITR of the second vector are from different AAV serotypes. In some embodiments, the 5′ ITR of the first vector and the 5′ ITR of the second vector are from different AAV serotypes, and the 3′ ITR of the first vector and the 3′ ITR of the second vector are from different AAV serotypes.

In some embodiments, the 5′ ITR of the first vector and the 3′ ITR of the second vector are from different AAV serotypes.

In some embodiments, the first vector and the second vector are viral vector particles.

In some embodiments, the promoter is a CBA promoter or a fragment thereof.

In some embodiments, the first vector further comprises an enhancer sequence. In preferred embodiments, the enhancer is a CMV enhancer.

In some embodiments, the second vector further comprises a polyadenylation sequence downstream of the 3′ end portion of the transgene CDS. In preferred embodiments, the polyadenylation sequence is a bovine growth hormone (bGH) polyadenylation sequence.

In some embodiments, the transgene is selected from the group consisting of: Myosin 7A (MYO7A), ABCA4, CEP290, CDH23, EYS, USH2a, GPR98 and ALMS1.

In preferred embodiments, the transgene is a Myosin 7A (MYO7A) transgene. In some embodiments, the transgene is an ABCA4 transgene. In some embodiments, the transgene CDS is a wild type sequence. In some embodiments, the transgene CDS is codon optimised (e.g. codon optimised for expression in humans).

In preferred embodiments:
(a) the first vector comprises a nucleotide sequence with at least 95% sequence identity to SEQ ID NO: 14; and/or
(b) the second vector comprises a nucleotide sequence with at least 95% sequence identity to SEQ ID NO: 15.

In particularly preferred embodiments:
(a) the first vector comprises the nucleotide sequence of SEQ ID NO: 14; and/or
(b) the second vector comprises the nucleotide sequence of SEQ ID NO: 15.

In some embodiments, the first vector and second vector are in a 1:1 genome copy ratio.

In another aspect the invention provides a method for expressing a transgene in a cell, comprising transducing or transfecting the cell with the first vector and the second vector as disclosed herein, such that the transgene is expressed in the cell.

In another aspect the invention provides a cell comprising the first vector and the second vector as disclosed herein.

In another aspect the invention provides a cell transduced or transfected with the first vector and the second vector as disclosed herein.

In some embodiments, the cell is a mammalian cell, a human cell, a retinal cell or a non-embryonic stem cell.

In another aspect the invention provides a vector, wherein the vector is the first vector as disclosed herein.

In another aspect the invention provides a vector, wherein the vector is the second vector as disclosed herein.

In another aspect the invention provides a vector comprising in a 5′ to 3′ direction: an intron; a 5′ end portion of a transgene coding sequence (CDS); and a splice donor sequence, wherein the intron is not capable of homologous recombination with the splice donor sequence to excise the 5′ end portion of the transgene CDS.

In another aspect the invention provides a vector comprising in a 5′ to 3′ direction: a promoter; an intron; a 5′ end portion of a transgene coding sequence (CDS); and a splice donor sequence, wherein the intron is not capable of homologous recombination with the splice donor sequence to excise the 5′ end portion of the transgene CDS.

In another aspect the invention provides a vector comprising in a 5′ to 3′ direction: a promoter; an intron; a 5′ end portion of a transgene coding sequence (CDS); a splice donor sequence; and a recombinogenic region, wherein the intron is not capable of homologous recombination with the splice donor sequence to excise the 5′ end portion of the transgene CDS.

In some embodiments, the vector further comprises a 5′ ITR and a 3′ ITR. In preferred embodiments, the ITRs are AAV ITRs, preferably AAV2 ITRs.

In some embodiments, the ITRs are from the same AAV serotype. In some embodiments, the ITRs are from different AAV serotypes.

In another aspect the invention provides a vector comprising in a 5′ to 3′ direction: a splice acceptor sequence; and a 3′ end portion of a transgene coding sequence (CDS).

In another aspect the invention provides a vector comprising in a 5′ to 3′ direction: a recombinogenic region; a splice acceptor sequence; and a 3′ end portion of a transgene coding sequence (CDS).

In some embodiments, the vector comprises a nucleotide sequence with at least 95% sequence identity to SEQ ID NO: 14.

In preferred embodiments, the vector comprises the nucleotide sequence of SEQ ID NO: 14.

In another aspect the invention provides a kit comprising the first vector as disclosed herein and the second vector as disclosed herein.

In another aspect the invention provides a composition comprising the first vector as disclosed herein and the second vector as disclosed herein.

In some embodiments, the first vector and second vector are in a 1:1 genome copy ratio.

In preferred embodiments, the composition is a pharmaceutical composition comprising a pharmaceutically-acceptable carrier, diluent or excipient.

In another aspect the invention provides the vector system, vector, kit or composition of the invention for use in therapy.

In another aspect the invention provides the vector system, vector, kit or composition of the invention for use in treatment of a retinal degeneration. Preferably the retinal degeneration is an inherited retinal degeneration.

In another aspect the invention provides the first vector as disclosed herein for use in therapy, wherein the first vector is administered simultaneously, sequentially or separately in combination with the second vector as disclosed herein.

In another aspect the invention provides the first vector as disclosed herein for use in treatment of a retinal degeneration, wherein the first vector is administered simultaneously, sequentially or separately in combination with the second vector as disclosed herein. Preferably the retinal degeneration is an inherited retinal degeneration.

In another aspect the invention provides the second vector as disclosed herein for use in therapy, wherein the second vector is administered simultaneously, sequentially or separately in combination with the first vector as disclosed herein.

In another aspect the invention provides the second vector as disclosed herein for use in treatment of a retinal degeneration, wherein the second vector is administered simultaneously, sequentially or separately in combination with the first vector as disclosed herein. Preferably the retinal degeneration is an inherited retinal degeneration.

In some embodiments, the use is in treatment or prevention of Usher syndrome, retinitis pigmentosa, Leber congenital amaurosis (LCA), Stargardt disease, Alstrom syndrome or ABCA4-associated diseases.

In another aspect the invention provides the vector system, vector, kit or composition of the invention for use in treatment of Usher syndrome.

In another aspect the invention provides a method of treating or preventing a retinal degeneration comprising administering an effective amount of the vector system, vector, kit or composition of the invention to a subject in need thereof. Preferably the retinal degeneration is an inherited retinal degeneration.

In another aspect the invention provides a method of treating or preventing Usher syndrome comprising administering an effective amount of the vector system, vector, kit or composition of the invention to a subject in need thereof.

### DESCRIPTION OF THE DRAWINGS

FIG. 1. Identification of a contaminant vector.

(A) Southern blot image showing genomes corresponding to the AAV-5′hMYO7A (indicated by top arrow) and to the contaminant vector (indicated by bottom arrow). DNAse: treatment with DNAse for degradation of contaminant external DNA; plasmid: plasmid DNA containing the DNA sequences to generate AAV8-CBA-Chimeric intron-5′hMYO7A; 5′AAV genome-CI: genomic DNA extracted from AAV8-CBA promoter-Chimeric intron-5′hMYO7A; molecular weight marker expressed in kilobases; bp=base pair. CI: chimeric intron. (B) Representation of AAV-5′hMYO7A genome showing the sequence recognised by the southern blot probe. (C) Pairing mechanism between the chimeric promoter's intron and the SD signal (indicated by dotted lines). (D) Representation of the contaminant vector genome showing the sequence recognised by the Southern blot probe. (E) Southern blot analysis of AAV preparations including the following expression cassettes: 1. 5′CMV ABCA4 AK (dual hybrid); 2. 5′CMV ABCA4 TS (dual trans-splicing); 3. 5′CMV NO INTR ABCA4 OV (dual overlapping); 4. 5′CMV NO INTR ABCA4 AK (dual hybrid); 5. 5′VMD2 ABCA4 AK (dual hybrid); 6. 5′RHO ABCA4 AK (dual hybrid); 7. 5′RHO ABCA4 TS (dual trans-splicing). Chimeric intron is present in vectors 1 and 2 and absent in vectors 3 to 7. The dashed box indicates full-length genomes of the expected sizes; the solid box indicates short, truncated genomes.

FIG. 2. In vitro comparison of Chimeric intron, SV40 intron, MVM intron and no intron by EGFP fluorescence.

(A) Representation of the plasmids encoding for EGFP with Chimeric intron, SV40 intron, MVM intron or no intron. (B) Representative microscope fluorescence pictures of transfected HEK293 cells (10× magnification, scale bar 100 μm). CI: Chimeric intron; SV40: Simian virus 40; MVM: minute virus mice.

FIG. 3. In vitro comparison of Chimeric intron, SV40 intron, MVM intron and no intron by Western Blot analysis.

A) Western blot analysis of HEK293 cells 72 hours following infection with dual AAV2-Chimeric intron-hMYO7A, dual AAV2-SV40 intron-hMYO7A, dual AAV2-MVM intron-hMYO7A, dual AAV2-no intron-hMYO7A or no vector. The arrow indicates full-length proteins, 60 μg of proteins were loaded in each lane, for each western blot the molecular marker is reported on the left. Experiment number is reported below each set of samples. Negative control: cells that did not receive dual AAV2-hMYO7A; @MYO7A: western blot with anti-Myosin7A (MYO7A) antibody; @Filamin: western blot with anti-Filamin antibody, used as loading control. SV40 intron: modified simian virus 40 intron; MVM intron: minute virus mice intron.

B) Quantification of hMYO7A levels expressed upon infection with dual AAV2-Chimeric intron-hMYO7A, dual AAV2-SV40 intron-hMYO7A, dual AAV2-MVM intron-hMYO7A or dual AAV2-no intron-hMYO7A in HEK293. Levels of hMYO7A are relative to hMYO7A expressed by dual AAV2-Chimeric intron-hMYO7A. Each filled square represents the value quantified for each sample in the corresponding group. The quantification was performed by Western blot analysis using the anti-MYO7A antibody and measurements of human MYO7A band intensities were normalized to Filamin. Mean value is reported inside the histogram of each group. SV40 intron: modified simian virus 40 intron; MVM intron: minute virus mice intron.

FIG. 4. Comparisons of Chimeric intron, SV40 intron and MVM intron.

Representation of the expression cassettes carried by AAV8-5′hMYO7A. Top: AAV8-5′hMYO7A Chimeric intron; middle: AAV8-5′hMYO7A SV40 intron; bottom: AAV8-5′hMYO7A MVM intron. (B) Southern blot of viral genomes from AAV8-5′hMYO7A Chimeric intron, AAV8-5′hMYO7A SV40 intron and AAV8-5′hMYO7A MVM intron. All samples were treated with DNAse to degrade contaminant external DNA, then viral genome DNA was extracted. 5′AAV genome-CI: viral genome DNA extracted from AAV8-CBA promoter-Chimeric intron-5′hMYO7A; 5′AAV genome-SV40: viral genome DNA extracted from AAV8-CBA promoter-SV40 intron-5′hMYO7A; 5′AAV genome-MVM: viral genome DNA extracted from AAV8-CBA promoter-SV40 intron-5′hMYO7A; molecular weight marker expressed in kilobases; bp=base pair. CI: chimeric intron; SV40: simian virus 40 intron; MVM: minute virus mice intron. (C) Representative western blot analysis of C57BL/6 eyecups 2 weeks following sub-retinal injection of AAV8-5′hMYO7A chimeric intron, AAV8-5′hMYO7A SV40 intron or AAV8-5′hMYO7A MVM intron combined with AAV8-3′hMYO7A-3×FLAG, or excipient. The arrow indicates full-length proteins, 150 μg of proteins were loaded in each lane. Negative control: eyes injected with excipient; @Flag: western blot with anti-flag to recognize full length Myosin7A-3×Flag; @Dysferlin: western blot with anti-Dysferlin antibody, used as loading control. (D) Quantification of hMYO7A levels expressed from AAV8-5′hMYO7A chimeric intron, AAV8-5′hMYO7A SV40 intron or AAV8-5′hMYO7A MVM intron combined with AAV8-3′hMYO7A-3×FLAG in subretinally injected C57BL/6 eyecups. Levels of hMYO7A-3×FLAG are relative to hMYO7A-3×FLAG expressed by AAV8-5′hMYO7A chimeric intron combined with AAV8-3′hMYO7A-3×FLAG. The number (n) of positive eyes for hMYO7A-3×FLAG are depicted below each bar. The quantification was performed by Western blot analysis (Panel C) using the anti-Flag antibody and measurements of hMYO7A-3×FLAG band intensities normalised to Dysferlin. The mean value is depicted above the corresponding bars. Values are represented as mean±standard error of the mean (s.e.m.).

FIG. 5. Dose-dependent improvement of apical melanosome localization and hMYO7A protein reconstitution in shaker mice.

(A) Semi-thin retinal sections stained with Toluidine Blue representative of sh1−/− receiving a subretinal injection of either the solvent, as negative control, or dual AAV8.hMYO7A (doses 1.37E+10, 4.4E+9 or 1.37E+9 total GC/eye) and of sh1+/− receiving a subretinal injection of solvent, as positive control. The scale bar (white bar) is 10 μm. Black arrows point at correctly localized melanosomes. (B) Quantification of melanosome localization in the RPE villi of whole retina sections of sh1 mice three months following subretinal delivery of dual AAV8.hMYO7A. The number of apical melanosomes/100 μm of RPE is reported. Data are represented as single measurement for each eye (dot) and as mean±s.e.m (column). Statistical analyses were made using One-way ANOVA followed by the Tukey post-hoc test. P value vs sh1−/− receiving the Solvent is: ** p<0.01; **** p<0.0001. (C) Representative Western blot analysis of sh1−/− eyecups 5 weeks after subretinal delivery of dual AAV8.hMYO7A at the doses of 1.37E+10, 4.4E+9 or 1.37E+9 total GC/eye. As positive and negative controls, sh1+/− and sh1−/− received a subretinal injection of solvent (same volume than dual AAV), respectively. α-MYO7A, Western blot with anti-Myosin 7A antibody; α-Dysferlin: Western blot with anti-Dysferlin antibody, used as loading control. (D) Quantification of human MYO7A levels expressed in sh1−/− eyecups 5 weeks following subretinal injection of dual AAV8 vectors as percentage (%) of endogenous Myo7a expressed in littermate sh1+/− eyes injected with solvent. The quantification was performed by Western blot analysis using the anti-MYO7A antibody and measurements of MYO7A and Myo7a band intensities normalized to Dysferlin. Data are represented as: mean±s.e.m (the mean value is depicted above the corresponding bars).

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

Vector System

In one aspect the invention provides a vector system for expressing a transgene in a cell, the vector system comprising a first vector and a second vector, wherein:
(a) the first vector comprises in a 5′ to 3′ direction: an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In another aspect the invention provides a vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In another aspect the invention provides a vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In another aspect the invention provides a vector system for expressing a transgene in a cell, the vector system comprising a first vector and a second vector, wherein:
(a) the first vector comprises in a 5′ to 3′ direction: a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS.

In another aspect the invention provides a vector system for expressing a transgene in a cell, the vector system comprising a first vector and a second vector, wherein:
(a) the first vector comprises in a 5′ to 3′ direction: a promoter; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS.

In another aspect the invention provides a vector system for expressing a transgene in a cell, the vector system comprising a first vector and a second vector, wherein:
(a) the first vector comprises in a 5′ to 3′ direction: a promoter; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;

wherein the 5′ end portion and the 3′ end portion together constitute the transgene CDS.

In another aspect the invention provides a combination of vectors for expressing a transgene in a cell, the combination comprising a first vector and a second vector, wherein:
(a) the first vector comprises in a 5′ to 3′ direction: an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In another aspect the invention provides a combination of vectors for expressing a transgene in a cell, the combination comprising a first vector and a second vector, wherein:
(a) the first vector comprises in a 5′ to 3′ direction: a promoter; an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In another aspect the invention provides a combination of vectors for expressing a transgene in a cell, the combination comprising a first vector and a second vector, wherein:
(a) the first vector comprises in a 5′ to 3′ direction: a promoter; an intron; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;

wherein the 5′ end portion and the 3′ end portion together constitute the transgene CDS, and wherein the intron is not capable of homologous recombination with the splice donor sequence to excise the 5′ end portion of the transgene CDS.

In another aspect the invention provides a combination of vectors for expressing a transgene in a cell, the vector system comprising a first vector and a second vector, wherein:
(a) the first vector comprises in a 5′ to 3′ direction: a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS.

In another aspect the invention provides a combination of vectors for expressing a transgene in a cell, the vector system comprising a first vector and a second vector, wherein:
(a) the first vector comprises in a 5′ to 3′ direction: a promoter; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS.

In another aspect the invention provides a combination of vectors for expressing a transgene in a cell, the vector system comprising a first vector and a second vector, wherein:
(a) the first vector comprises in a 5′ to 3′ direction: a promoter; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;

wherein the 5′ end portion and the 3′ end portion together constitute the transgene CDS.

The vector system or combination of vectors of the invention may be used to deliver a transgene to a cell when the transgene is not able to be packaged by a single vector, for example due to size constraints of the vector. For example, AAV vectors may have a capacity for packaging transgenes that is restricted to a maximum of about 5 kb.

When the first vector and second vector are introduced into a cell the transgene CDS may be reconstituted from the 5′ and 3′ end portions. The reconstituted transgene may be expressed in the cell.

For example, reconstitution of the full-length transgene CDS may be achieved upon introduction of both the first and second vector to the same cell by: i) inverted terminal repeat (ITR-)-mediated tail-to-head concatemerisation of the two vector genomes followed by splicing (dual vector trans-splicing, TS); ii) homologous recombination between overlapping regions contained in the two vector genomes (dual vector overlapping, OV); or iii) a combination of the two (dual vector hybrid).

In some embodiments, the portion (e.g. the 5′ and/or 3′ end portion) of the transgene CDS is less than or equal to 10 kb, for example less than or equal to 9.5 kb, 9 kb, 8.5 kb, 8 kb, 7.5 kb, 7 kb, 6.5 kb, 6 kb, 5.5 kb, 5 kb or 4.5 kb. In preferred embodiments, the portion (e.g. the 5′ and/or 3′ end portion) of the transgene CDS is less than or equal to 5 kb.

In some embodiments, the 5′ end portion and the 3′ end portion do not comprise overlapping sequences.

In some embodiments, the transgene CDS is split into the 5′ end portion and the 3′ end portion at a natural exon-exon junction.

The term “not capable of homologous recombination” as used herein may mean that no or substantially no homologous recombination is detectable (e.g. using Southern blot analysis, for example as disclosed in the Examples herein) when the vector is prepared under standard conditions (e.g. in the case of AAV vector particles, transfection of HEK293 cells with plasmids encoding (a) the vector genome; (b) Rep and Cap proteins; and (c) adenoviral helper genes required for AAV production (e.g. E2, E4 and/or VARNA), followed by purification, for example as disclosed in the Examples herein). When the intron is not capable of homologous recombination with the splice donor sequence, excision of the 5′ end portion of the transgene CDS may be minimised or prevented, for example thereby increasing the amount of the transgene CDS that is reconstituted from the 5′ and 3′ end portions when the first and second vectors are introduced into a cell.

In some embodiments, the intron does not comprise a region of at least 20, 30, 40, 50, 60, 70, 80, 90 or 100 contiguous nucleotides having at least 95%, 96%, 97%, 98%, 99% or 100% (preferably 100%) sequence identity to a region of the splice donor sequence. As the intron does not share homology with the splice donor sequence, it is not capable of homologous recombination with that sequence.

In another aspect the invention provides a vector system for expressing a transgene in a cell, the vector system comprising a first vector and a second vector, wherein:
(a) the first vector comprises in a 5′ to 3′ direction: an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron does not comprise a region of at least 20, 30, 40, 50, 60, 70, 80, 90 or 100 contiguous nucleotides having at least 95%, 96%, 97%, 98%, 99% or 100% (preferably 100%) sequence identity to a region of the splice donor sequence.

In another aspect the invention provides a vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron does not comprise a region of at least 20, 30, 40, 50, 60, 70, 80, 90 or 100 contiguous nucleotides having at least 95%, 96%, 97%, 98%, 99% or 100% (preferably 100%) sequence identity to a region of the splice donor sequence.

In another aspect the invention provides a vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;

wherein the 5′ end portion and the 3′ end portion together constitute the transgene CDS, and wherein the intron does not comprise a region of at least 20, 30, 40, 50, 60, 70, 80, 90 or 100 contiguous nucleotides having at least 95%, 96%, 97%, 98%, 99% or 100% (preferably 100%) sequence identity to a region of the splice donor sequence.

In another aspect the invention provides a combination of vectors for expressing a transgene in a cell, the combination comprising a first vector and a second vector, wherein:
(a) the first vector comprises in a 5′ to 3′ direction: an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron does not comprise a region of at least 20, 30, 40, 50, 60, 70, 80, 90 or 100 contiguous nucleotides having at least 95%, 96%, 97%, 98%, 99% or 100% (preferably 100%) sequence identity to a region of the splice donor sequence.

In another aspect the invention provides a combination of vectors for expressing a transgene in a cell, the combination comprising a first vector and a second vector, wherein:
(a) the first vector comprises in a 5′ to 3′ direction: a promoter; an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron does not comprise a region of at least 20, 30, 40, 50, 60, 70, 80, 90 or 100 contiguous nucleotides having at least 95%, 96%, 97%, 98%, 99% or 100% (preferably 100%) sequence identity to a region of the splice donor sequence.

In another aspect the invention provides a combination of vectors for expressing a transgene in a cell, the combination comprising a first vector and a second vector, wherein:
(a) the first vector comprises in a 5′ to 3′ direction: a promoter; an intron; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;

wherein the 5′ end portion and the 3′ end portion together constitute the transgene CDS, and wherein the intron does not comprise a region of at least 20, 30, 40, 50, 60, 70, 80, 90 or 100 contiguous nucleotides having at least 95%, 96%, 97%, 98%, 99% or 100% (preferably 100%) sequence identity to a region of the splice donor sequence.

Promoters and Enhancers

A vector of the invention may comprise a promoter. Suitably, the 5′ end portion of the transgene CDS is operably linked to a promoter. The term “operably linked”, as used herein, means that the parts (e.g. transgene and promoter) are linked together in a manner which enables both to carry out their function substantially unhindered.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. Where the vector is administered for therapy, it is preferred that the promoter is functional in the target cell (e.g. retinal cell).

In some embodiments, the promoter is selected from the group consisting of: cytomegalovirus promoter, Rhodopsin promoter, Rhodopsin kinase promoter, Interphotoreceptor retinoid binding protein promoter, and vitelliform macular dystrophy 2 promoter; or a fragment thereof.

In preferred embodiments, the promoter is a chicken β-actin (CBA) promoter or a fragment thereof.

Exemplary CBA promoter sequences include:

(SEQ ID NO: 1)
GAGGTGAGCCCCACGTTCTGCTTCACTCTCCCCATCTCCCCCCCCTCC
CCACCCCCAATTTTGTATTTATTTATTTTTTAATTATTTTGTGCAGCG
ATGGGGGCGGGGCGGGGCGAGGCGGAGAGGTGCGGCGGCAGCCAATCG
GAGCGGCGCGCTCCGAAAGTTTCCTTTTATGGCGAGGCGGCGGCGGCG
GCGGCTCTATAAAAAGCGAAGCGCGCGGCGGGCGG
(SEQ ID NO: 28)
TCGAGGTGAGCCCCACGTTCTGCTTCACTCTCCCCATCTCCCCCCCCT
CCCCACCCCCAATTTTGTATTTATTTATTTTTTAATTATTTTGTGCAG
CGATGGGGGCGGGGGGGGGGGGGGCGCGCGCCAGGCGGGGCGGGGCGG
GGCGAGGGGCGGGGCGGGGCGAGGCGGAGAGGTGCGGCGGCAGCCAAT
CAGAGCGGCGCGCTCCGAAAGTTTCCTTTTATGGCGAGGCGGCGGCGG
CGGCGGCCCTATAAAAAGCGAAGCGCGCGGCGGGCGG

In some embodiments, the promoter comprises or consists of a nucleic acid sequence that has at least 70%, 80%, 90%, 95%, 96%, 97%, 98%, 99% or 100% nucleotide identity to SEQ ID NO: 1 or a fragment thereof, preferably wherein the promoter substantially retains the natural function of the promoter of SEQ ID NO: 1.

In some embodiments, the promoter comprises or consists of a nucleic acid sequence that has at least 70%, 80%, 90%, 95%, 96%, 97%, 98%, 99% or 100% nucleotide identity to SEQ ID NO: 28 or a fragment thereof, preferably wherein the promoter substantially retains the natural function of the promoter of SEQ ID NO: 28.

In preferred embodiments, the promoter comprises or consists of the nucleic acid sequence of SEQ ID NO: 1 or a fragment thereof.

In preferred embodiments, the first vector comprises a promoter that comprises or consists of the nucleic acid sequence of SEQ ID NO: 1 or a fragment thereof.

An example rhodopsin (Rho) promoter sequence is:

(SEQ ID NO: 29)
AGATCTTCCCCACCTAGCCACCTGGCAAACTGCTCCTTCTCTCAAAGGC
CCAAACATGGCCTCCCAGACTGCAACCCCCAGGCAGTCAGGCCCTGTCT
CCACAACCTCACAGCCACCCTGGACGGAATCTGCTTCTTCCCACATTTG
AGTCCTCCTCAGCCCCTGAGCTCCTCTGGGCAGGGCTGTTTCTTTCCAT
CTTTGTATTCCCAGGGGCCTGCAAATAAATGTTTAATGAACGAACAAGA
GAGTGAATTCCAATTCCATGCAACAAGGATTGGGCTCCTGGGCCCTAGG
CTATGTGTCTGGCACCAGAAACGGAAGCTGCAGGTTGCAGCCCCTGCCC
TCATGGAGCTCCTCCTGTCAGAGGAGTGTGGGGACTGGATGACTCCAGA
GGTAACTTGTGGGGGAACGAACAGGTAAGGGGCTGTGTGACGAGATGAG
AGACTGGGAGAATAAACCAGAAAGTCTCTAGCTGTCCAGAGGACATAGC
ACAGAGGCCCATGGTCCCTATTTCAAACCCAGGCCACCAGACTGAGCTG
GGACCTTGGGACAGACAAGTCATGCAGAAGTTAGGGGACCTTCTCCTCC
CTTTTCCTGGATCCTGAGTACCTCTCCTCCCTGACCTCAGGCTTCCTCC
TAGTGTCACCTTGGCCCCTCTTAGAAGCCAATTAGGCCCTCAGTTTCTG
CAGCGGGGATTAATATGATTATGAACACCCCCAATCTCCCAGATGCTGA
TTCAGCCAGGAGCTTAGGAGGGGGAGGTCACTTTATAAGGGTCTGGGGG
GGTCAGAACCCAGAGTCATCCGCCTGAATTCTGCAGATATCCATGAGAC
TG

In some embodiments, the promoter comprises or consists of a nucleic acid sequence that has at least 70%, 80%, 90%, 95%, 96%, 97%, 98%, 99% or 100% nucleotide identity to SEQ ID NO: 29 or a fragment thereof, preferably wherein the promoter substantially retains the natural function of the promoter of SEQ ID NO: 29.

An example vitelliform macular dystrophy 2 (VMD2) promoter sequence is:

(SEQ ID NO: 30)
AACGGCCGCCAGTGTGCTGGAATTCGCCCTTAATAACTTAAGCGTCAGC
ATATGCAGAATTCTGTCATTTTACTAGGGTGATGAAATTCCCAAGCAAC
ACCATCCTTTTCAGATAAGGGCACTGAGGCTGAGAGAGGAGCTGAAACC
TACCCGGGGTCACCACACACAGGTGGCAAGGCTGGGACCAGAAACCAGG
ACTGTTGACTGCAGCCCGGTATTCATTCTTTCCATAGCCCACAGGGCTG
TCAAAGACCCCAGGGCCTAGTCAGAGGCTCCTCCTTCCTGGAGAGTTCC
TGGCACAGAAGTTGAAGCTCAGCACAGCCCCCTAACCCCCAACTCTCTC
TGCAAGGCCTCAGGGGTCAGAACACTGGTGGAGCAGATCCTTTAGCCTC
TGGATTTTAGGGCCATGGTAGAGGGGGTGTTGCCCTAAATTCCAGCCCT
GGTCTCAGCCCAACACCCTCCAAGAAGAAATTAGAGGGGCCATGGCCAG
GCTGTGCTAGCCGTTGCTTCTGAGCAGATTACAAGAAGGGACTAAGACA
AGGACTCCTTTGTGGAGGTCCTGGCTTAGGGAGTCAAGTGACGGCGGCT
CAGCACTCACGTGGGCAGTGCCAGCCTCTAAGAGTGGGCAGGGGCACTG
GCCACAGAGTCCCAGGGAGTCCCACCAGCCTAGTCGCCAGACCTTCTGT
GG

In some embodiments, the promoter comprises or consists of a nucleic acid sequence that has at least 70%, 80%, 90%, 95%, 96%, 97%, 98%, 99% or 100% nucleotide identity to SEQ ID NO: 30 or a fragment thereof, preferably wherein the promoter substantially retains the natural function of the promoter of SEQ ID NO: 30.

A vector of the invention may comprise an enhancer. Suitably, the 5′ end portion of the transgene CDS is operably linked to an enhancer.

In some embodiments, the enhancer is upstream (i.e. toward the 5′ terminal end of the vector) of the promoter.

An “enhancer” is a region of DNA that can be bound by proteins (activators) to increase the likelihood that transcription of a particular gene will occur. Enhancers are cis-acting. They can be located up to 1 Mbp (1,000,000 bp) away from the gene, upstream or downstream from the start site.

In preferred embodiments, the enhancer is a CMV enhancer.

An example CMV enhancer sequence is:

(SEQ ID NO: 2)
GACATTGATTATTGACTAGTTATTAATAGTAATCAATTACGGGGTCATT
AGTTCATAGCCCATATATGGAGTTCCGCGTTACATAACTTACGGTAAAT
GGCCCGCCTGGCTGACCGCCCAACGACCCCCGCCCATTGACGTCAATAA
TGACGTATGTTCCCATAGTAACGCCAATAGGGACTTTCCATTGACGTCA
ATGGGTGGAGTATTTACGGTAAACTGCCCACTTGGCAGTACATCAAGTG
TATCATATGCCAAGTACGCCCCCTATTGACGTCAATGACGGTAAATGGC
CCGCCTGGCATTATGCCCAGTACATGACCTTATGGGACTTTCCTACTTG
GCAGTACATCTACGTATTAGTCATCGCTATTACCA

In some embodiments, the enhancer comprises or consists of a nucleic acid sequence that has at least 70%, 80%, 90%, 95%, 96%, 97%, 98%, 99% or 100% nucleotide identity to SEQ ID NO: 2 or a fragment thereof, preferably wherein the enhancer substantially retains the natural function of the enhancer of SEQ ID NO: 2.

In preferred embodiments, the enhancer comprises or consists of the nucleic acid sequence of SEQ ID NO: 2 or a fragment thereof.

In preferred embodiments, the first vector comprises an enhancer that comprises or consists of the nucleic acid sequence of SEQ ID NO: 2 or a fragment thereof.

Intron

Introns may be included in a vector to increase transgene expression. Any suitable intron may be used, the selection of which may be readily made by the skilled person, with the proviso that the intron of the first vector is not capable of homologous recombination with the splice donor sequence to excise the 5′ end portion of the transgene CDS.

Exemplary intron sequences include:

TAAATATAAAATTTTTAAGTGTATAATGTGTTAAACTACTGATTCTAAT
TGTTTGTGTATTTTAG
(SEQ ID NO: 31; wild-type small T antigen intron)
GTATTTGCTTCTTCCTTAAATCCTGGTGTTGATGCAATGTACTGCAAAC
AATGGCCTGAGTGTGCAAAGAAAATGTCTGCTAACTGCATATGCTTGCT
GTGCTTACTGAGGATGAAGCATGAAAATAGAAAATTATACAGGAAAGAT
CCACTTGTGTGGGTTGATTGCTACTGCTTCGATTGCTTTAGAATGTGGT
TTGGACTTGATCTTTGTGAAGGAACCTTACTTCTGTGGTGTGACATAAT
TGGACAAACTACCTACAGAGATTTAAAGCTCTAAGGTAAATATAAAATT
TTTAAGTGTATAATGTGTTAAACTACTGATTCTAATTGTTTGTGTATTT
TAG
(SEQ ID NO: 32; wild-type large T antigen intron)
CTCTAAGGTAAATATAAAATTTTTAAGTGTATAATGTGTTAAACTACTG
ATTCTAATTGTTTGTGTATTTTAGATTCCAACCTATGGAACTGA
(SEQ ID NO: 33; SV40 intron, e.g. upstream
sequences are from the large T antigen intron,
and downstream sequences are from SV40 cds)

In some embodiments, the intron comprises or consists of a nucleic acid sequence that has at least 70%, 80%, 90%, 95%, 96%, 97%, 98%, 99% or 100% nucleotide identity to SEQ ID NO: 31, 32 or 33, preferably wherein the intron substantially retains the natural function of the intron of SEQ ID NO: 31, 32 or 33, respectively.

In preferred embodiments, the intron is a simian virus 40 (SV40) intron. The SV40 intron may be a modified SV40 intron (see, for example, Nathwani et al. (2006) Blood 107: 2653-2661).

In some embodiments, the intron is a minute virus mice (MVM) intron.

An example SV40 intron sequence is:

CTCTAAGGTAAATATAAAATTTTTAAGTGTATAATGTGTTAAACTACTG
ATTCTAATTGTTTCTCTCTTTTAGATTCCAACCTTTGGAACTGA
(SEQ ID NO: 3; a modified SV40 intron)

In preferred embodiments, the intron comprises or consists of a nucleic acid sequence that has at least 70%, 80%, 90%, 95%, 96%, 97%, 98%, 99% or 100% nucleotide identity to SEQ ID NO: 3, preferably wherein the intron substantially retains the natural function of the intron of SEQ ID NO: 3.

In some embodiments, the intron comprises or consists of a nucleic acid sequence of SEQ ID NO: 3, or a variant thereof having 4, 3, 2 or 1 nucleotide substitutions, additions or deletions, preferably wherein the intron substantially retains the natural function of the intron of SEQ ID NO: 3.

In preferred embodiments, the intron comprises or consists of the nucleic acid sequence of SEQ ID NO: 3.

In preferred embodiments, the first vector comprises an intron that comprises or consists of the nucleic acid sequence of SEQ ID NO: 3.

An example MVM intron sequence is:

(SEQ ID NO: 4)
AAGAGGTAAGGGTTTAAGGGATGGTTGGTTGGTGGGGTATTAATGTTTA
ATTACCTGGAGCACCTGCCTGAAATCACTTTTTTTCAGGTTGG

In some embodiments, the intron comprises or consists of a nucleic acid sequence that has at least 70%, 80%, 90%, 95%, 96%, 97%, 98%, 99% or 100% nucleotide identity to SEQ ID NO: 4, preferably wherein the intron substantially retains the natural function of the intron of SEQ ID NO: 4.

In some embodiments, the intron comprises or consists of a nucleic acid sequence of SEQ ID NO: 4, or a variant thereof having 4, 3, 2 or 1 nucleotide substitutions, additions or deletions, preferably wherein the intron substantially retains the natural function of the intron of SEQ ID NO: 4.

In preferred embodiments, the intron comprises or consists of the nucleic acid sequence of SEQ ID NO: 4.

In preferred embodiments, the first vector comprises an intron that comprises or consists of the nucleic acid sequence of SEQ ID NO: 4.

In another aspect the invention provides a vector system for expressing a transgene in a cell, the vector system comprising a first vector and a second vector, wherein:
(a) the first vector comprises in a 5′ to 3′ direction: an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;

wherein the 5′ end portion and the 3′ end portion together constitute the transgene CDS, and wherein the intron comprises or consists of a nucleic acid sequence that has at least 70%, 80%, 90%, 95%, 96%, 97%, 98%, 99% or 100% nucleotide identity to SEQ ID NO: 3 or 4, preferably wherein the intron substantially retains the natural function of the intron of SEQ ID NO: 3 or 4, respectively.

In another aspect the invention provides a vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); and a splice donor sequence;
(b) the second vector comprises in a 5′ to 3′ direction: a splice acceptor sequence; and a 3′ end portion of the transgene CDS;
wherein the 5′ end portion and the 3′ end portion together constitute the transgene CDS, and wherein the intron comprises or consists of a nucleic acid sequence that has at least 70%, 80%, 90%, 95%, 96%, 97%, 98%, 99% or 100% nucleotide identity to SEQ ID NO: 3 or 4, preferably wherein the intron substantially retains the natural function of the intron of SEQ ID NO: 3 or 4, respectively.

In another aspect the invention provides a vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;

wherein the 5′ end portion and the 3′ end portion together constitute the transgene CDS, and wherein the intron comprises or consists of a nucleic acid sequence that has at least 70%, 80%, 90%, 95%, 96%, 97%, 98%, 99% or 100% nucleotide identity to SEQ ID NO: 3 or 4, preferably wherein the intron substantially retains the natural function of the intron of SEQ ID NO: 3 or 4, respectively.

Splice Donor and Acceptor

RNA splicing is a form of RNA processing in which a newly made precursor messenger RNA (pre-mRNA) transcript is transformed into a mature messenger RNA (mRNA). During splicing, introns (non-coding regions) are removed and exons (coding regions) are joined together.

Within introns, a donor site (5′ end of the intron), a branch site (near the 3′ end of the intron) and an acceptor site (3′ end of the intron) are required for splicing. The splice donor site includes an almost invariant sequence GU at the 5′ end of the intron, within a larger, less highly conserved region. The splice acceptor site at the 3′ end of the intron terminates the intron with an almost invariant AG sequence. Upstream (5′-ward) from the AG there is a region high in pyrimidines (C and U), or polypyrimidine tract. Further upstream from the polypyrimidine tract is the branchpoint.

A “splice donor sequence” is a nucleotide sequence which can function as a donor site at the 5′ end of an intron. Consensus sequences and frequencies of human splice site regions are describe in Ma et al. (2015) PLoS One 10(6): p.e0130729.

A “splice acceptor sequence” is a nucleotide sequence which can function as an acceptor site at the 3′ end of an intron. Consensus sequences and frequencies of human splice site regions are described in Ma et al. (2015) PLoS One 10(6): p.e0130729.

An example splice donor sequence is:

(SEQ ID NO: 5)
GTAAGTATCAAGGTTACAAGACAGGTTTAAGGAGACCAATAGAAACTGG
GCTTGTCGAGACAGAGAAGACTCTTGCGTTTCT

In some embodiments, the splice donor sequence comprises or consists of a nucleic acid sequence that has at least 70%, 80%, 90%, 95%, 96%, 97%, 98%, 99% or 100% nucleotide identity to SEQ ID NO: 5, preferably wherein the splice donor sequence substantially retains the natural function of the splice donor sequence of SEQ ID NO: 5.

In preferred embodiments, the splice donor sequence comprises or consists of the nucleic acid sequence of SEQ ID NO: 5.

In preferred embodiments, the first vector comprises a splice donor sequence that comprises or consists of the nucleic acid sequence of SEQ ID NO: 5.

An example splice acceptor sequence is:

(SEQ ID NO: 6)
GATAGGCACCTATTGGTCTTACTGACATCCACTTTGCCTTTCTCTCCA
CAG

In some embodiments, the splice acceptor sequence comprises or consists of a nucleic acid sequence that has at least 70%, 80%, 90%, 95%, 96%, 97%, 98%, 99% or 100% nucleotide identity to SEQ ID NO: 6, preferably wherein the splice acceptor sequence substantially retains the natural function of the splice acceptor sequence of SEQ ID NO: 6.

In preferred embodiments, the splice acceptor sequence comprises or consists of the nucleic acid sequence of SEQ ID NO: 6.

In preferred embodiments, the second vector comprises an splice acceptor sequence that comprises or consists of the nucleic acid sequence of SEQ ID NO: 6.

Recombinogenic Region

A recombinogenic region may be added to dual vectors to increase recombination. Preferably, a first recombinogenic region is located downstream of the splice donor sequence in the first vector and a second recombinogenic region is located upstream of the splice acceptor sequence in the second vector.

In preferred embodiments, the first recombinogenic region and the second recombinogenic region are the same.

In some embodiments, the first recombinogenic region and the second recombinogenic region are both F1 phage recombinogenic regions or fragments thereof. In preferred embodiments, the first recombinogenic region and the second recombinogenic region are both AK recombinogenic regions or fragments thereof.

Exemplary recombinogenic region sequences (AK) include:

(SEQ ID NO: 7)
GGGATTTTTCCGATTTCGGCCTATTGGTTAAAAAATGAGCTGATTTAAC
AAAAATTTAACGCGAATTTTAACAAAAT
(SEQ ID NO: 34)
GGGATTTTGCCGATTTCGGCCTATTGGTTAAAAAATGAGCTGATTTAAC
AAAAATTTAACGCGAATTTTAACAAAAT

In some embodiments, the recombinogenic region (e.g. the first recombinogenic region and the second recombinogenic region) comprises or consists of a nucleic acid sequence that has at least 70%, 80%, 90%, 95%, 96%, 97%, 98%, 99% or 100% nucleotide identity to SEQ ID NO: 7 or a fragment thereof, preferably wherein the recombinogenic region substantially retains the natural function of the recombinogenic region of SEQ ID NO: 7.

In some embodiments, the recombinogenic region (e.g. the first recombinogenic region and the second recombinogenic region) comprises or consists of a nucleic acid sequence that has at least 70%, 80%, 90%, 95%, 96%, 97%, 98%, 99% or 100% nucleotide identity to SEQ ID NO: 34 or a fragment thereof, preferably wherein the recombinogenic region substantially retains the natural function of the recombinogenic region of SEQ ID NO: 34.

In preferred embodiments, the recombinogenic region (e.g. the first recombinogenic region and the second recombinogenic region) comprises or consists of the nucleic acid sequence of SEQ ID NO: 7 or a fragment thereof.

In preferred embodiments, the first vector comprises a recombinogenic region that comprises or consists of the nucleic acid sequence of SEQ ID NO: 7 or a fragment thereof.

In preferred embodiments, the second vector comprises a recombinogenic region that comprises or consists of the nucleic acid sequence of SEQ ID NO: 7 or a fragment thereof.

In some embodiments, the first recombinogenic region and the second recombinogenic region are both derived from an alkaline phosphatase gene, such as AP (NM 001632, bp 823-1100, SEQ ID NO: 35); AP1 (XM 005246439.2, bp 1802-1516, SEQ ID NO: 36); AP2 (XM_005246439.2, bp 1225-938, SEQ ID NO: 37).

Exemplary AP recombinogenic region sequences include:

(SEQ ID NO: 35; AP)
GTGATCCTAGGTGGAGGCCGAAAGTACATGTTTCGCATGGGAACCCCAG
ACCCTGAGTACCCAGATGACTACAGCCAAGGTGGGACCAGGCTGGACGG
GAAGAATCTGGTGCAGGAATGGCTGGCGAAGCGCCAGGGTGCCCGGTAC
GTGTGGAACCGCACTGAGCTCATGCAGGCTTCCCTGGACCCGTCTGTGA
CCCATCTCATGGGTCTCTTTGAGCCTGGAGACATGAAATACGAGATCCA
CCGAGACTCCACACTGGACCCCTCCCTGATGGA
(SEQ ID NO: 36; AP1)
CCCCGGGTGCGCGGCGTCGGTGGTGCCGGCGGGGGGCGCCAGGTCGCAG
GCGGTGTAGGGCTCCAGGCAGGCGGCGAAGGCCATGACGTGCGCTATGA
AGGTCTGCTCCTGCACGCCGTGAACCAGGTGCGCCTGCGGGCCGCGCGC
GAACACCGCCACGTCCTCGCCTGCGTGGGTCTCTTCGTCCAGGGGCACT
GCTGACTGCTGCCGATACTCGGGGCTCCCGCTCTCGCTCTCGGTAACAT
CCGGCCGGGCGCCGTCCTTGAGCACATAGCCTGGACCGTTTC
(SEQ ID NO: 37; AP2)
CGCAGGGCAGCCTCTGTCATCTCCATCAGGGAGGGGTCCAGTGTGGAGT
CTCGGTGGATCTCGTATTTCATGTCTCCAGGCTCAAAGAGACCCATGAG
ATGGGTCACAGACGGGTCCAGGGAAGCCTGCATGAGCTCAGTGCGGTTC
CACACATACCGGGCACCCTGGCGCTTCGCCAGCCATTCCTGCACCAGAT
TCTTCCCGTCCAGCCTGGTCCCACCTTGGCTGTAGTCATCTGGGTACTC
AGGGTCTGGGGTTCCCATGCGAAACATGTACTTTCGGCCTCCA

In some embodiments, the recombinogenic region (e.g. the first recombinogenic region and the second recombinogenic region) comprises or consists of a nucleic acid sequence that has at least 70%, 80%, 90%, 95%, 96%, 97%, 98%, 99% or 100% nucleotide identity to SEQ ID NO: 35 or a fragment thereof, preferably wherein the recombinogenic region substantially retains the natural function of the recombinogenic region of SEQ ID NO: 35.

In some embodiments, the recombinogenic region (e.g. the first recombinogenic region and the second recombinogenic region) comprises or consists of a nucleic acid sequence that has at least 70%, 80%, 90%, 95%, 96%, 97%, 98%, 99% or 100% nucleotide identity to SEQ ID NO: 36 or a fragment thereof, preferably wherein the recombinogenic region substantially retains the natural function of the recombinogenic region of SEQ ID NO: 36.

In some embodiments, the recombinogenic region (e.g. the first recombinogenic region and the second recombinogenic region) comprises or consists of a nucleic acid sequence that has at least 70%, 80%, 90%, 95%, 96%, 97%, 98%, 99% or 100% nucleotide identity to SEQ ID NO: 37 or a fragment thereof, preferably wherein the recombinogenic region substantially retains the natural function of the recombinogenic region of SEQ ID NO: 37.

Polyadenylation Sequence

The vector of the present invention may comprise a polyadenylation sequence. Suitably, the transgene is operably linked to a polyadenylation sequence. A polyadenylation sequence may be inserted downstream of the transgene to improve transgene expression.

A polyadenylation sequence typically comprises a polyadenylation signal, a polyadenylation site and a downstream element: the polyadenylation signal comprises the sequence motif recognised by the RNA cleavage complex; the polyadenylation site is the site of cleavage at which a poly-A tails is added to the mRNA; the downstream element is a GT-rich region which usually lies just downstream of the polyadenylation site, which is important for efficient processing.

In some embodiments, the second vector further comprises a polyadenylation sequence downstream of the 3′ end portion of the transgene CDS.

In some embodiments, the polyadenylation sequence is a bovine growth hormone (bGH) polyadenylation sequence or an SV40 polyadenylation sequence.

In preferred embodiments, the polyadenylation sequence is a bovine growth hormone (bGH) polyadenylation sequence.

Exemplary polyadenylation sequences include:

(SEQ ID NO: 8)
CTGTGCCTTCTAGTTGCCAGCCATCTGTTGTTTGCCCCTCCCCCGTGCC
TTCCTTGACCCTGGAAGGTGCCACTCCCACTGTCCTTTCCTAATAAAAT
GAGGAAATTGCATCGCATTGTCTGAGTAGGTGTCATTCTATTCTGGGGG
GTGGGGTGGGGCAGGACAGCAAGGGGGAGGATTGGGAAGACAATAGCAG
GCATGCTGGGGA
(SEQ ID NO: 38)
TTCGAGCAGACATGATAAGATACATTGATGAGTTTGGACAAACCACAAC
TAGAATGCAGTGAAAAAAATGCTTTATTTGTGAAATTTGTGATGCTATT
GCTTTATTTGTAACCATTATAAGCTGCAATAAACAAGTTAACAACAACA
ATTGCATTCATTTTATGTTTCAGGTTCAGGGGGAGATGTGGGAGGTTTT
TTAAAGCAAGTAAAACCTCTACAAATGTGGTAAAATCGATAAGGATCTT
CCTAGAGCATGGCTAC

In some embodiments, the polyadenylation sequence comprises or consists of a nucleic acid sequence that has at least 70%, 80%, 90%, 95%, 96%, 97%, 98%, 99% or 100% nucleotide identity to SEQ ID NO: 8, preferably wherein the polyadenylation sequence substantially retains the natural function of the polyadenylation sequence of SEQ ID NO: 8.

In some embodiments, the polyadenylation sequence comprises or consists of a nucleic acid sequence that has at least 70%, 80%, 90%, 95%, 96%, 97%, 98%, 99% or 100% nucleotide identity to SEQ ID NO: 38, preferably wherein the polyadenylation sequence substantially retains the natural function of the polyadenylation sequence of SEQ ID NO: 38.

In preferred embodiments, the polyadenylation sequence comprises or consists of the nucleic acid sequence of SEQ ID NO: 8.

In preferred embodiments, the second vector comprises a polyadenylation sequence that comprises or consists of the nucleic acid sequence of SEQ ID NO: 8.

Vector

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid or facilitating the expression of the protein encoded by a segment of nucleic acid. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

Vectors may be introduced into cells using a variety of techniques known in the art, such as transfection, transformation and transduction. Several such techniques are known in the art, for example infection with recombinant viral vectors, such as retroviral, lentiviral (e.g. integration-defective lentiviral), adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Viral Vectors

In preferred embodiments, the vector is a viral vector, for example comprises a viral (preferably AAV) vector genome. The viral vector may be in the form of a viral vector particle.

The viral vector may be an adeno-associated viral (AAV) vector, adenoviral vector, retroviral vector, lentiviral vector, herpes simplex viral vector, picornaviral vector or alphaviral vector.

In preferred embodiments, the first vector and the second vector are AAV vectors. The AAV vectors may be in the form of AAV vector particles.

Adeno-Associated Viral Vector

The AAV vector or AAV vector particle may comprise an AAV genome or a fragment or derivative thereof. An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

AAVs occurring in nature may be classified according to various biological systems. The AAV genome may be from any naturally derived serotype, isolate or clade of AAV.

AAV may be referred to in terms of their serotype. A serotype corresponds to a variant subspecies of AAV which, owing to its profile of expression of capsid surface antigens, has a distinctive reactivity which can be used to distinguish it from other variant subspecies. Typically, an AAV vector particle having a particular AAV serotype does not efficiently cross-react with neutralising antibodies specific for any other AAV serotype. AAV serotypes include AAV1, AAV2, AAV3, AAV4, AAV5, AAV6, AAV7, AAV8, AAV9, AAV10, AAV11, AAV-PhP.B and AAV-PhP.eB.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. Suitably, one or more ITR sequences flank the transgene or portions thereof.

The AAV genome may also comprise packaging genes, such as rep and/or cap genes which encode packaging functions for an AAV particle. A promoter may be operably linked to each of the packaging genes. Specific examples of such promoters include the p5, p19 and p40 promoters. For example, the p5 and p19 promoters are generally used to express the rep gene, while the p40 promoter is generally used to express the cap gene. The rep gene encodes one or more of the proteins Rep78, Rep68, Rep52 and Rep40 or variants thereof.

The cap gene encodes one or more capsid proteins such as VP1, VP2 and VP3 or variants thereof.

The AAV genome may be the full genome of a naturally occurring AAV. For example, a vector comprising a full AAV genome may be used to prepare an AAV vector or vector particle.

Suitably, the AAV genome is derivatised for the purpose of administration to patients. Such derivatisation is standard in the art and the invention encompasses the use of any known derivative of an AAV genome, and derivatives which could be generated by applying techniques known in the art. The AAV genome may be a derivative of any naturally occurring AAV. Suitably, the AAV genome is a derivative of AAV1, AAV2, AAV3, AAV4, AAV5, AAV6, AAV7, AAV8, AAV9, AAV10, or AAV11.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This may reduce the risk of recombination of the vector with wild-type virus, and avoid triggering a cellular immune response by the presence of viral gene proteins in the target cell.

Typically, a derivative will include at least one inverted terminal repeat sequence (ITR), optionally more than one ITR, such as two ITRs or more. One or more of the ITRs may be derived from AAV genomes having different serotypes, or may be a chimeric or mutant ITR. A suitable mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The AAV genome may comprise one or more ITR sequences from any naturally derived serotype, isolate or clade of AAV or a variant thereof. The AAV genome may comprise at least one, such as two, AAV1, AAV2, AAV3, AAV4, AAV5, AAV6, AAV7, AAV8, AAV9, AAV10, or AAV11 ITRs, or variants thereof.

The one or more ITRs may flank the transgene or portion thereof at either end. The inclusion of one or more ITRs is can aid concatemer formation of the AAV vector in the nucleus of a host cell, for example following the conversion of single-stranded vector DNA into double-stranded DNA by the action of host cell DNA polymerases. The formation of such episomal concatemers protects the AAV vector during the life of the host cell, thereby allowing for prolonged expression of the transgene in vivo.

Suitably, ITR elements will be the only sequences retained from the native AAV genome in the derivative. Suitably, a derivative may not include the rep and/or cap genes of the native genome and any other sequences of the native genome. This may reduce the possibility of integration of the vector into the host cell genome. Additionally, reducing the size of the AAV genome allows for increased flexibility in incorporating other sequence elements (such as regulatory elements) within the vector in addition to the transgene or portion thereof.

The following portions could therefore be removed in a derivative of the invention: one inverted terminal repeat (ITR) sequence, the replication (rep) and capsid (cap) genes. However, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the AAV vector may be tolerated in a therapeutic setting.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV vector particle may be encapsidated by capsid proteins. Suitably, the AAV vector particles may be transcapsidated forms wherein an AAV genome or derivative having an ITR of one serotype is packaged in the capsid of a different serotype. The AAV vector particle also includes mosaic forms wherein a mixture of unmodified capsid proteins from two or more different serotypes makes up the viral capsid. The AAV vector particle also includes chemically modified forms bearing ligands adsorbed to the capsid surface. For example, such ligands may include antibodies for targeting a particular cell surface receptor.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). The AAV vector may be in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery and/or decreased immunogenicity (humoral or cellular) compared to an AAV vector comprising a naturally occurring AAV genome. Increased efficiency of gene delivery, for example, may be effected by improved receptor or co-receptor binding at the cell surface, improved internalisation, improved trafficking within the cell and into the nucleus, improved uncoating of the viral particle and improved conversion of a single-stranded genome to double-stranded form.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence. The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle.

The capsid protein may be an artificial or mutant capsid protein. The term “artificial capsid” as used herein means that the capsid particle comprises an amino acid sequence which does not occur in nature or which comprises an amino acid sequence which has been engineered (e.g. modified) from a naturally occurring capsid amino acid sequence. In other words the artificial capsid protein comprises a mutation or a variation in the amino acid sequence compared to the sequence of the parent capsid from which it is derived where the artificial capsid amino acid sequence and the parent capsid amino acid sequences are aligned.

In some embodiments, the first vector and the second vector are selected from the group consisting of hu68 (see, for example, WO 2018/160582), Anc libraries (see, for example, WO 2015/054653 and WO 2017/019994) and AAV2-TT (see, for example, WO 2015/121501).

An example 5′ ITR sequence is:

(SEQ ID NO: 9)
CTGCGCGCTCGCTCGCTCACTGAGGCCGCCCGGGCAAAGCCCGGGCGTC
GGGCGACCTTTGGTCGCCCGGCCTCAGTGAGCGAGCGAGCGCGCAGAGA
GGGAGTGGCCAACTCCATCACTAGGGGTTCCT

In some embodiments, the 5′ ITR comprises or consists of a nucleic acid sequence that has at least 70%, 80%, 90%, 95%, 96%, 97%, 98%, 99% or 100% nucleotide identity to SEQ ID NO: 9, preferably wherein the 5′ ITR substantially retains the natural function of the 5′ ITR of SEQ ID NO: 9.

In preferred embodiments, the 5′ ITR comprises or consists of the nucleic acid sequence of SEQ ID NO: 9.

In preferred embodiments, the first vector and the second vector comprise a 5′ ITR that comprises or consists of the nucleic acid sequence of SEQ ID NO: 9.

An example 3′ ITR sequence is:

(SEQ ID NO: 10)
AGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCC
CGGGCGGCCTCAGTGAGCGAGCGAGCGCGCAG

In some embodiments, the 3′ ITR comprises or consists of a nucleic acid sequence that has at least 70%, 80%, 90%, 95%, 96%, 97%, 98%, 99% or 100% nucleotide identity to SEQ ID NO: 10, preferably wherein the 3′ ITR substantially retains the natural function of the 3′ ITR of SEQ ID NO: 10.

In preferred embodiments, the 3′ ITR comprises or consists of the nucleic acid sequence of SEQ ID NO: 10.

In preferred embodiments, the first vector and the second vector comprise a 3′ ITR that comprises or consists of the nucleic acid sequence of SEQ ID NO: 10.

Transgene

In some embodiments, the transgene is selected from the group consisting of: Myosin 7A (MYO7A), ABCA4, CEP290, CDH23, EYS, USH2a, GPR98 and ALMS1.

In preferred embodiments, the transgene is a Myosin 7A (MYO7A) transgene.

An example MYO7A nucleotide sequence is:

(SEQ ID NO: 11)
ATGGTGATTCTTCAGCAGGGGGACCATGTGTGGATGGACCTGAGATTGGGGCAGGAGTTCGA
CGTGCCCATCGGGGCGGTGGTGAAGCTCTGCGACTCTGGGCAGGTCCAGGTGGTGGATGATG
AAGACAATGAACACTGGATCTCTCCGCAGAACGCAACGCACATCAAGCCTATGCACCCCACG
TCGGTCCACGGCGTGGAGGACATGATCCGCCTGGGGGACCTCAACGAGGCGGGCATCTTGCG
CAACCTGCTTATCCGCTACCGGGACCACCTCATCTACACGTATACGGGCTCCATCCTGGTGG
CTGTGAACCCCTACCAGCTGCTCTCCATCTACTCGCCAGAGCACATCCGCCAGTATACCAAC
AAGAAGATTGGGGAGATGCCCCCCCACATCTTTGCCATTGCTGACAACTGCTACTTCAACAT
GAAACGCAACAGCCGAGACCAGTGCTGCATCATCAGTGGGGAATCTGGGGCCGGGAAGACGG
AGAGCACAAAGCTGATCCTGCAGTTCCTGGCAGCCATCAGTGGGCAGCACTCGTGGATTGAG
CAGCAGGTCTTGGAGGCCACCCCCATTCTGGAAGCATTTGGGAATGCCAAGACCATCCGCAA
TGACAACTCAAGCCGTTTCGGAAAGTACATCGACATCCACTTCAACAAGCGGGGCGCCATCG
AGGGCGCGAAGATTGAGCAGTACCTGCTGGAAAAGTCACGTGTCTGTCGCCAGGCCCTGGAT
GAAAGGAACTACCACGTGTTCTACTGCATGCTGGAGGGCATGAGTGAGGATCAGAAGAAGAA
GCTGGGCTTGGGCCAGGCCTCTGACTACAACTACTTGGCCATGGGTAACTGCATAACCTGTG
AGGGCCGGGTGGACAGCCAGGAGTACGCCAACATCCGCTCCGCCATGAAGGTGCTCATGTTC
ACTGACACCGAGAACTGGGAGATCTCGAAGCTCCTGGCTGCCATCCTGCACCTGGGCAACCT
GCAGTATGAGGCACGCACATTTGAAAACCTGGATGCCTGTGAGGTTCTCTTCTCCCCATCGC
TGGCCACAGCTGCATCCCTGCTTGAGGTGAACCCCCCAGACCTGATGAGCTGCCTGACTAGC
CGCACCCTCATCACCCGCGGGGAGACGGTGTCCACCCCACTGAGCAGGGAACAGGCACTGGA
CGTGCGCGACGCCTTCGTAAAGGGGATCTACGGGCGGCTGTTCGTGTGGATTGTGGACAAGA
TCAACGCAGCAATTTACAAGCCTCCCTCCCAGGATGTGAAGAACTCTCGCAGGTCCATCGGC
CTCCTGGACATCTTTGGGTTTGAGAACTTTGCTGTGAACAGCTTTGAGCAGCTCTGCATCAA
CTTCGCCAATGAGCACCTGCAGCAGTTCTTTGTGCGGCACGTGTTCAAGCTGGAGCAGGAGG
AATATGACCTGGAGAGCATTGACTGGCTGCACATCGAGTTCACTGACAACCAGGATGCCCTG
GAGATGATTGCCAACAAGCCCATGAACATCATCTCCCTCATCGATGAGGAGAGCAAGTTCCC
CAAGGGCACAGACACCACCATGTTACACAAGCTGAACTCCCAGCACAAGCTCAACGCCAACT
ACATCCCCCCCAAGAACAACCATGAGACCCAGTTTGGCATCAACCATTTTGCAGGCATCGTC
TACTATGAGACCCAAGGCTTCCTGGAGAAGAACCGAGACACCCTGCATGGGGACATTATCCA
GCTGGTCCACTCCTCCAGGAACAAGTTCATCAAGCAGATCTTCCAGGCCGATGTCGCCATGG
GCGCCGAGACCAGGAAGCGCTCGCCCACACTTAGCAGCCAGTTCAAGCGGTCACTGGAGCTG
CTGATGCGCACGCTGGGTGCCTGCCAGCCCTTCTTTGTGCGATGCATCAAGCCCAATGAGTT
CAAGAAGCCCATGCTGTTCGACCGGCACCTGTGCGTGCGCCAGCTGCGGTACTCAGGAATGA
TGGAGACCATCCGAATCCGCCGAGCTGGCTACCCCATCCGCTACAGCTTCGTAGAGTTTGTG
GAGCGGTACCGTGTGCTGCTGCCAGGTGTGAAGCCGGCCTACAAGCAGGGCGACCTCCGCGG
GACTTGCCAGCGCATGGCTGAGGCTGTGCTGGGCACCCACGATGACTGGCAGATAGGCAAAA
CCAAGATCTTTCTGAAGGACCACCATGACATGCTGCTGGAAGTGGAGCGGGACAAAGCCATC
ACCGACAGAGTCATCCTCCTTCAGAAAGTCATCCGGGGATTCAAAGACAGGTCTAACTTTCT
GAAGCTGAAGAACGCTGCCACACTGATCCAGAGGCACTGGCGGGGTCACAACTGTAGGAAGA
ACTACGGGCTGATGCGTCTGGGCTTCCTGCGGCTGCAGGCCCTGCACCGCTCCCGGAAGCTG
CACCAGCAGTACCGCCTGGCCCGCCAGCGCATCATCCAGTTCCAGGCCCGCTGCCGCGCCTA
TCTGGTGCGCAAGGCCTTCCGCCACCGCCTCTGGGCTGTGCTCACCGTGCAGGCCTATGCCC
GGGGCATGATCGCCCGCAGGCTGCACCAACGCCTCAGGGCTGAGTATCTGTGGCGCCTCGAG
GCTGAGAAAATGCGGCTGGCGGAGGAAGAGAAGCTTCGGAAGGAGATGAGCGCCAAGAAGGC
CAAGGAGGAGGCCGAGCGCAAGCATCAGGAGCGCCTGGCCCAGCTGGCTCGTGAGGACGCTG
AGCGGGAGCTGAAGGAGAAGGAGGCCGCTCGGCGGAAGAAGGAGCTCCTGGAGCAGATGGAA
AGGGCCCGCCATGAGCCTGTCAATCACTCAGACATGGTGGACAAGATGTTTGGCTTCCTGGG
GACTTCAGGTGGCCTGCCAGGCCAGGAGGGCCAGGCACCTAGTGGCTTTGAGGACCTGGAGC
GAGGGCGGAGGGAGATGGTGGAGGAGGACCTGGATGCAGCCCTGCCCCTGCCTGACGAGGAT
GAGGAGGACCTCTCTGAGTATAAATTTGCCAAGTTCGCGGCCACCTACTTCCAGGGGACAAC
TACGCACTCCTACACCCGGCGGCCACTCAAACAGCCACTGCTCTACCATGACGACGAGGGTG
ACCAGCTGGCAGCCCTGGCGGTCTGGATCACCATCCTCCGCTTCATGGGGGACCTCCCTGAG
CCCAAGTACCACACAGCCATGAGTGATGGCAGTGAGAAGATCCCTGTGATGACCAAGATTTA
TGAGACCCTGGGCAAGAAGACGTACAAGAGGGAGCTGCAGGCCCTGCAGGGCGAGGGCGAGG
CCCAGCTCCCCGAGGGCCAGAAGAAGAGCAGTGTGAGGCACAAGCTGGTGCATTTGACTCTG
AAAAAGAAGTCCAAGCTCACAGAGGAGGTGACCAAGAGGCTGCATGACGGGGAGTCCACAGT
GCAGGGCAACAGCATGCTGGAGGACCGGCCCACCTCCAACCTGGAGAAGCTGCACTTCATCA
TCGGCAATGGCATCCTGCGGCCAGCACTCCGGGACGAGATCTACTGCCAGATCAGCAAGCAG
CTGACCCACAACCCCTCCAAGAGCAGCTATGCCCGGGGCTGGATTCTCGTGTCTCTCTGCGT
GGGCTGTTTCGCCCCCTCCGAGAAGTTTGTCAAGTACCTGCGGAACTTCATCCACGGGGGCC
CGCCCGGCTACGCCCCGTACTGTGAGGAGCGCCTGAGAAGGACCTTTGTCAATGGGACACGG
ACACAGCCGCCCAGCTGGCTGGAGCTGCAGGCCACCAAGTCCAAGAAGCCAATCATGTTGCC
CGTGACATTCATGGATGGGACCACCAAGACCCTGCTGACGGACTCGGCAACCACGGCCAAGG
AGCTCTGCAACGCGCTGGCCGACAAGATCTCTCTCAAGGACCGGTTCGGGTTCTCCCTCTAC
ATTGCCCTGTTTGACAAGGTGTCCTCCCTGGGCAGCGGCAGTGACCACGTCATGGACGCCAT
CTCCCAGTGCGAGCAGTACGCCAAGGAGCAGGGCGCCCAGGAGCGCAACGCCCCCTGGAGGC
TCTTCTTCCGCAAAGAGGTCTTCACGCCCTGGCACAGCCCCTCCGAGGACAACGTGGCCACC
AACCTCATCTACCAGCAGGTGGTGCGAGGAGTCAAGTTTGGGGAGTACAGGTGTGAGAAGGA
GGACGACCTGGCTGAGCTGGCCTCCCAGCAGTACTTTGTAGACTATGGCTCTGAGATGATCC
TGGAGCGCCTCCTGAACCTCGTGCCCACCTACATCCCCGACCGCGAGATCACGCCCCTGAAG
ACGCTGGAGAAGTGGGCCCAGCTGGCCATCGCCGCCCACAAGAAGGGGATTTATGCCCAGAG
GAGAACTGATGCCCAGAAGGTCAAAGAGGATGTGGTCAGTTATGCCCGCTTCAAGTGGCCCT
TGCTCTTCTCCAGGTTTTATGAAGCCTACAAATTCTCAGGCCCCAGTCTCCCCAAGAACGAC
GTCATCGTGGCCGTCAACTGGACGGGTGTGTACTTTGTGGATGAGCAGGAGCAGGTACTTCT
GGAGCTGTCCTTCCCAGAGATCATGGCCGTGTCCAGCAGCAGGGAGTGCCGTGTCTGGCTCT
CACTGGGCTGCTCTGATCTTGGCTGTGCTGCGCCTCACTCAGGCTGGGCAGGACTGACCCCG
GCGGGGCCCTGTTCTCCGTGTTGGTCCTGCAGGGGAGCGAAAACGACGGCCCCCAGCTTCAC
GCTGGCCACCATCAAGGGGGACGAATACACCTTCACCTCCAGTAATGCTGAGGACATTCGTG
ACCTGGTGGTCACCTTCCTAGAGGGGCTCCGGAAGAGATCTAAGTATGTTGTGGCCCTGCAG
GATAACCCCAACCCCGCAGGCGAGGAGTCAGGCTTCCTCAGCTTTGCCAAGGGAGACCTCAT
CATCCTGGACCATGACACGGGCGAGCAGGTCATGAACTCGGGCTGGGCCAACGGCATCAATG
AGAGGACCAAGCAGCGTGGGGACTTCCCCACCGACTGTGTGTACGTCATGCCCACTGTCACC
ATGCCACCTCGTGAGATTGTGGCCCTGGTCACCATGACTCCCGATCAGAGGCAGGACGTTGT
CCGGCTCTTGCAGCTGCGAACGGCGGAGCCCGAGGTGCGTGCCAAGCCCTACACGCTGGAGG
AGTTTTCCTATGACTACTTCAGGCCCCCACCCAAGCACACGCTGAGCCGTGTCATGGTGTCC
AAGGCCCGAGGCAAGGACCGGCTGTGGAGCCACACGCGGGAACCGCTCAAGCAGGCGCTGCT
CAAGAAGCTCCTGGGCAGTGAGGAGCTCTCGCAGGAGGCCTGCCTGGCCTTCATTGCTGTGC
TCAAGTACATGGGCGACTACCCGTCCAAGAGGACACGCTCCGTCAATGAGCTCACCGACCAG
ATCTTTGAGGGTCCCCTGAAAGCCGAGCCCCTGAAGGACGAGGCATATGTGCAGATCCTGAA
GCAGCTGACCGACAACCACATCAGGTACAGCGAGGAGCGGGGTTGGGAGCTGCTCTGGCTGT
GCACGGGCCTTTTCCCACCCAGCAACATCCTCCTGCCCCACGTGCAGCGCTTCCTGCAGTCC
CGAAAGCACTGCCCACTCGCCATCGACTGCCTGCAACGGCTCCAGAAAGCCCTGAGAAACGG
GTCCCGGAAGTACCCTCCGCACCTGGTGGAGGTGGAGGCCATCCAGCACAAGACCACCCAGA
TTTTCCACAAGGTCTACTTCCCTGATGACACTGACGAGGCCTTCGAAGTGGAGTCCAGCACC
AAGGCCAAGGACTTCTGCCAGAACATCGCCACCAGGCTGCTCCTCAAGTCCTCAGAGGGATT
CAGCCTCTTTGTCAAAATTGCAGACAAGGTCATCAGCGTTCCTGAGAATGACTTCTTCTTTG
ACTTTGTTCGACACTTGACAGACTGGATAAAGAAAGCTCGGCCCATCAAGGACGGAATTGTG
CCCTCACTCACCTACCAGGTGTTCTTCATGAAGAAGCTGTGGACCACCACGGTGCCAGGGAA
GGATCCCATGGCCGATTCCATCTTCCACTATTACCAGGAGTTGCCCAAGTATCTCCGAGGCT
ACCACAAGTGCACGCGGGAGGAGGTGCTGCAGCTGGGGGCGCTGATCTACAGGGTCAAGTTC
GAGGAGGACAAGTCCTACTTCCCCAGCATCCCCAAGCTGCTGCGGGAGCTGGTGCCCCAGGA
CCTTATCCGGCAGGTCTCACCTGATGACTGGAAGCGGTCCATCGTCGCCTACTTCAACAAGC
ACGCAGGGAAGTCCAAGGAGGAGGCCAAGCTGGCCTTCCTGAAGCTCATCTTCAAGTGGCCC
ACCTTTGGCTCAGCCTTCTTCGAGGTGAAGCAAACTACGGAGCCAAACTTCCCTGAGATCCT
CCTAATTGCCATCAACAAGTATGGGGTCAGCCTCATCGATCCCAAAACGAAGGATATCCTCA
CCACTCATCCCTTCACCAAGATCTCCAACTGGAGCAGCGGCAACACCTACTTCCACATCACC
ATTGGGAACTTGGTGCGCGGGAGCAAACTGCTCTGCGAGACGTCACTGGGCTACAAGATGGA
TGACCTCCTGACTTCCTACATTAGCCAGATGCTCACAGCCATGAGCAAACAGCGGGGCTCCA
GGAGCGGCAAGTGA

An example 5′ end portion of a MYO7A transgene is:

(SEQ ID NO: 12)
ATGGTGATTCTTCAGCAGGGGGACCATGTGTGGATGGACCTGAGATTGGGGCAGGAGTTCGA
CGTGCCCATCGGGGCGGTGGTGAAGCTCTGCGACTCTGGGCAGGTCCAGGTGGTGGATGATG
AAGACAATGAACACTGGATCTCTCCGCAGAACGCAACGCACATCAAGCCTATGCACCCCACG
TCGGTCCACGGCGTGGAGGACATGATCCGCCTGGGGGACCTCAACGAGGCGGGCATCTTGCG
CAACCTGCTTATCCGCTACCGGGACCACCTCATCTACACGTATACGGGCTCCATCCTGGTGG
CTGTGAACCCCTACCAGCTGCTCTCCATCTACTCGCCAGAGCACATCCGCCAGTATACCAAC
AAGAAGATTGGGGAGATGCCCCCCCACATCTTTGCCATTGCTGACAACTGCTACTTCAACAT
GAAACGCAACAGCCGAGACCAGTGCTGCATCATCAGTGGGGAATCTGGGGCCGGGAAGACGG
AGAGCACAAAGCTGATCCTGCAGTTCCTGGCAGCCATCAGTGGGCAGCACTCGTGGATTGAG
CAGCAGGTCTTGGAGGCCACCCCCATTCTGGAAGCATTTGGGAATGCCAAGACCATCCGCAA
TGACAACTCAAGCCGTTTCGGAAAGTACATCGACATCCACTTCAACAAGCGGGGCGCCATCG
AGGGCGCGAAGATTGAGCAGTACCTGCTGGAAAAGTCACGTGTCTGTCGCCAGGCCCTGGAT
GAAAGGAACTACCACGTGTTCTACTGCATGCTGGAGGGCATGAGTGAGGATCAGAAGAAGAA
GCTGGGCTTGGGCCAGGCCTCTGACTACAACTACTTGGCCATGGGTAACTGCATAACCTGTG
AGGGCCGGGTGGACAGCCAGGAGTACGCCAACATCCGCTCCGCCATGAAGGTGCTCATGTTC
ACTGACACCGAGAACTGGGAGATCTCGAAGCTCCTGGCTGCCATCCTGCACCTGGGCAACCT
GCAGTATGAGGCACGCACATTTGAAAACCTGGATGCCTGTGAGGTTCTCTTCTCCCCATCGC
TGGCCACAGCTGCATCCCTGCTTGAGGTGAACCCCCCAGACCTGATGAGCTGCCTGACTAGC
CGCACCCTCATCACCCGCGGGGAGACGGTGTCCACCCCACTGAGCAGGGAACAGGCACTGGA
CGTGCGCGACGCCTTCGTAAAGGGGATCTACGGGCGGCTGTTCGTGTGGATTGTGGACAAGA
TCAACGCAGCAATTTACAAGCCTCCCTCCCAGGATGTGAAGAACTCTCGCAGGTCCATCGGC
CTCCTGGACATCTTTGGGTTTGAGAACTTTGCTGTGAACAGCTTTGAGCAGCTCTGCATCAA
CTTCGCCAATGAGCACCTGCAGCAGTTCTTTGTGCGGCACGTGTTCAAGCTGGAGCAGGAGG
AATATGACCTGGAGAGCATTGACTGGCTGCACATCGAGTTCACTGACAACCAGGATGCCCTG
GAGATGATTGCCAACAAGCCCATGAACATCATCTCCCTCATCGATGAGGAGAGCAAGTTCCC
CAAGGGCACAGACACCACCATGTTACACAAGCTGAACTCCCAGCACAAGCTCAACGCCAACT
ACATCCCCCCCAAGAACAACCATGAGACCCAGTTTGGCATCAACCATTTTGCAGGCATCGTC
TACTATGAGACCCAAGGCTTCCTGGAGAAGAACCGAGACACCCTGCATGGGGACATTATCCA
GCTGGTCCACTCCTCCAGGAACAAGTTCATCAAGCAGATCTTCCAGGCCGATGTCGCCATGG
GCGCCGAGACCAGGAAGCGCTCGCCCACACTTAGCAGCCAGTTCAAGCGGTCACTGGAGCTG
CTGATGCGCACGCTGGGTGCCTGCCAGCCCTTCTTTGTGCGATGCATCAAGCCCAATGAGTT
CAAGAAGCCCATGCTGTTCGACCGGCACCTGTGCGTGCGCCAGCTGCGGTACTCAGGAATGA
TGGAGACCATCCGAATCCGCCGAGCTGGCTACCCCATCCGCTACAGCTTCGTAGAGTTTGTG
GAGCGGTACCGTGTGCTGCTGCCAGGTGTGAAGCCGGCCTACAAGCAGGGCGACCTCCGCGG
GACTTGCCAGCGCATGGCTGAGGCTGTGCTGGGCACCCACGATGACTGGCAGATAGGCAAAA
CCAAGATCTTTCTGAAGGACCACCATGACATGCTGCTGGAAGTGGAGCGGGACAAAGCCATC
ACCGACAGAGTCATCCTCCTTCAGAAAGTCATCCGGGGATTCAAAGACAGGTCTAACTTTCT
GAAGCTGAAGAACGCTGCCACACTGATCCAGAGGCACTGGCGGGGTCACAACTGTAGGAAGA
ACTACGGGCTGATGCGTCTGGGCTTCCTGCGGCTGCAGGCCCTGCACCGCTCCCGGAAGCTG
CACCAGCAGTACCGCCTGGCCCGCCAGCGCATCATCCAGTTCCAGGCCCGCTGCCGCGCCTA
TCTGGTGCGCAAGGCCTTCCGCCACCGCCTCTGGGCTGTGCTCACCGTGCAGGCCTATGCCC
GGGGCATGATCGCCCGCAGGCTGCACCAACGCCTCAGGGCTGAGTATCTGTGGCGCCTCGAG
GCTGAGAAAATGCGGCTGGCGGAGGAAGAGAAGCTTCGGAAGGAGATGAGCGCCAAGAAGGC
CAAGGAGGAGGCCGAGCGCAAGCATCAGGAGCGCCTGGCCCAGCTGGCTCGTGAGGACGCTG
AGCGGGAGCTGAAGGAGAAGGAGGCCGCTCGGCGGAAGAAGGAGCTCCTGGAGCAGATGGAA
AGGGCCCGCCATGAGCCTGTCAATCACTCAGACATGGTGGACAAGATGTTTGGCTTCCTGGG
GACTTCAGGTGGCCTGCCAGGCCAGGAGGGCCAGGCACCTAGTGGCTTTGAGGACCTGGAGC
GAGGGCGGAGGGAGATGGTGGAGGAGGACCTGGATGCAGCCCTGCCCCTGCCTGACGAGGAT
GAGGAGGACCTCTCTGAGTATAAATTTGCCAAGTTCGCGGCCACCTACTTCCAGGGGACAAC
TACGCACTCCTACACCCGGCGGCCACTCAAACAGCCACTGCTCTACCATGACGACGAGGGTG
ACCAGCTG

In some embodiments, the 5′ end portion of the transgene comprises or consists of a nucleic acid sequence that has at least 70%, 80%, 90%, 95%, 96%, 97%, 98%, 99% or 100% nucleotide identity to SEQ ID NO: 12, preferably wherein the 5′ end portion of the transgene substantially retains the natural function of the 5′ end portion of the transgene of SEQ ID NO: 12.

In preferred embodiments, the 5′ end portion of the transgene comprises or consists of the nucleic acid sequence of SEQ ID NO: 12.

In preferred embodiments, the first vector comprises a 5′ end portion of the transgene that comprises or consists of the nucleic acid sequence of SEQ ID NO: 12.

An example 3′ end portion of a MYO7A transgene is:

(SEQ ID NO: 13)
GCAGCCCTGGCGGTCTGGATCACCATCCTCCGCTTCATGGGGGACCTCCCTGAGCCCAAGTA
CCACACAGCCATGAGTGATGGCAGTGAGAAGATCCCTGTGATGACCAAGATTTATGAGACCC
TGGGCAAGAAGACGTACAAGAGGGAGCTGCAGGCCCTGCAGGGCGAGGGCGAGGCCCAGCTC
CCCGAGGGCCAGAAGAAGAGCAGTGTGAGGCACAAGCTGGTGCATTTGACTCTGAAAAAGAA
GTCCAAGCTCACAGAGGAGGTGACCAAGAGGCTGCATGACGGGGAGTCCACAGTGCAGGGCA
ACAGCATGCTGGAGGACCGGCCCACCTCCAACCTGGAGAAGCTGCACTTCATCATCGGCAAT
GGCATCCTGCGGCCAGCACTCCGGGACGAGATCTACTGCCAGATCAGCAAGCAGCTGACCCA
CAACCCCTCCAAGAGCAGCTATGCCCGGGGCTGGATTCTCGTGTCTCTCTGCGTGGGCTGTT
TCGCCCCCTCCGAGAAGTTTGTCAAGTACCTGCGGAACTTCATCCACGGGGGCCCGCCCGGC
TACGCCCCGTACTGTGAGGAGCGCCTGAGAAGGACCTTTGTCAATGGGACACGGACACAGCC
GCCCAGCTGGCTGGAGCTGCAGGCCACCAAGTCCAAGAAGCCAATCATGTTGCCCGTGACAT
TCATGGATGGGACCACCAAGACCCTGCTGACGGACTCGGCAACCACGGCCAAGGAGCTCTGC
AACGCGCTGGCCGACAAGATCTCTCTCAAGGACCGGTTCGGGTTCTCCCTCTACATTGCCCT
GTTTGACAAGGTGTCCTCCCTGGGCAGCGGCAGTGACCACGTCATGGACGCCATCTCCCAGT
GCGAGCAGTACGCCAAGGAGCAGGGCGCCCAGGAGCGCAACGCCCCCTGGAGGCTCTTCTTC
CGCAAAGAGGTCTTCACGCCCTGGCACAGCCCCTCCGAGGACAACGTGGCCACCAACCTCAT
CTACCAGCAGGTGGTGCGAGGAGTCAAGTTTGGGGAGTACAGGTGTGAGAAGGAGGACGACC
TGGCTGAGCTGGCCTCCCAGCAGTACTTTGTAGACTATGGCTCTGAGATGATCCTGGAGCGC
CTCCTGAACCTCGTGCCCACCTACATCCCCGACCGCGAGATCACGCCCCTGAAGACGCTGGA
GAAGTGGGCCCAGCTGGCCATCGCCGCCCACAAGAAGGGGATTTATGCCCAGAGGAGAACTG
ATGCCCAGAAGGTCAAAGAGGATGTGGTCAGTTATGCCCGCTTCAAGTGGCCCTTGCTCTTC
TCCAGGTTTTATGAAGCCTACAAATTCTCAGGCCCCAGTCTCCCCAAGAACGACGTCATCGT
GGCCGTCAACTGGACGGGTGTGTACTTTGTGGATGAGCAGGAGCAGGTACTTCTGGAGCTGT
CCTTCCCAGAGATCATGGCCGTGTCCAGCAGCAGGGAGTGCCGTGTCTGGCTCTCACTGGGC
TGCTCTGATCTTGGCTGTGCTGCGCCTCACTCAGGCTGGGCAGGACTGACCCCGGCGGGGCC
CTGTTCTCCGTGTTGGTCCTGCAGGGGAGCGAAAACGACGGCCCCCAGCTTCACGCTGGCCA
CCATCAAGGGGGACGAATACACCTTCACCTCCAGTAATGCTGAGGACATTCGTGACCTGGTG
GTCACCTTCCTAGAGGGGCTCCGGAAGAGATCTAAGTATGTTGTGGCCCTGCAGGATAACCC
CAACCCCGCAGGCGAGGAGTCAGGCTTCCTCAGCTTTGCCAAGGGAGACCTCATCATCCTGG
ACCATGACACGGGCGAGCAGGTCATGAACTCGGGCTGGGCCAACGGCATCAATGAGAGGACC
AAGCAGCGTGGGGACTTCCCCACCGACTGTGTGTACGTCATGCCCACTGTCACCATGCCACC
TCGTGAGATTGTGGCCCTGGTCACCATGACTCCCGATCAGAGGCAGGACGTTGTCCGGCTCT
TGCAGCTGCGAACGGCGGAGCCCGAGGTGCGTGCCAAGCCCTACACGCTGGAGGAGTTTTCC
TATGACTACTTCAGGCCCCCACCCAAGCACACGCTGAGCCGTGTCATGGTGTCCAAGGCCCG
AGGCAAGGACCGGCTGTGGAGCCACACGCGGGAACCGCTCAAGCAGGCGCTGCTCAAGAAGC
TCCTGGGCAGTGAGGAGCTCTCGCAGGAGGCCTGCCTGGCCTTCATTGCTGTGCTCAAGTAC
ATGGGCGACTACCCGTCCAAGAGGACACGCTCCGTCAATGAGCTCACCGACCAGATCTTTGA
GGGTCCCCTGAAAGCCGAGCCCCTGAAGGACGAGGCATATGTGCAGATCCTGAAGCAGCTGA
CCGACAACCACATCAGGTACAGCGAGGAGCGGGGTTGGGAGCTGCTCTGGCTGTGCACGGGC
CTTTTCCCACCCAGCAACATCCTCCTGCCCCACGTGCAGCGCTTCCTGCAGTCCCGAAAGCA
CTGCCCACTCGCCATCGACTGCCTGCAACGGCTCCAGAAAGCCCTGAGAAACGGGTCCCGGA
AGTACCCTCCGCACCTGGTGGAGGTGGAGGCCATCCAGCACAAGACCACCCAGATTTTCCAC
AAGGTCTACTTCCCTGATGACACTGACGAGGCCTTCGAAGTGGAGTCCAGCACCAAGGCCAA
GGACTTCTGCCAGAACATCGCCACCAGGCTGCTCCTCAAGTCCTCAGAGGGATTCAGCCTCT
TTGTCAAAATTGCAGACAAGGTCATCAGCGTTCCTGAGAATGACTTCTTCTTTGACTTTGTT
CGACACTTGACAGACTGGATAAAGAAAGCTCGGCCCATCAAGGACGGAATTGTGCCCTCACT
CACCTACCAGGTGTTCTTCATGAAGAAGCTGTGGACCACCACGGTGCCAGGGAAGGATCCCA
TGGCCGATTCCATCTTCCACTATTACCAGGAGTTGCCCAAGTATCTCCGAGGCTACCACAAG
TGCACGCGGGAGGAGGTGCTGCAGCTGGGGGCGCTGATCTACAGGGTCAAGTTCGAGGAGGA
CAAGTCCTACTTCCCCAGCATCCCCAAGCTGCTGCGGGAGCTGGTGCCCCAGGACCTTATCC
GGCAGGTCTCACCTGATGACTGGAAGCGGTCCATCGTCGCCTACTTCAACAAGCACGCAGGG
AAGTCCAAGGAGGAGGCCAAGCTGGCCTTCCTGAAGCTCATCTTCAAGTGGCCCACCTTTGG
CTCAGCCTTCTTCGAGGTGAAGCAAACTACGGAGCCAAACTTCCCTGAGATCCTCCTAATTG
CCATCAACAAGTATGGGGTCAGCCTCATCGATCCCAAAACGAAGGATATCCTCACCACTCAT
CCCTTCACCAAGATCTCCAACTGGAGCAGCGGCAACACCTACTTCCACATCACCATTGGGAA
CTTGGTGCGCGGGAGCAAACTGCTCTGCGAGACGTCACTGGGCTACAAGATGGATGACCTCC
TGACTTCCTACATTAGCCAGATGCTCACAGCCATGAGCAAACAGCGGGGCTCCAGGAGCGGC
AAGTGA

In some embodiments, the 3′ end portion of the transgene comprises or consists of a nucleic acid sequence that has at least 70%, 80%, 90%, 95%, 96%, 97%, 98%, 99% or 100% nucleotide identity to SEQ ID NO: 13, preferably wherein the 3′ end portion of the transgene substantially retains the natural function of the 3′ end portion of the transgene of SEQ ID NO: 13.

In preferred embodiments, the 3′ end portion of the transgene comprises or consists of the nucleic acid sequence of SEQ ID NO: 13.

In preferred embodiments, the first vector comprises a 3′ end portion of the transgene that comprises or consists of the nucleic acid sequence of SEQ ID NO: 13.

A further example MYO7A nucleotide sequence is:

ATGGTGATTCTTCAGCAGGGGGACCATGTGTGGATGGACCTGAGATTGGGGCAGGAGTTCGA
CGTGCCCATCGGGGCGGTGGTGAAGCTCTGCGACTCTGGGCAGGTCCAGGTGGTGGATGATG
AAGACAATGAACACTGGATCTCTCCGCAGAACGCAACGCACATCAAGCCTATGCACCCCACG
TCGGTCCACGGCGTGGAGGACATGATCCGCCTGGGGGACCTCAACGAGGCGGGCATCTTGCG
CAACCTGCTTATCCGCTACCGGGACCACCTCATCTACACGTATACGGGCTCCATCCTGGTGG
CTGTGAACCCCTACCAGCTGCTCTCCATCTACTCGCCAGAGCACATCCGCCAGTATACCAAC
AAGAAGATTGGGGAGATGCCCCCCCACATCTTTGCCATTGCTGACAACTGCTACTTCAACAT
GAAACGCAACAGCCGAGACCAGTGCTGCATCATCAGTGGGGAATCTGGGGCCGGGAAGACGG
AGAGCACAAAGCTGATCCTGCAGTTCCTGGCAGCCATCAGTGGGCAGCACTCGTGGATTGAG
CAGCAGGTCTTGGAGGCCACCCCCATTCTGGAAGCATTTGGGAATGCCAAGACCATCCGCAA
TGACAACTCAAGCCGTTTCGGAAAGTACATCGACATCCACTTCAACAAGCGGGGCGCCATCG
AGGGCGCGAAGATTGAGCAGTACCTGCTGGAAAAGTCACGTGTCTGTCGCCAGGCCCTGGAT
GAAAGGAACTACCACGTGTTCTACTGCATGCTGGAGGGTATGAGTGAGGATCAGAAGAAGAA
GCTGGGCTTGGGCCAGGCCTCTGACTACAACTACTTGGCCATGGGTAACTGCATAACCTGTG
AGGGCCGGGTGGACAGCCAGGAGTACGCCAACATCCGCTCCGCCATGAAGGTGCTCATGTTC
ACTGACACCGAGAACTGGGAGATCTCGAAGCTCCTGGCTGCCATCCTGCACCTGGGCAACCT
GCAGTATGAGGCACGCACATTTGAAAACCTGGATGCCTGTGAGGTTCTCTTCTCCCCATCGC
TGGCCACAGCTGCATCCCTGCTTGAGGTGAACCCCCCAGACCTGATGAGCTGCCTGACTAGC
CGCACCCTCATCACCCGCGGGGAGACGGTGTCCACCCCACTGAGCAGGGAACAGGCACTGGA
CGTGCGCGACGCCTTCGTAAAGGGGATCTACGGGCGGCTGTTCGTGTGGATTGTGGACAAGA
TCAACGCAGCAATTTACAAGCCTCCCTCCCAGGATGTGAAGAACTCTCGCAGGTCCATCGGC
CTCCTGGACATCTTTGGGTTTGAGAACTTTGCTGTGAACAGCTTTGAGCAGCTCTGCATCAA
CTTCGCCAATGAGCACCTGCAGCAGTTCTTTGTGCGGCACGTGTTCAAGCTGGAGCAGGAGG
AATATGACCTGGAGAGCATTGACTGGCTGCACATCGAGTTCACTGACAACCAGGATGCCCTG
GAGATGATTGCCAACAAGCCCATGAACATCATCTCCCTCATCGATGAGGAGAGCAAGTTCCC
CAAGGGCACAGACACCACCATGTTACACAAGCTGAACTCCCAGCACAAGCTCAACGCCAACT
ACATCCCCCCCAAGAACAACCATGAGACCCAGTTTGGCATCAACCATTTTGCAGGCATCGTC
TACTATGAGACCCAAGGCTTCCTGGAGAAGAACCGAGACACCCTGCATGGGGACATTATCCA
GCTGGTCCACTCCTCCAGGAACAAGTTCATCAAGCAGATCTTCCAGGCCGATGTCGCCATGG
GCGCCGAGACCAGGAAGCGCTCGCCCACACTTAGCAGCCAGTTCAAGCGGTCACTGGAGCTG
CTGATGCGCACGCTGGGTGCCTGCCAGCCCTTCTTTGTGCGATGCATCAAGCCCAATGAGTT
CAAGAAGCCCATGCTGTTCGACCGGCACCTGTGCGTGCGCCAGCTGCGGTACTCAGGAATGA
TGGAGACCATCCGAATCCGCCGAGCTGGCTACCCCATCCGCTACAGCTTCGTAGAGTTTGTG
GAGCGGTACCGTGTGCTGCTGCCAGGTGTGAAGCCGGCCTACAAGCAGGGCGACCTCCGCGG
GACTTGCCAGCGCATGGCTGAGGCTGTGCTGGGCACCCACGATGACTGGCAGATAGGCAAAA
CCAAGATCTTTCTGAAGGACCACCATGACATGCTGCTGGAAGTGGAGCGGGACAAAGCCATC
ACCGACAGAGTCATCCTCCTTCAGAAAGTCATCCGGGGATTCAAAGACAGGTCTAACTTTCT
GAAGCTGAAGAACGCTGCCACACTGATCCAGAGGCACTGGCGGGGTCACAACTGTAGGAAGA
ACTACGGGCTGATGCGTCTGGGCTTCCTGCGGCTGCAGGCCCTGCACCGCTCCCGGAAGCTG
CACCAGCAGTACCGCCTGGCCCGCCAGCGCATCATCCAGTTCCAGGCCCGCTGCCGCGCCTA
TCTGGTGCGCAAGGCCTTCCGCCACCGCCTCTGGGCTGTGCTCACCGTGCAGGCCTATGCCC
GGGGCATGATCGCCCGCAGGCTGCACCAACGCCTCAGGGCTGAGTATCTGTGGCGCCTCGAG
GCTGAGAAAATGCGGCTGGCGGAGGAAGAGAAGCTTCGGAAGGAGATGAGCGCCAAGAAGGC
CAAGGAGGAGGCCGAGCGCAAGCATCAGGAGCGCCTGGCCCAGCTGGCTCGTGAGGACGCTG
AGCGGGAGCTGAAGGAGAAGGAGGCCGCTCGGCGGAAGAAGGAGCTCCTGGAGCAGATGGAA
AGGGCCCGCCATGAGCCTGTCAATCACTCAGACATGGTGGACAAGATGTTTGGCTTCCTGGG
GACTTCAGGTGGCCTGCCAGGCCAGGAGGGCCAGGCACCTAGTGGCTTTGAGGACCTGGAGC
GAGGGCGGAGGGAGATGGTGGAGGAGGACCTGGATGCAGCCCTGCCCCTGCCTGACGAGGAT
GAGGAGGACCTCTCTGAGTATAAATTTGCCAAGTTCGCGGCCACCTACTTCCAGGGGACAAC
CACGCACTCCTACACCCGGCGGCCACTCAAACAGCCACTGCTCTACCATGACGACGAGGGTG
ACCAGCTGGCAGCCCTGGCGGTCTGGATCACCATCCTCCGCTTCATGGGGGACCTCCCTGAG
CCCAAGTACCACACAGCCATGAGTGATGGCAGTGAGAAGATCCCTGTGATGACCAAGATTTA
TGAGACCCTGGGCAAGAAGACGTACAAGAGGGAGCTGCAGGCCCTGCAGGGCGAGGGCGAGG
CCCAGCTCCCCGAGGGCCAGAAGAAGAGCAGTGTGAGGCACAAGCTGGTGCATTTGACTCTG
AAAAAGAAGTCCAAGCTCACAGAGGAGGTGACCAAGAGGCTGCATGACGGGGAGTCCACAGT
GCAGGGCAACAGCATGCTGGAGGACCGGCCCACCTCCAACCTGGAGAAGCTGCACTTCATCA
TCGGCAATGGCATCCTGCGGCCAGCACTCCGGGACGAGATCTACTGCCAGATCAGCAAGCAG
CTGACCCACAACCCCTCCAAGAGCAGCTATGCCCGGGGCTGGATTCTCGTGTCTCTCTGCGT
GGGCTGTTTCGCCCCCTCCGAGAAGTTTGTCAAGTACCTGCGGAACTTCATCCACGGGGGCC
CGCCCGGCTACGCCCCGTACTGTGAGGAGCGCCTGAGAAGGACCTTTGTCAATGGGACACGG
ACACAGCCGCCCAGCTGGCTGGAGCTGCAGGCCACCAAGTCCAAGAAGCCAATCATGTTGCC
CGTGACATTCATGGATGGGACCACCAAGACCCTGCTGACGGACTCGGCAACCACGGCCAAGG
AGCTCTGCAACGCGCTGGCCGACAAGATCTCTCTCAAGGACCGGTTCGGGTTCTCCCTCTAC
ATTGCCCTGTTTGACAAGGTGTCCTCCCTGGGCAGCGGCAGTGACCACGTCATGGACGCCAT
CTCCCAGTGCGAGCAGTACGCCAAGGAGCAGGGCGCCCAGGAGCGCAACGCCCCCTGGAGGC
TCTTCTTCCGCAAAGAGGTCTTCACGCCCTGGCACAGCCCCTCCGAGGACAACGTGGCCACC
AACCTCATCTACCAGCAGGTGGTGCGAGGAGTCAAGTTTGGGGAGTACAGGTGTGAGAAGGA
GGACGACCTGGCTGAGCTGGCCTCCCAGCAGTACTTTGTAGACTATGGCTCTGAGATGATCC
TGGAGCGCCTCCTGAACCTCGTGCCCACCTACATCCCCGACCGCGAGATCACGCCCCTGAAG
ACGCTGGAGAAGTGGGCCCAGCTGGCCATCGCCGCCCACAAGAAGGGGATTTATGCCCAGAG
GAGAACTGATGCCCAGAAGGTCAAAGAGGATGTGGTCAGTTATGCCCGCTTCAAGTGGCCCT
TGCTCTTCTCCAGGTTTTATGAAGCCTACAAATTCTCAGGCCCCAGTCTCCCCAAGAACGAC
GTCATCGTGGCCGTCAACTGGACGGGTGTGTACTTTGTGGATGAGCAGGAGCAGGTACTTCT
GGAGCTGTCCTTCCCAGAGATCATGGCCGTGTCCAGCAGCAGGGAGTGCCGTGTCTGGCTCT
CACTGGGCTGCTCTGATCTTGGCTGTGCTGCGCCTCACTCAGGCTGGGCAGGACTGACCCCG
GCGGGGCCCTGTTCTCCGTGTTGGTCCTGCAGGGGAGCGAAAACGACGGCCCCCAGCTTCAC
GCTGGCCACCATCAAGGGGGACGAATACACCTTCACCTCCAGCAATGCTGAGGACATTCGTG
ACCTGGTGGTCACCTTCCTAGAGGGGCTCCGGAAGAGATCTAAGTATGTTGTGGCCCTGCAG
GATAACCCCAACCCCGCAGGCGAGGAGTCAGGCTTCCTCAGCTTTGCCAAGGGAGACCTCAT
CATCCTGGACCATGACACGGGCGAGCAGGTCATGAACTCGGGCTGGGCCAACGGCATCAATG
AGAGGACCAAGCAGCGTGGGGACTTCCCCACCGACAGTGTGTACGTCATGCCCACTGTCACC
ATGCCACCGCGGGAGATTGTGGCCCTGGTCACCATGACTCCCGATCAGAGGCAGGACGTTGT
CCGGCTCTTGCAGCTGCGAACGGCGGAGCCCGAGGTGCGTGCCAAGCCCTACACGCTGGAGG
AGTTTTCCTATGACTACTTCAGGCCCCCACCCAAGCACACGCTGAGCCGTGTCATGGTGTCC
AAGGCCCGAGGCAAGGACCGGCTGTGGAGCCACACGCGGGAACCGCTCAAGCAGGCGCTGCT
CAAGAAGCTCCTGGGCAGTGAGGAGCTCTCGCAGGAGGCCTGCCTGGCCTTCATTGCTGTGC
TCAAGTACATGGGCGACTACCCGTCCAAGAGGACACGCTCCGTCAACGAGCTCACCGACCAG
ATCTTTGAGGGTCCCCTGAAAGCCGAGCCCCTGAAGGACGAGGCATATGTGCAGATCCTGAA
GCAGCTGACCGACAACCACATCAGGTACAGCGAGGAGCGGGGTTGGGAGCTGCTCTGGCTGT
GCACGGGCCTTTTCCCACCCAGCAACATCCTCCTGCCCCACGTGCAGCGCTTCCTGCAGTCC
CGAAAGCACTGCCCACTCGCCATCGACTGCCTGCAACGGCTCCAGAAAGCCCTGAGAAACGG
GTCCCGGAAGTACCCTCCGCACCTGGTGGAGGTGGAGGCCATCCAGCACAAGACCACCCAGA
TTTTCCACAAAGTCTACTTCCCTGATGACACTGACGAGGCCTTCGAAGTGGAGTCCAGCACC
AAGGCCAAGGACTTCTGCCAGAACATCGCCACCAGGCTGCTCCTCAAGTCCTCAGAGGGATT
CAGCCTCTTTGTCAAAATTGCAGACAAGGTCCTCAGCGTTCCTGAGAATGACTTCTTCTTTG
ACTTTGTTCGACACTTGACAGACTGGATAAAGAAAGCTCGGCCCATCAAGGACGGAATTGTG
CCCTCACTCACCTACCAGGTGTTCTTCATGAAGAAGCTGTGGACCACCACGGTGCCAGGGAA
GGATCCCATGGCCGATTCCATCTTCCACTATTACCAGGAGTTGCCCAAGTATCTCCGAGGCT
ACCACAAGTGCACGCGGGAGGAGGTGCTGCAGCTGGGGGCGCTGATCTACAGGGTCAAGTTC
GAGGAGGACAAGTCCTACTTCCCCAGCATCCCCAAGCTGCTGCGGGAGCTGGTGCCCCAGGA
CCTTATCCGGCAGGTCTCACCTGATGACTGGAAGCGGTCCATCGTCGCCTACTTCAACAAGC
ACGCAGGGAAGTCCAAGGAGGAGGCCAAGCTGGCCTTCCTGAAGCTCATCTTCAAGTGGCCC
ACCTTTGGCTCAGCCTTCTTCGAGGTGAAGCAAACTACGGAGCCAAACTTCCCTGAGATCCT
CCTAATTGCCATCAACAAGTATGGGGTCAGCCTCATCGATCCCAAAACGAAGGATATCCTCA
CCACTCATCCCTTCACCAAGATCTCCAACTGGAGCAGCGGCAACACCTACTTCCACATCACC
ATTGGGAACTTGGTGCGCGGGAGCAAACTGCTCTGCGAGACGTCACTGGGCTACAAGATGGA
TGACCTCCTGACTTCCTACATTAGCCAGATGCTCACAGCCATGAGCAAACAGCGGGGCTCCA
GGAGCGGCAAGTGA
(SEQ ID NO: 39; NM_000260.3 nucleotides 273-6920)

A further example 5′ end portion of a MYO7A transgene is:

ATGGTGATTCTTCAGCAGGGGGACCATGTGTGGATGGACCTGAGATTGGGGCAGGAGTTCGA
CGTGCCCATCGGGGCGGTGGTGAAGCTCTGCGACTCTGGGCAGGTCCAGGTGGTGGATGATG
AAGACAATGAACACTGGATCTCTCCGCAGAACGCAACGCACATCAAGCCTATGCACCCCACG
TCGGTCCACGGCGTGGAGGACATGATCCGCCTGGGGGACCTCAACGAGGCGGGCATCTTGCG
CAACCTGCTTATCCGCTACCGGGACCACCTCATCTACACGTATACGGGCTCCATCCTGGTGG
CTGTGAACCCCTACCAGCTGCTCTCCATCTACTCGCCAGAGCACATCCGCCAGTATACCAAC
AAGAAGATTGGGGAGATGCCCCCCCACATCTTTGCCATTGCTGACAACTGCTACTTCAACAT
GAAACGCAACAGCCGAGACCAGTGCTGCATCATCAGTGGGGAATCTGGGGCCGGGAAGACGG
AGAGCACAAAGCTGATCCTGCAGTTCCTGGCAGCCATCAGTGGGCAGCACTCGTGGATTGAG
CAGCAGGTCTTGGAGGCCACCCCCATTCTGGAAGCATTTGGGAATGCCAAGACCATCCGCAA
TGACAACTCAAGCCGTTTCGGAAAGTACATCGACATCCACTTCAACAAGCGGGGCGCCATCG
AGGGCGCGAAGATTGAGCAGTACCTGCTGGAAAAGTCACGTGTCTGTCGCCAGGCCCTGGAT
GAAAGGAACTACCACGTGTTCTACTGCATGCTGGAGGGTATGAGTGAGGATCAGAAGAAGAA
GCTGGGCTTGGGCCAGGCCTCTGACTACAACTACTTGGCCATGGGTAACTGCATAACCTGTG
AGGGCCGGGTGGACAGCCAGGAGTACGCCAACATCCGCTCCGCCATGAAGGTGCTCATGTTC
ACTGACACCGAGAACTGGGAGATCTCGAAGCTCCTGGCTGCCATCCTGCACCTGGGCAACCT
GCAGTATGAGGCACGCACATTTGAAAACCTGGATGCCTGTGAGGTTCTCTTCTCCCCATCGC
TGGCCACAGCTGCATCCCTGCTTGAGGTGAACCCCCCAGACCTGATGAGCTGCCTGACTAGC
CGCACCCTCATCACCCGCGGGGAGACGGTGTCCACCCCACTGAGCAGGGAACAGGCACTGGA
CGTGCGCGACGCCTTCGTAAAGGGGATCTACGGGCGGCTGTTCGTGTGGATTGTGGACAAGA
TCAACGCAGCAATTTACAAGCCTCCCTCCCAGGATGTGAAGAACTCTCGCAGGTCCATCGGC
CTCCTGGACATCTTTGGGTTTGAGAACTTTGCTGTGAACAGCTTTGAGCAGCTCTGCATCAA
CTTCGCCAATGAGCACCTGCAGCAGTTCTTTGTGCGGCACGTGTTCAAGCTGGAGCAGGAGG
AATATGACCTGGAGAGCATTGACTGGCTGCACATCGAGTTCACTGACAACCAGGATGCCCTG
GAGATGATTGCCAACAAGCCCATGAACATCATCTCCCTCATCGATGAGGAGAGCAAGTTCCC
CAAGGGCACAGACACCACCATGTTACACAAGCTGAACTCCCAGCACAAGCTCAACGCCAACT
ACATCCCCCCCAAGAACAACCATGAGACCCAGTTTGGCATCAACCATTTTGCAGGCATCGTC
TACTATGAGACCCAAGGCTTCCTGGAGAAGAACCGAGACACCCTGCATGGGGACATTATCCA
GCTGGTCCACTCCTCCAGGAACAAGTTCATCAAGCAGATCTTCCAGGCCGATGTCGCCATGG
GCGCCGAGACCAGGAAGCGCTCGCCCACACTTAGCAGCCAGTTCAAGCGGTCACTGGAGCTG
CTGATGCGCACGCTGGGTGCCTGCCAGCCCTTCTTTGTGCGATGCATCAAGCCCAATGAGTT
CAAGAAGCCCATGCTGTTCGACCGGCACCTGTGCGTGCGCCAGCTGCGGTACTCAGGAATGA
TGGAGACCATCCGAATCCGCCGAGCTGGCTACCCCATCCGCTACAGCTTCGTAGAGTTTGTG
GAGCGGTACCGTGTGCTGCTGCCAGGTGTGAAGCCGGCCTACAAGCAGGGCGACCTCCGCGG
GACTTGCCAGCGCATGGCTGAGGCTGTGCTGGGCACCCACGATGACTGGCAGATAGGCAAAA
CCAAGATCTTTCTGAAGGACCACCATGACATGCTGCTGGAAGTGGAGCGGGACAAAGCCATC
ACCGACAGAGTCATCCTCCTTCAGAAAGTCATCCGGGGATTCAAAGACAGGTCTAACTTTCT
GAAGCTGAAGAACGCTGCCACACTGATCCAGAGGCACTGGCGGGGTCACAACTGTAGGAAGA
ACTACGGGCTGATGCGTCTGGGCTTCCTGCGGCTGCAGGCCCTGCACCGCTCCCGGAAGCTG
CACCAGCAGTACCGCCTGGCCCGCCAGCGCATCATCCAGTTCCAGGCCCGCTGCCGCGCCTA
TCTGGTGCGCAAGGCCTTCCGCCACCGCCTCTGGGCTGTGCTCACCGTGCAGGCCTATGCCC
GGGGCATGATCGCCCGCAGGCTGCACCAACGCCTCAGGGCTGAGTATCTGTGGCGCCTCGAG
GCTGAGAAAATGCGGCTGGCGGAGGAAGAGAAGCTTCGGAAGGAGATGAGCGCCAAGAAGGC
CAAGGAGGAGGCCGAGCGCAAGCATCAGGAGCGCCTGGCCCAGCTGGCTCGTGAGGACGCTG
AGCGGGAGCTGAAGGAGAAGGAGGCCGCTCGGCGGAAGAAGGAGCTCCTGGAGCAGATGGAA
AGGGCCCGCCATGAGCCTGTCAATCACTCAGACATGGTGGACAAGATGTTTGGCTTCCTGGG
GACTTCAGGTGGCCTGCCAGGCCAGGAGGGCCAGGCACCTAGTGGCTTTGAGGACCTGGAGC
GAGGGCGGAGGGAGATGGTGGAGGAGGACCTGGATGCAGCCCTGCCCCTGCCTGACGAGGAT
GAGGAGGACCTCTCTGAGTATAAATTTGCCAAGTTCGCGGCCACCTACTTCCAGGGGACAAC
CACGCACTCCTACACCCGGCGGCCACTCAAACAGCCACTGCTCTACCATGACGACGAGGGTG
ACCAGCTG
(SEQ ID NO: 40; NM_000260.3 nucleotides 273-3380)

In some embodiments, the 5′ end portion of the transgene comprises or consists of a nucleic acid sequence that has at least 70%, 80%, 90%, 95%, 96%, 97%, 98%, 99% or 100% nucleotide identity to SEQ ID NO: 40, preferably wherein the 5′ end portion of the transgene substantially retains the natural function of the 5′ end portion of the transgene of SEQ ID NO: 40.

In preferred embodiments, the 5′ end portion of the transgene comprises or consists of the nucleic acid sequence of SEQ ID NO: 40.

In preferred embodiments, the first vector comprises a 5′ end portion of the transgene that comprises or consists of the nucleic acid sequence of SEQ ID NO: 40.

An example 3′ end portion of a MYO7A transgene is:

GCAGCCCTGGCGGTCTGGATCACCATCCTCCGCTTCATGGGGGACCTCCCTGAGCCCAAGTA
CCACACAGCCATGAGTGATGGCAGTGAGAAGATCCCTGTGATGACCAAGATTTATGAGACCC
TGGGCAAGAAGACGTACAAGAGGGAGCTGCAGGCCCTGCAGGGCGAGGGCGAGGCCCAGCTC
CCCGAGGGCCAGAAGAAGAGCAGTGTGAGGCACAAGCTGGTGCATTTGACTCTGAAAAAGAA
GTCCAAGCTCACAGAGGAGGTGACCAAGAGGCTGCATGACGGGGAGTCCACAGTGCAGGGCA
ACAGCATGCTGGAGGACCGGCCCACCTCCAACCTGGAGAAGCTGCACTTCATCATCGGCAAT
GGCATCCTGCGGCCAGCACTCCGGGACGAGATCTACTGCCAGATCAGCAAGCAGCTGACCCA
CAACCCCTCCAAGAGCAGCTATGCCCGGGGCTGGATTCTCGTGTCTCTCTGCGTGGGCTGTT
TCGCCCCCTCCGAGAAGTTTGTCAAGTACCTGCGGAACTTCATCCACGGGGGCCCGCCCGGC
TACGCCCCGTACTGTGAGGAGCGCCTGAGAAGGACCTTTGTCAATGGGACACGGACACAGCC
GCCCAGCTGGCTGGAGCTGCAGGCCACCAAGTCCAAGAAGCCAATCATGTTGCCCGTGACAT
TCATGGATGGGACCACCAAGACCCTGCTGACGGACTCGGCAACCACGGCCAAGGAGCTCTGC
AACGCGCTGGCCGACAAGATCTCTCTCAAGGACCGGTTCGGGTTCTCCCTCTACATTGCCCT
GTTTGACAAGGTGTCCTCCCTGGGCAGCGGCAGTGACCACGTCATGGACGCCATCTCCCAGT
GCGAGCAGTACGCCAAGGAGCAGGGCGCCCAGGAGCGCAACGCCCCCTGGAGGCTCTTCTTC
CGCAAAGAGGTCTTCACGCCCTGGCACAGCCCCTCCGAGGACAACGTGGCCACCAACCTCAT
CTACCAGCAGGTGGTGCGAGGAGTCAAGTTTGGGGAGTACAGGTGTGAGAAGGAGGACGACC
TGGCTGAGCTGGCCTCCCAGCAGTACTTTGTAGACTATGGCTCTGAGATGATCCTGGAGCGC
CTCCTGAACCTCGTGCCCACCTACATCCCCGACCGCGAGATCACGCCCCTGAAGACGCTGGA
GAAGTGGGCCCAGCTGGCCATCGCCGCCCACAAGAAGGGGATTTATGCCCAGAGGAGAACTG
ATGCCCAGAAGGTCAAAGAGGATGTGGTCAGTTATGCCCGCTTCAAGTGGCCCTTGCTCTTC
TCCAGGTTTTATGAAGCCTACAAATTCTCAGGCCCCAGTCTCCCCAAGAACGACGTCATCGT
GGCCGTCAACTGGACGGGTGTGTACTTTGTGGATGAGCAGGAGCAGGTACTTCTGGAGCTGT
CCTTCCCAGAGATCATGGCCGTGTCCAGCAGCAGGGAGTGCCGTGTCTGGCTCTCACTGGGC
TGCTCTGATCTTGGCTGTGCTGCGCCTCACTCAGGCTGGGCAGGACTGACCCCGGCGGGGCC
CTGTTCTCCGTGTTGGTCCTGCAGGGGAGCGAAAACGACGGCCCCCAGCTTCACGCTGGCCA
CCATCAAGGGGGACGAATACACCTTCACCTCCAGCAATGCTGAGGACATTCGTGACCTGGTG
GTCACCTTCCTAGAGGGGCTCCGGAAGAGATCTAAGTATGTTGTGGCCCTGCAGGATAACCC
CAACCCCGCAGGCGAGGAGTCAGGCTTCCTCAGCTTTGCCAAGGGAGACCTCATCATCCTGG
ACCATGACACGGGCGAGCAGGTCATGAACTCGGGCTGGGCCAACGGCATCAATGAGAGGACC
AAGCAGCGTGGGGACTTCCCCACCGACAGTGTGTACGTCATGCCCACTGTCACCATGCCACC
GCGGGAGATTGTGGCCCTGGTCACCATGACTCCCGATCAGAGGCAGGACGTTGTCCGGCTCT
TGCAGCTGCGAACGGCGGAGCCCGAGGTGCGTGCCAAGCCCTACACGCTGGAGGAGTTTTCC
TATGACTACTTCAGGCCCCCACCCAAGCACACGCTGAGCCGTGTCATGGTGTCCAAGGCCCG
AGGCAAGGACCGGCTGTGGAGCCACACGCGGGAACCGCTCAAGCAGGCGCTGCTCAAGAAGC
TCCTGGGCAGTGAGGAGCTCTCGCAGGAGGCCTGCCTGGCCTTCATTGCTGTGCTCAAGTAC
ATGGGCGACTACCCGTCCAAGAGGACACGCTCCGTCAACGAGCTCACCGACCAGATCTTTGA
GGGTCCCCTGAAAGCCGAGCCCCTGAAGGACGAGGCATATGTGCAGATCCTGAAGCAGCTGA
CCGACAACCACATCAGGTACAGCGAGGAGCGGGGTTGGGAGCTGCTCTGGCTGTGCACGGGC
CTTTTCCCACCCAGCAACATCCTCCTGCCCCACGTGCAGCGCTTCCTGCAGTCCCGAAAGCA
CTGCCCACTCGCCATCGACTGCCTGCAACGGCTCCAGAAAGCCCTGAGAAACGGGTCCCGGA
AGTACCCTCCGCACCTGGTGGAGGTGGAGGCCATCCAGCACAAGACCACCCAGATTTTCCAC
AAAGTCTACTTCCCTGATGACACTGACGAGGCCTTCGAAGTGGAGTCCAGCACCAAGGCCAA
GGACTTCTGCCAGAACATCGCCACCAGGCTGCTCCTCAAGTCCTCAGAGGGATTCAGCCTCT
TTGTCAAAATTGCAGACAAGGTCCTCAGCGTTCCTGAGAATGACTTCTTCTTTGACTTTGTT
CGACACTTGACAGACTGGATAAAGAAAGCTCGGCCCATCAAGGACGGAATTGTGCCCTCACT
CACCTACCAGGTGTTCTTCATGAAGAAGCTGTGGACCACCACGGTGCCAGGGAAGGATCCCA
TGGCCGATTCCATCTTCCACTATTACCAGGAGTTGCCCAAGTATCTCCGAGGCTACCACAAG
TGCACGCGGGAGGAGGTGCTGCAGCTGGGGGCGCTGATCTACAGGGTCAAGTTCGAGGAGGA
CAAGTCCTACTTCCCCAGCATCCCCAAGCTGCTGCGGGAGCTGGTGCCCCAGGACCTTATCC
GGCAGGTCTCACCTGATGACTGGAAGCGGTCCATCGTCGCCTACTTCAACAAGCACGCAGGG
AAGTCCAAGGAGGAGGCCAAGCTGGCCTTCCTGAAGCTCATCTTCAAGTGGCCCACCTTTGG
CTCAGCCTTCTTCGAGGTGAAGCAAACTACGGAGCCAAACTTCCCTGAGATCCTCCTAATTG
CCATCAACAAGTATGGGGTCAGCCTCATCGATCCCAAAACGAAGGATATCCTCACCACTCAT
CCCTTCACCAAGATCTCCAACTGGAGCAGCGGCAACACCTACTTCCACATCACCATTGGGAA
CTTGGTGCGCGGGAGCAAACTGCTCTGCGAGACGTCACTGGGCTACAAGATGGATGACCTCC
TGACTTCCTACATTAGCCAGATGCTCACAGCCATGAGCAAACAGCGGGGCTCCAGGAGCGGC
AAGTGA
(SEQ ID NO: 41; NM_000260.3 nucleotides 3381-6920)

In some embodiments, the 3′ end portion of the transgene comprises or consists of a nucleic acid sequence that has at least 70%, 80%, 90%, 95%, 96%, 97%, 98%, 99% or 100% nucleotide identity to SEQ ID NO: 41, preferably wherein the 3′ end portion of the transgene substantially retains the natural function of the 3′ end portion of the transgene of SEQ ID NO: 41.

In preferred embodiments, the 3′ end portion of the transgene comprises or consists of the nucleic acid sequence of SEQ ID NO: 41.

In preferred embodiments, the first vector comprises a 3′ end portion of the transgene that comprises or consists of the nucleic acid sequence of SEQ ID NO: 41.

In some embodiments, the transgene is an ABCA4 transgene.

An example ABCA4 nucleotide sequence is:

(SEQ ID NO: 42)
ATGGGCTTCGTGAGACAGATACAGCTTTTGCTCTGGAAGAACTGGACCCTGCGGAAAAGGCA
AAAGATTCGCTTTGTGGTGGAACTCGTGTGGCCTTTATCTTTATTTCTGGTCTTGATCTGGT
TAAGGAATGCCAACCCGCTCTACAGCCATCATGAATGCCATTTCCCCAACAAGGCGATGCCC
TCAGCAGGAATGCTGCCGTGGCTCCAGGGGATCTTCTGCAATGTGAACAATCCCTGTTTTCA
AAGCCCCACCCCAGGAGAATCTCCTGGAATTGTGTCAAACTATAACAACTCCATCTTGGCAA
GGGTATATCGAGATTTTCAAGAACTCCTCATGAATGCACCAGAGAGCCAGCACCTTGGCCGT
ATTTGGACAGAGCTACACATCTTGTCCCAATTCATGGACACCCTCCGGACTCACCCGGAGAG
AATTGCAGGAAGAGGAATTCGAATAAGGGATATCTTGAAAGATGAAGAAACACTGAGACTAT
TTCTCATTAAAAACATCGGCCTGTCTGACTCAGTGGTCTACCTTCTGATCAACTCTCAAGTC
CGTCCAGAGCAGTTCGCTCATGGAGTCCCGGACCTGGCGCTGAAGGACATCGCCTGCAGCGA
GGCCCTCCTGGAGCGCTTCATCATCTTCAGCCAGAGACGCGGGGCAAAGACGGTGCGCTATG
CCCTGTGCTCCCTCTCCCAGGGCACCCTACAGTGGATAGAAGACACTCTGTATGCCAACGTG
GACTTCTTCAAGCTCTTCCGTGTGCTTCCCACACTCCTAGACAGCCGTTCTCAAGGTATCAA
TCTGAGATCTTGGGGAGGAATATTATCTGATATGTCACCAAGAATTCAAGAGTTTATCCATC
GGCCGAGTATGCAGGACTTGCTGTGGGTGACCAGGCCCCTCATGCAGAATGGTGGTCCAGAG
ACCTTTACAAAGCTGATGGGCATCCTGTCTGACCTCCTGTGTGGCTACCCCGAGGGAGGTGG
CTCTCGGGTGCTCTCCTTCAACTGGTATGAAGACAATAACTATAAGGCCTTTCTGGGGATTG
ACTCCACAAGGAAGGATCCTATCTATTCTTATGACAGAAGAACAACATCCTTTTGTAATGCA
TTGATCCAGAGCCTGGAGTCAAATCCTTTAACCAAAATCGCTTGGAGGGCGGCAAAGCCTTT
GCTGATGGGAAAAATCCTGTACACTCCTGATTCACCTGCAGCACGAAGGATACTGAAGAATG
CCAACTCAACTTTTGAAGAACTGGAACACGTTAGGAAGTTGGTCAAAGCCTGGGAAGAAGTA
GGGCCCCAGATCTGGTACTTCTTTGACAACAGCACACAGATGAACATGATCAGAGATACCCT
GGGGAACCCAACAGTAAAAGACTTTTTGAATAGGCAGCTTGGTGAAGAAGGTATTACTGCTG
AAGCCATCCTAAACTTCCTCTACAAGGGCCCTCGGGAAAGCCAGGCTGACGACATGGCCAAC
TTCGACTGGAGGGACATATTTAACATCACTGATCGCACCCTCCGCCTTGTCAATCAATACCT
GGAGTGCTTGGTCCTGGATAAGTTTGAAAGCTACAATGATGAAACTCAGCTCACCCAACGTG
CCCTCTCTCTACTGGAGGAAAACATGTTCTGGGCCGGAGTGGTATTCCCTGACATGTATCCC
TGGACCAGCTCTCTACCACCCCACGTGAAGTATAAGATCCGAATGGACATAGACGTGGTGGA
GAAAACCAATAAGATTAAAGACAGGTATTGGGATTCTGGTCCCAGAGCTGATCCCGTGGAAG
ATTTCCGGTACATCTGGGGCGGGTTTGCCTATCTGCAGGACATGGTTGAACAGGGGATCACA
AGGAGCCAGGTGCAGGCGGAGGCTCCAGTTGGAATCTACCTCCAGCAGATGCCCTACCCCTG
CTTCGTGGACGATTCTTTCATGATCATCCTGAACCGCTGTTTCCCTATCTTCATGGTGCTGG
CATGGATCTACTCTGTCTCCATGACTGTGAAGAGCATCGTCTTGGAGAAGGAGTTGCGACTG
AAGGAGACCTTGAAAAATCAGGGTGTCTCCAATGCAGTGATTTGGTGTACCTGGTTCCTGGA
CAGCTTCTCCATCATGTCGATGAGCATCTTCCTCCTGACGATATTCATCATGCATGGAAGAA
TCCTACATTACAGCGACCCATTCATCCTCTTCCTGTTCTTGTTGGCTTTCTCCACTGCCACC
ATCATGCTGTGCTTTCTGCTCAGCACCTTCTTCTCCAAGGCCAGTCTGGCAGCAGCCTGTAG
TGGTGTCATCTATTTCACCCTCTACCTGCCACACATCCTGTGCTTCGCCTGGCAGGACCGCA
TGACCGCTGAGCTGAAGAAGGCTGTGAGCTTACTGTCTCCGGTGGCATTTGGATTTGGCACT
GAGTACCTGGTTCGCTTTGAAGAGCAAGGCCTGGGGCTGCAGTGGAGCAACATCGGGAACAG
TCCCACGGAAGGGGACGAATTCAGCTTCCTGCTGTCCATGCAGATGATGCTCCTTGATGCTG
CTGTCTATGGCTTACTCGCTTGGTACCTTGATCAGGTGTTTCCAGGAGACTATGGAACCCCA
CTTCCTTGGTACTTTCTTCTACAAGAGTCGTATTGGCTTGGCGGTGAAGGGTGTTCAACCAG
AGAAGAAAGAGCCCTGGAAAAGACCGAGCCCCTAACAGAGGAAACGGAGGATCCAGAGCACC
CAGAAGGAATACACGACTCCTTCTTTGAACGTGAGCATCCAGGGTGGGTTCCTGGGGTATGC
GTGAAGAATCTGGTAAAGATTTTTGAGCCCTGTGGCCGGCCAGCTGTGGACCGTCTGAACAT
CACCTTCTACGAGAACCAGATCACCGCATTCCTGGGCCACAATGGAGCTGGGAAAACCACCA
CCTTGTCCATCCTGACGGGTCTGTTGCCACCAACCTCTGGGACTGTGCTCGTTGGGGGAAGG
GACATTGAAACCAGCCTGGATGCAGTCCGGCAGAGCCTTGGCATGTGTCCACAGCACAACAT
CCTGTTCCACCACCTCACGGTGGCTGAGCACATGCTGTTCTATGCCCAGCTGAAAGGAAAGT
CCCAGGAGGAGGCCCAGCTGGAGATGGAAGCCATGTTGGAGGACACAGGCCTCCACCACAAG
CGGAATGAAGAGGCTCAGGACCTATCAGGTGGCATGCAGAGAAAGCTGTCGGTTGCCATTGC
CTTTGTGGGAGATGCCAAGGTGGTGATTCTGGACGAACCCACCTCTGGGGTGGACCCTTACT
CGAGACGCTCAATCTGGGATCTGCTCCTGAAGTATCGCTCAGGCAGAACCATCATCATGTCC
ACTCACCACATGGACGAGGCCGACCTCCTTGGGGACCGCATTGCCATCATTGCCCAGGGAAG
GCTCTACTGCTCAGGCACCCCACTCTTCCTGAAGAACTGCTTTGGCACAGGCTTGTACTTAA
CCTTGGTGCGCAAGATGAAAAACATCCAGAGCCAAAGGAAAGGCAGTGAGGGGACCTGCAGC
TGCTCGTCTAAGGGTTTCTCCACCACGTGTCCAGCCCACGTCGATGACCTAACTCCAGAACA
AGTCCTGGATGGGGATGTAAATGAGCTGATGGATGTAGTTCTCCACCATGTTCCAGAGGCAA
AGCTGGTGGAGTGCATTGGTCAAGAACTTATCTTCCTTCTTCCAAATAAGAACTTCAAGCAC
AGAGCATATGCCAGCCTTTTCAGAGAGCTGGAGGAGACGCTGGCTGACCTTGGTCTCAGCAG
TTTTGGAATTTCTGACACTCCCCTGGAAGAGATTTTTCTGAAGGTCACGGAGGATTCTGATT
CAGGACCTCTGTTTGCGGGTGGCGCTCAGCAGAAAAGAGAAAACGTCAACCCCCGACACCCC
TGCTTGGGTCCCAGAGAGAAGGCTGGACAGACACCCCAGGACTCCAATGTCTGCTCCCCAGG
GGCGCCGGCTGCTCACCCAGAGGGCCAGCCTCCCCCAGAGCCAGAGTGCCCAGGCCCGCAGC
TCAACACGGGGACACAGCTGGTCCTCCAGCATGTGCAGGCGCTGCTGGTCAAGAGATTCCAA
CACACCATCCGCAGCCACAAGGACTTCCTGGCGCAGATCGTGCTCCCGGCTACCTTTGTGTT
TTTGGCTCTGATGCTTTCTATTGTTATCCCTCCTTTTGGCGAATACCCCGCTTTGACCCTTC
ACCCCTGGATATATGGGCAGCAGTACACCTTCTTCAGCATGGATGAACCAGGCAGTGAGCAG
TTCACGGTACTTGCAGACGTCCTCCTGAATAAGCCAGGCTTTGGCAACCGCTGCCTGAAGGA
AGGGTGGCTTCCGGAGTACCCCTGTGGCAACTCAACACCCTGGAAGACTCCTTCTGTGTCCC
CAAACATCACCCAGCTGTTCCAGAAGCAGAAATGGACACAGGTCAACCCTTCACCATCCTGC
AGGTGCAGCACCAGGGAGAAGCTCACCATGCTGCCAGAGTGCCCCGAGGGTGCCGGGGGCCT
CCCGCCCCCCCAGAGAACACAGCGCAGCACGGAAATTCTACAAGACCTGACGGACAGGAACA
TCTCCGACTTCTTGGTAAAAACGTATCCTGCTCTTATAAGAAGCAGCTTAAAGAGCAAATTC
TGGGTCAATGAACAGAGGTATGGAGGAATTTCCATTGGAGGAAAGCTCCCAGTCGTCCCCAT
CACGGGGGAAGCACTTGTTGGGTTTTTAAGCGACCTTGGCCGGATCATGAATGTGAGCGGGG
GCCCTATCACTAGAGAGGCCTCTAAAGAAATACCTGATTTCCTTAAACATCTAGAAACTGAA
GACAACATTAAGGTGTGGTTTAATAACAAAGGCTGGCATGCCCTGGTCAGCTTTCTCAATGT
GGCCCACAACGCCATCTTACGGGCCAGCCTGCCTAAGGACAGAAGCCCCGAGGAGTATGGAA
TCACCGTCATTAGCCAACCCCTGAACCTGACCAAGGAGCAGCTCTCAGAGATTACAGTGCTG
ACCACTTCAGTGGATGCTGTGGTTGCCATCTGCGTGATTTTCTCCATGTCCTTCGTCCCAGC
CAGCTTTGTCCTTTATTTGATCCAGGAGCGGGTGAACAAATCCAAGCACCTCCAGTTTATCA
GTGGAGTGAGCCCCACCACCTACTGGGTAACCAACTTCCTCTGGGACATCATGAATTATTCC
GTGAGTGCTGGGCTGGTGGTGGGCATCTTCATCGGGTTTCAGAAGAAAGCCTACACTTCTCC
AGAAAACCTTCCTGCCCTTGTGGCACTGCTCCTGCTGTATGGATGGGCGGTCATTCCCATGA
TGTACCCAGCATCCTTCCTGTTTGATGTCCCCAGCACAGCCTATGTGGCTTTATCTTGTGCT
AATCTGTTCATCGGCATCAACAGCAGTGCTATTACCTTCATCTTGGAATTATTTGAGAATAA
CCGGACGCTGCTCAGGTTCAACGCCGTGCTGAGGAAGCTGCTCATTGTCTTCCCCCACTTCT
GCCTGGGCCGGGGCCTCATTGACCTTGCACTGAGCCAGGCTGTGACAGATGTCTATGCCCGG
TTTGGTGAGGAGCACTCTGCAAATCCGTTCCACTGGGACCTGATTGGGAAGAACCTGTTTGC
CATGGTGGTGGAAGGGGTGGTGTACTTCCTCCTGACCCTGCTGGTCCAGCGCCACTTCTTCC
TCTCCCAATGGATTGCCGAGCCCACTAAGGAGCCCATTGTTGATGAAGATGATGATGTGGCT
GAAGAAAGACAAAGAATTATTACTGGTGGAAATAAAACTGACATCTTAAGGCTACATGAACT
AACCAAGATTTATCCAGGCACCTCCAGCCCAGCAGTGGACAGGCTGTGTGTCGGAGTTCGCC
CTGGAGAGTGCTTTGGCCTCCTGGGAGTGAATGGTGCCGGCAAAACAACCACATTCAAGATG
CTCACTGGGGACACCACAGTGACCTCAGGGGATGCCACCGTAGCAGGCAAGAGTATTTTAAC
CAATATTTCTGAAGTCCATCAAAATATGGGCTACTGTCCTCAGTTTGATGCAATCGATGAGC
TGCTCACAGGACGAGAACATCTTTACCTTTATGCCCGGCTTCGAGGTGTACCAGCAGAAGAA
ATCGAAAAGGTTGCAAACTGGAGTATTAAGAGCCTGGGCCTGACTGTCTACGCCGACTGCCT
GGCTGGCACGTACAGTGGGGGCAACAAGCGGAAACTCTCCACAGCCATCGCACTCATTGGCT
GCCCACCGCTGGTGCTGCTGGATGAGCCCACCACAGGGATGGACCCCCAGGCACGCCGCATG
CTGTGGAACGTCATCGTGAGCATCATCAGAGAAGGGAGGGCTGTGGTCCTCACATCCCACAG
CATGGAAGAATGTGAGGCACTGTGTACCCGGCTGGCCATCATGGTAAAGGGCGCCTTTCGAT
GTATGGGCACCATTCAGCATCTCAAGTCCAAATTTGGAGATGGCTATATCGTCACAATGAAG
ATCAAATCCCCGAAGGACGACCTGCTTCCTGACCTGAACCCTGTGGAGCAGTTCTTCCAGGG
GAACTTCCCAGGCAGTGTGCAGAGGGAGAGGCACTACAACATGCTCCAGTTCCAGGTCTCCT
CCTCCTCCCTGGCGAGGATCTTCCAGCTCCTCCTCTCCCACAAGGACAGCCTGCTCATCGAG
GAGTACTCAGTCACACAGACCACACTGGACCAGGTGTTTGTAAATTTTGCTAAACAGCAGAC
TGAAAGTCATGACCTCCCTCTGCACCCTCGAGCTGCTGGAGCCAGTCGACAAGCCCAGGACT
GA

An example 5′ end portion of a ABCA4 transgene is:

(SEQ ID NO: 43)
ATGGGCTTCGTGAGACAGATACAGCTTTTGCTCTGGAAGAACTGGACCCTGCGGAAAAGGCA
AAAGATTCGCTTTGTGGTGGAACTCGTGTGGCCTTTATCTTTATTTCTGGTCTTGATCTGGT
TAAGGAATGCCAACCCGCTCTACAGCCATCATGAATGCCATTTCCCCAACAAGGCGATGCCC
TCAGCAGGAATGCTGCCGTGGCTCCAGGGGATCTTCTGCAATGTGAACAATCCCTGTTTTCA
AAGCCCCACCCCAGGAGAATCTCCTGGAATTGTGTCAAACTATAACAACTCCATCTTGGCAA
GGGTATATCGAGATTTTCAAGAACTCCTCATGAATGCACCAGAGAGCCAGCACCTTGGCCGT
ATTTGGACAGAGCTACACATCTTGTCCCAATTCATGGACACCCTCCGGACTCACCCGGAGAG
AATTGCAGGAAGAGGAATTCGAATAAGGGATATCTTGAAAGATGAAGAAACACTGAGACTAT
TTCTCATTAAAAACATCGGCCTGTCTGACTCAGTGGTCTACCTTCTGATCAACTCTCAAGTC
CGTCCAGAGCAGTTCGCTCATGGAGTCCCGGACCTGGCGCTGAAGGACATCGCCTGCAGCGA
GGCCCTCCTGGAGCGCTTCATCATCTTCAGCCAGAGACGCGGGGCAAAGACGGTGCGCTATG
CCCTGTGCTCCCTCTCCCAGGGCACCCTACAGTGGATAGAAGACACTCTGTATGCCAACGTG
GACTTCTTCAAGCTCTTCCGTGTGCTTCCCACACTCCTAGACAGCCGTTCTCAAGGTATCAA
TCTGAGATCTTGGGGAGGAATATTATCTGATATGTCACCAAGAATTCAAGAGTTTATCCATC
GGCCGAGTATGCAGGACTTGCTGTGGGTGACCAGGCCCCTCATGCAGAATGGTGGTCCAGAG
ACCTTTACAAAGCTGATGGGCATCCTGTCTGACCTCCTGTGTGGCTACCCCGAGGGAGGTGG
CTCTCGGGTGCTCTCCTTCAACTGGTATGAAGACAATAACTATAAGGCCTTTCTGGGGATTG
ACTCCACAAGGAAGGATCCTATCTATTCTTATGACAGAAGAACAACATCCTTTTGTAATGCA
TTGATCCAGAGCCTGGAGTCAAATCCTTTAACCAAAATCGCTTGGAGGGCGGCAAAGCCTTT
GCTGATGGGAAAAATCCTGTACACTCCTGATTCACCTGCAGCACGAAGGATACTGAAGAATG
CCAACTCAACTTTTGAAGAACTGGAACACGTTAGGAAGTTGGTCAAAGCCTGGGAAGAAGTA
GGGCCCCAGATCTGGTACTTCTTTGACAACAGCACACAGATGAACATGATCAGAGATACCCT
GGGGAACCCAACAGTAAAAGACTTTTTGAATAGGCAGCTTGGTGAAGAAGGTATTACTGCTG
AAGCCATCCTAAACTTCCTCTACAAGGGCCCTCGGGAAAGCCAGGCTGACGACATGGCCAAC
TTCGACTGGAGGGACATATTTAACATCACTGATCGCACCCTCCGCCTTGTCAATCAATACCT
GGAGTGCTTGGTCCTGGATAAGTTTGAAAGCTACAATGATGAAACTCAGCTCACCCAACGTG
CCCTCTCTCTACTGGAGGAAAACATGTTCTGGGCCGGAGTGGTATTCCCTGACATGTATCCC
TGGACCAGCTCTCTACCACCCCACGTGAAGTATAAGATCCGAATGGACATAGACGTGGTGGA
GAAAACCAATAAGATTAAAGACAGGTATTGGGATTCTGGTCCCAGAGCTGATCCCGTGGAAG
ATTTCCGGTACATCTGGGGCGGGTTTGCCTATCTGCAGGACATGGTTGAACAGGGGATCACA
AGGAGCCAGGTGCAGGCGGAGGCTCCAGTTGGAATCTACCTCCAGCAGATGCCCTACCCCTG
CTTCGTGGACGATTCTTTCATGATCATCCTGAACCGCTGTTTCCCTATCTTCATGGTGCTGG
CATGGATCTACTCTGTCTCCATGACTGTGAAGAGCATCGTCTTGGAGAAGGAGTTGCGACTG
AAGGAGACCTTGAAAAATCAGGGTGTCTCCAATGCAGTGATTTGGTGTACCTGGTTCCTGGA
CAGCTTCTCCATCATGTCGATGAGCATCTTCCTCCTGACGATATTCATCATGCATGGAAGAA
TCCTACATTACAGCGACCCATTCATCCTCTTCCTGTTCTTGTTGGCTTTCTCCACTGCCACC
ATCATGCTGTGCTTTCTGCTCAGCACCTTCTTCTCCAAGGCCAGTCTGGCAGCAGCCTGTAG
TGGTGTCATCTATTTCACCCTCTACCTGCCACACATCCTGTGCTTCGCCTGGCAGGACCGCA
TGACCGCTGAGCTGAAGAAGGCTGTGAGCTTACTGTCTCCGGTGGCATTTGGATTTGGCACT
GAGTACCTGGTTCGCTTTGAAGAGCAAGGCCTGGGGCTGCAGTGGAGCAACATCGGGAACAG
TCCCACGGAAGGGGACGAATTCAGCTTCCTGCTGTCCATGCAGATGATGCTCCTTGATGCTG
CTGTCTATGGCTTACTCGCTTGGTACCTTGATCAGGTGTTTCCAGGAGACTATGGAACCCCA
CTTCCTTGGTACTTTCTTCTACAAGAGTCGTATTGGCTTGGCGGTGAAGGGTGTTCAACCAG
AGAAGAAAGAGCCCTGGAAAAGACCGAGCCCCTAACAGAGGAAACGGAGGATCCAGAGCACC
CAGAAGGAATACACGACTCCTTCTTTGAACGTGAGCATCCAGGGTGGGTTCCTGGGGTATGC
GTGAAGAATCTGGTAAAGATTTTTGAGCCCTGTGGCCGGCCAGCTGTGGACCGTCTGAACAT
CACCTTCTACGAGAACCAGATCACCGCATTCCTGGGCCACAATGGAGCTGGGAAAACCACCA
CCTT

In some embodiments, the 5′ end portion of the transgene comprises or consists of a nucleic acid sequence that has at least 70%, 80%, 90%, 95%, 96%, 97%, 98%, 99% or 100% nucleotide identity to SEQ ID NO: 43, preferably wherein the 5′ end portion of the transgene substantially retains the natural function of the 5′ end portion of the transgene of SEQ ID NO: 43.

An example 3′ end portion of a ABCA4 transgene is:

(SEQ ID NO: 44)
GTCCATCCTGACGGGTCTGTTGCCACCAACCTCTGGGACTGTGCTCGTTGGGGGAAGGGACA
TTGAAACCAGCCTGGATGCAGTCCGGCAGAGCCTTGGCATGTGTCCACAGCACAACATCCTG
TTCCACCACCTCACGGTGGCTGAGCACATGCTGTTCTATGCCCAGCTGAAAGGAAAGTCCCA
GGAGGAGGCCCAGCTGGAGATGGAAGCCATGTTGGAGGACACAGGCCTCCACCACAAGCGGA
ATGAAGAGGCTCAGGACCTATCAGGTGGCATGCAGAGAAAGCTGTCGGTTGCCATTGCCTTT
GTGGGAGATGCCAAGGTGGTGATTCTGGACGAACCCACCTCTGGGGTGGACCCTTACTCGAG
ACGCTCAATCTGGGATCTGCTCCTGAAGTATCGCTCAGGCAGAACCATCATCATGTCCACTC
ACCACATGGACGAGGCCGACCTCCTTGGGGACCGCATTGCCATCATTGCCCAGGGAAGGCTC
TACTGCTCAGGCACCCCACTCTTCCTGAAGAACTGCTTTGGCACAGGCTTGTACTTAACCTT
GGTGCGCAAGATGAAAAACATCCAGAGCCAAAGGAAAGGCAGTGAGGGGACCTGCAGCTGCT
CGTCTAAGGGTTTCTCCACCACGTGTCCAGCCCACGTCGATGACCTAACTCCAGAACAAGTC
CTGGATGGGGATGTAAATGAGCTGATGGATGTAGTTCTCCACCATGTTCCAGAGGCAAAGCT
GGTGGAGTGCATTGGTCAAGAACTTATCTTCCTTCTTCCAAATAAGAACTTCAAGCACAGAG
CATATGCCAGCCTTTTCAGAGAGCTGGAGGAGACGCTGGCTGACCTTGGTCTCAGCAGTTTT
GGAATTTCTGACACTCCCCTGGAAGAGATTTTTCTGAAGGTCACGGAGGATTCTGATTCAGG
ACCTCTGTTTGCGGGTGGCGCTCAGCAGAAAAGAGAAAACGTCAACCCCCGACACCCCTGCT
TGGGTCCCAGAGAGAAGGCTGGACAGACACCCCAGGACTCCAATGTCTGCTCCCCAGGGGCG
CCGGCTGCTCACCCAGAGGGCCAGCCTCCCCCAGAGCCAGAGTGCCCAGGCCCGCAGCTCAA
CACGGGGACACAGCTGGTCCTCCAGCATGTGCAGGCGCTGCTGGTCAAGAGATTCCAACACA
CCATCCGCAGCCACAAGGACTTCCTGGCGCAGATCGTGCTCCCGGCTACCTTTGTGTTTTTG
GCTCTGATGCTTTCTATTGTTATCCCTCCTTTTGGCGAATACCCCGCTTTGACCCTTCACCC
CTGGATATATGGGCAGCAGTACACCTTCTTCAGCATGGATGAACCAGGCAGTGAGCAGTTCA
CGGTACTTGCAGACGTCCTCCTGAATAAGCCAGGCTTTGGCAACCGCTGCCTGAAGGAAGGG
TGGCTTCCGGAGTACCCCTGTGGCAACTCAACACCCTGGAAGACTCCTTCTGTGTCCCCAAA
CATCACCCAGCTGTTCCAGAAGCAGAAATGGACACAGGTCAACCCTTCACCATCCTGCAGGT
GCAGCACCAGGGAGAAGCTCACCATGCTGCCAGAGTGCCCCGAGGGTGCCGGGGGCCTCCCG
CCCCCCCAGAGAACACAGCGCAGCACGGAAATTCTACAAGACCTGACGGACAGGAACATCTC
CGACTTCTTGGTAAAAACGTATCCTGCTCTTATAAGAAGCAGCTTAAAGAGCAAATTCTGGG
TCAATGAACAGAGGTATGGAGGAATTTCCATTGGAGGAAAGCTCCCAGTCGTCCCCATCACG
GGGGAAGCACTTGTTGGGTTTTTAAGCGACCTTGGCCGGATCATGAATGTGAGCGGGGGCCC
TATCACTAGAGAGGCCTCTAAAGAAATACCTGATTTCCTTAAACATCTAGAAACTGAAGACA
ACATTAAGGTGTGGTTTAATAACAAAGGCTGGCATGCCCTGGTCAGCTTTCTCAATGTGGCC
CACAACGCCATCTTACGGGCCAGCCTGCCTAAGGACAGAAGCCCCGAGGAGTATGGAATCAC
CGTCATTAGCCAACCCCTGAACCTGACCAAGGAGCAGCTCTCAGAGATTACAGTGCTGACCA
CTTCAGTGGATGCTGTGGTTGCCATCTGCGTGATTTTCTCCATGTCCTTCGTCCCAGCCAGC
TTTGTCCTTTATTTGATCCAGGAGCGGGTGAACAAATCCAAGCACCTCCAGTTTATCAGTGG
AGTGAGCCCCACCACCTACTGGGTAACCAACTTCCTCTGGGACATCATGAATTATTCCGTGA
GTGCTGGGCTGGTGGTGGGCATCTTCATCGGGTTTCAGAAGAAAGCCTACACTTCTCCAGAA
AACCTTCCTGCCCTTGTGGCACTGCTCCTGCTGTATGGATGGGCGGTCATTCCCATGATGTA
CCCAGCATCCTTCCTGTTTGATGTCCCCAGCACAGCCTATGTGGCTTTATCTTGTGCTAATC
TGTTCATCGGCATCAACAGCAGTGCTATTACCTTCATCTTGGAATTATTTGAGAATAACCGG
ACGCTGCTCAGGTTCAACGCCGTGCTGAGGAAGCTGCTCATTGTCTTCCCCCACTTCTGCCT
GGGCCGGGGCCTCATTGACCTTGCACTGAGCCAGGCTGTGACAGATGTCTATGCCCGGTTTG
GTGAGGAGCACTCTGCAAATCCGTTCCACTGGGACCTGATTGGGAAGAACCTGTTTGCCATG
GTGGTGGAAGGGGTGGTGTACTTCCTCCTGACCCTGCTGGTCCAGCGCCACTTCTTCCTCTC
CCAATGGATTGCCGAGCCCACTAAGGAGCCCATTGTTGATGAAGATGATGATGTGGCTGAAG
AAAGACAAAGAATTATTACTGGTGGAAATAAAACTGACATCTTAAGGCTACATGAACTAACC
AAGATTTATCCAGGCACCTCCAGCCCAGCAGTGGACAGGCTGTGTGTCGGAGTTCGCCCTGG
AGAGTGCTTTGGCCTCCTGGGAGTGAATGGTGCCGGCAAAACAACCACATTCAAGATGCTCA
CTGGGGACACCACAGTGACCTCAGGGGATGCCACCGTAGCAGGCAAGAGTATTTTAACCAAT
ATTTCTGAAGTCCATCAAAATATGGGCTACTGTCCTCAGTTTGATGCAATCGATGAGCTGCT
CACAGGACGAGAACATCTTTACCTTTATGCCCGGCTTCGAGGTGTACCAGCAGAAGAAATCG
AAAAGGTTGCAAACTGGAGTATTAAGAGCCTGGGCCTGACTGTCTACGCCGACTGCCTGGCT
GGCACGTACAGTGGGGGCAACAAGCGGAAACTCTCCACAGCCATCGCACTCATTGGCTGCCC
ACCGCTGGTGCTGCTGGATGAGCCCACCACAGGGATGGACCCCCAGGCACGCCGCATGCTGT
GGAACGTCATCGTGAGCATCATCAGAGAAGGGAGGGCTGTGGTCCTCACATCCCACAGCATG
GAAGAATGTGAGGCACTGTGTACCCGGCTGGCCATCATGGTAAAGGGCGCCTTTCGATGTAT
GGGCACCATTCAGCATCTCAAGTCCAAATTTGGAGATGGCTATATCGTCACAATGAAGATCA
AATCCCCGAAGGACGACCTGCTTCCTGACCTGAACCCTGTGGAGCAGTTCTTCCAGGGGAAC
TTCCCAGGCAGTGTGCAGAGGGAGAGGCACTACAACATGCTCCAGTTCCAGGTCTCCTCCTC
CTCCCTGGCGAGGATCTTCCAGCTCCTCCTCTCCCACAAGGACAGCCTGCTCATCGAGGAGT
ACTCAGTCACACAGACCACACTGGACCAGGTGTTTGTAAATTTTGCTAAACAGCAGACTGAA
AGTCATGACCTCCCTCTGCACCCTCGAGCTGCTGGAGCCAGTCGACAAGCCCAGGACTGA

In some embodiments, the 3′ end portion of the transgene comprises or consists of a nucleic acid sequence that has at least 70%, 80%, 90%, 95%, 96%, 97%, 98%, 99% or 100% nucleotide identity to SEQ ID NO: 44, preferably wherein the 5′ end portion of the transgene substantially retains the natural function of the 5′ end portion of the transgene of SEQ ID NO: 44.

The polynucleotides used in the invention may be codon-optimised. In some embodiments, the transgene is codon optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

Exemplary Vectors

An example sequence of the first vector of the invention is:

(SEQ ID NO: 14)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG
ATCCTTATCGGGAATTCGCCCTTAAGCTAGCGTGCCACCTGGTCGACATTGATTATTGACTA

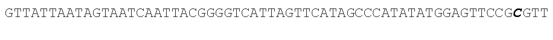

ACATAACTTACGGTAAATGGCCCGCCTGGCTGACCGCCCAACGACCCCCGCCCATTGACGTC
AATAATGACGTATGTTCCCATAGTAACGCCAATAGGGACTTTCCATTGACGTCAATGGGTGG
AGTATTTACGGTAAACTGCCCACTTGGCAGTACATCAAGTGTATCATATGCCAAGTACGCCC
CCTATTGACGTCAATGACGGTAAATGGCCCGCCTGGCATTATGCCCAGTACATGACCTTATG
GGACTTTCCTACTTGGCAGTACATCTACGTATTAGTCATCGCTATTACCATGGTCGAGGTGA
GCCCCACGTTCTGCTTCACTCTCCCCATCTCCCCCCCCTCCCCACCCCCAATTTTGTATTTA
TTTATTTTTTAATTATTTTGTGCAGCGATGGGGGCGGGGCGGGGCGAGGCGGAGAGGTGCGG
CGGCAGCCAATCGGAGCGGCGCGCTCCGAAAGTTTCCTTTTATGGCGAGGCGGCGGCGGCGG
CGGCTCTATAAAAAGCGAAGCGCGCGGCGGGCGGCTGCAGAAGTTGGTCGTGAGGCACTGGG
CAGCTCTAAGGTAAATATAAAATTTTTAAGTGTATAATGTGTTAAACTACTGATTCTAATTG

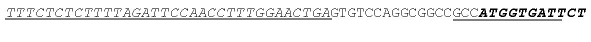

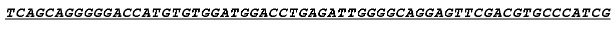

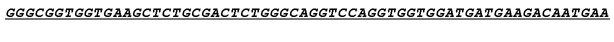

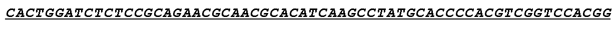

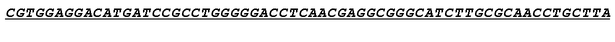

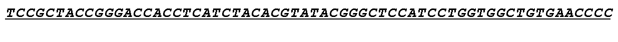

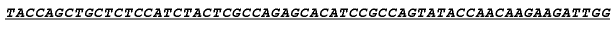

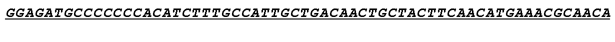

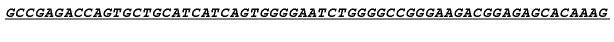

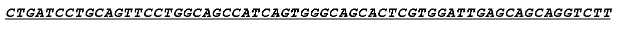

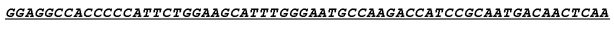

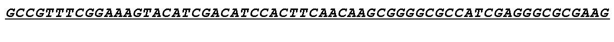

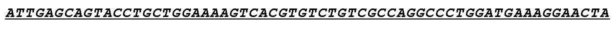

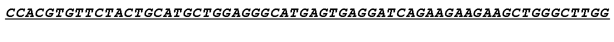

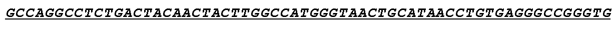

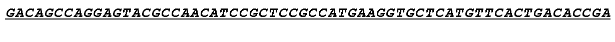

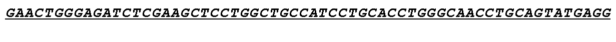

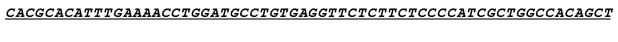

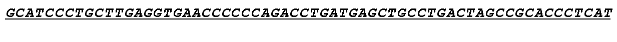

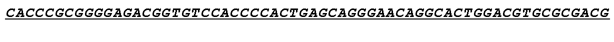

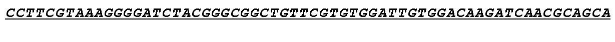

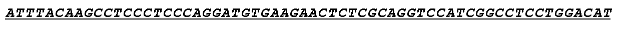

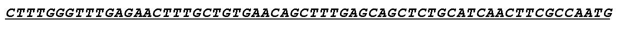

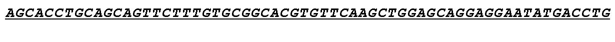

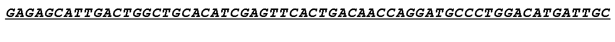

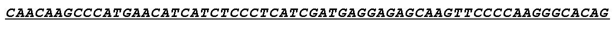

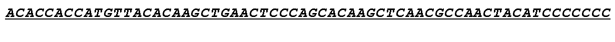

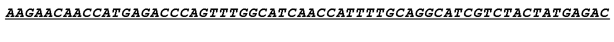

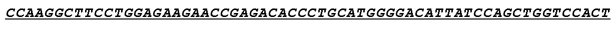

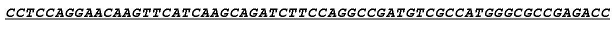

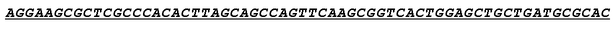

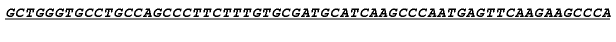

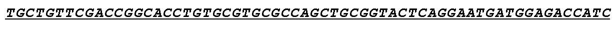

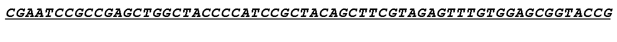

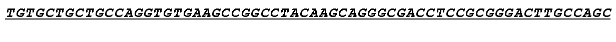

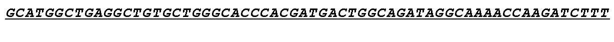

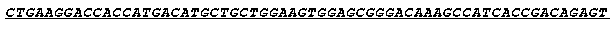

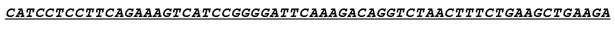

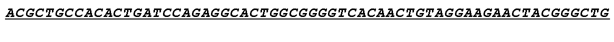

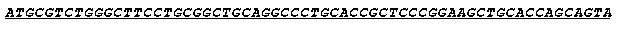

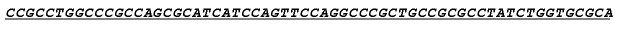

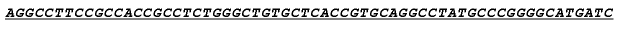

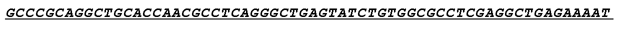

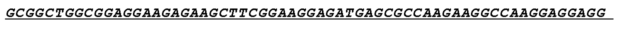

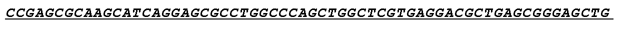

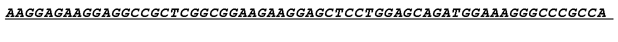

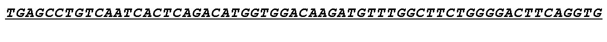

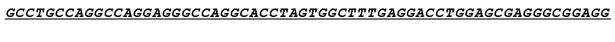

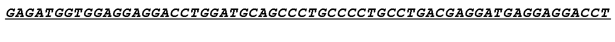

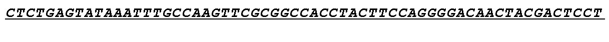

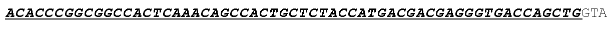

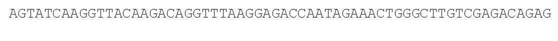

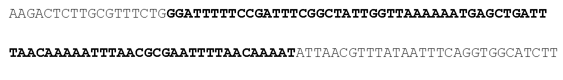

TCCAATTGAAGGGCGAATTCCGATCTTCCTAGAGCATGGCTACGTAGATAAGTAGCATGGCG
GGTTAATCATTAACTACAAGGAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGGC
CTCAGTGAGCGAGCGAGCGCGCAG
5’ ITR
CMV enhancer
CBA promoter
Modified SV40 intron

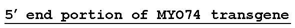

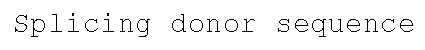

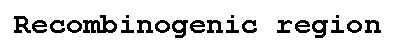

3’ ITR

In some embodiments, the first vector comprises or consists of a nucleic acid sequence that has at least 70%, 80%, 90%, 95%, 96%, 97%, 98%, 99% or 100% nucleotide identity to SEQ ID NO: 14, preferably wherein the first vector substantially retains the natural function of the first vector of SEQ ID NO: 14.

In preferred embodiments, the first vector comprises or consists of the nucleic acid sequence of SEQ ID NO: 14.

An example sequence of the second vector of the invention is:

(SEQ ID NO: 15)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG

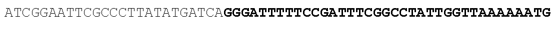

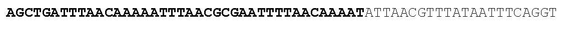

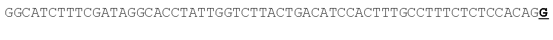

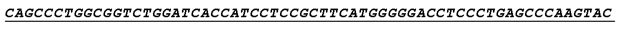

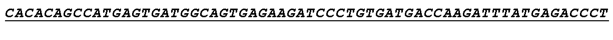

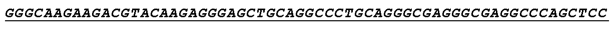

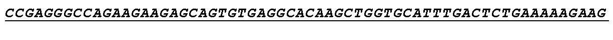

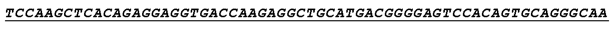

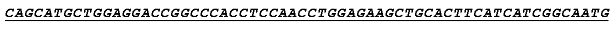

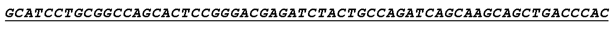

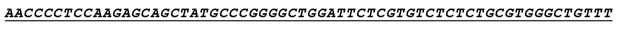

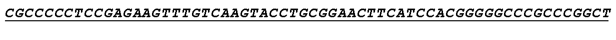

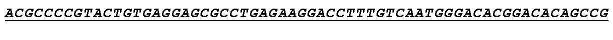

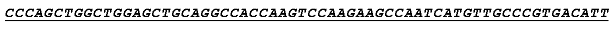

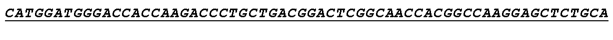

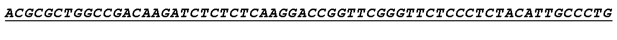

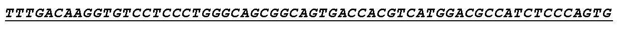

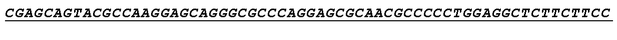

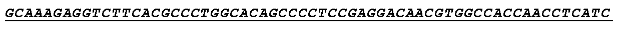

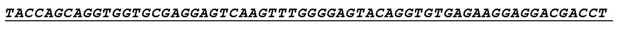

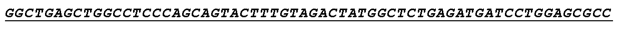

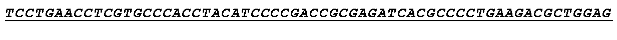

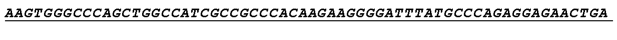

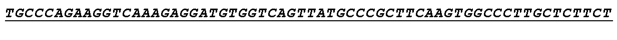

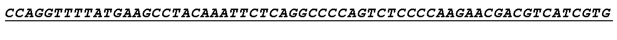

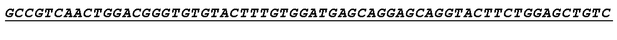

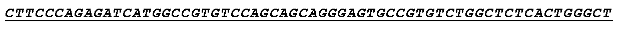

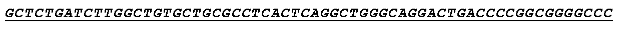

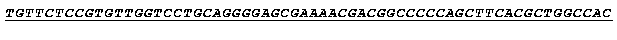

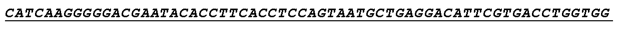

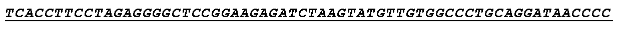

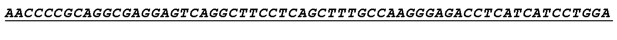

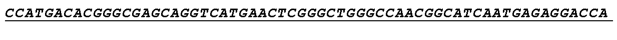

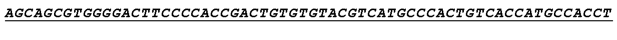

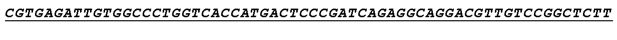

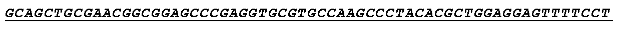

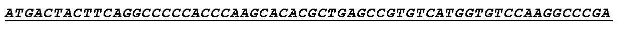

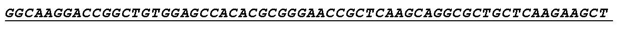

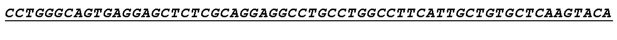

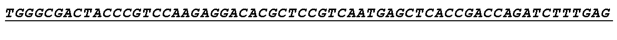

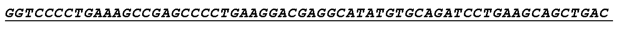

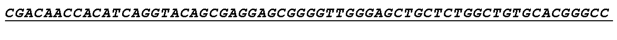

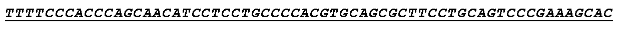

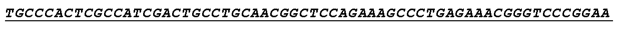

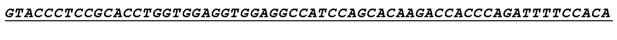

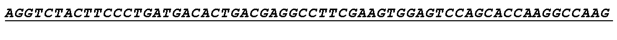

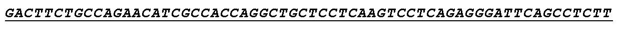

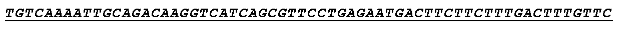

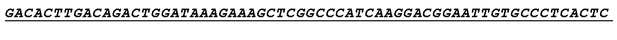

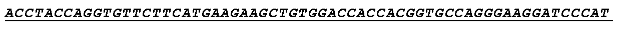

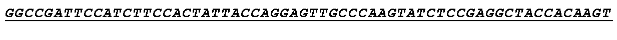

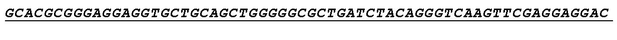

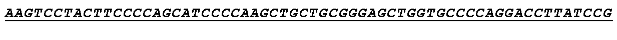

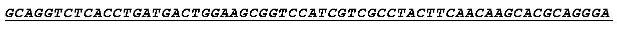

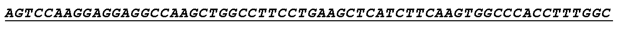

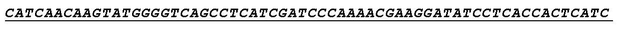

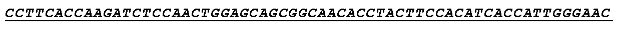

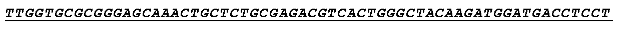

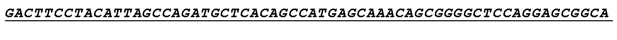

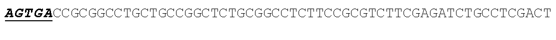

GTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGGGAAGAC
AATAGCAGGCATGCTGGGGACTCGAGCAATTCCCGATAAGGATCTTCCTAGAGCATGGCTAC
GTAGATAAGTAGCATGGCGGGTTAATCATTAACTACAAGGAACCCCTAGTGATGGAGTTGGC
CACTCCCTCTCTGCGCGCTCGCTCGCTCACTGAGGCCGGGCGACCAAAGGTCGCCCGACGCC
CGGGCTTTGCCCGGGCGGCCTCAGTGAGCGAGCGAGCGCGCAG
5’ ITR

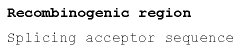

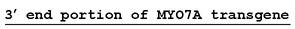

bGH polyadenylation sequence
3’ ITR

In some embodiments, the second vector comprises or consists of a nucleic acid sequence that has at least 70%, 80%, 90%, 95%, 96%, 97%, 98%, 99% or 100% nucleotide identity to SEQ ID NO: 15, preferably wherein the second vector substantially retains the natural function of the second vector of SEQ ID NO: 15.

In preferred embodiments, the second vector comprises or consists of the nucleic acid sequence of SEQ ID NO: 15.

In particularly preferred embodiments, the first vector comprises or consists of the nucleic acid sequence of SEQ ID NO: 14 and the second vector comprises or consists of the nucleic acid sequence of SEQ ID NO: 15.

Compositions

The vectors, vector systems and cells of the invention may be formulated for administration to subjects with a pharmaceutically-acceptable carrier, diluent or excipient. Suitable carriers and diluents include isotonic saline solutions, for example phosphate-buffered saline, and potentially contain human serum albumin.

Materials used to formulate a pharmaceutical composition should be non-toxic and should not interfere with the efficacy of the active ingredient. The precise nature of the carrier or other material may be determined by the skilled person according to the route of administration.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

For injection, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

Method of Treatment

In one aspect the invention provides the vector system, vector, kit or composition of the invention for use in therapy.

In another aspect the invention provides the vector system, vector, kit or composition of the invention for use in treatment of a retinal degeneration. Preferably the retinal degeneration is an inherited retinal degeneration.

In some embodiments, the use is in treatment or prevention of Usher syndrome, retinitis pigmentosa, Leber congenital amaurosis (LCA), Stargardt disease, Alstrom syndrome or ABCA4-associated diseases.

In another aspect the invention provides the vector system, vector, kit or composition of the invention for use in treatment of Usher syndrome.

In preferred embodiments, the Usher syndrome is Usher syndrome Type 1B.

In another aspect the invention provides a method of treating or preventing a retinal degeneration comprising administering an effective amount of the vector system, vector, kit or composition of the invention to a subject in need thereof. Preferably the retinal degeneration is an inherited retinal degeneration.

In another aspect the invention provides a method of treating or preventing Usher syndrome comprising administering an effective amount of the vector system, vector, kit or composition of the invention to a subject in need thereof.

In some embodiments, localisation of melanosomes to the retinal pigment epithelium (RPE) apical villi is increased or normalised (e.g. increased to a level that is about the same as that of a healthy subject). The increase may be in comparison to RPE apical villi from an eye that has not been treated in accordance with the invention (for example, is an eye from a subject with the disease but under otherwise substantially the same conditions). The increase (e.g. in the number per 100 μm) may, for example, be an increase of at least 2-fold, at least 3-fold, at least 4-fold, at least 5-fold, at least 6-fold, at least 7-fold, at least 8-fold, at least 9-fold or at least 10-fold. The increase may, for example, increase the number of melanosomes (e.g. the number per 100 μm) to within 30%, 40%, 50%, 60%, 70%, 80%, 90%, 95% or 99% of the number for a healthy subject. Methods for analysing melanosomes are well known to the skilled person and include, for example, methods disclosed herein.

Inherited retinal degenerations (IRDs), with an overall global prevalence of 1/2,000, are a major cause of blindness worldwide. Among the most frequent and severe IRDs are retinitis pigmentosa (RP), Leber congenital amaurosis (LCA) and Stargardt disease (STGD), which are most often inherited as monogenic conditions. The majority of mutations causing IRDs occur in genes expressed in neuronal photoreceptors (PR), rods and/or cones in the retina.

AAV vectors are among the most efficient vectors at targeting both PR and retinal pigment epithelium (RPE) for long-term treatment upon a single subretinal administration. The invention enables the treatment of disease such as those listed in the table below, which may be difficult to treat with single AAV vectors (which may have a maximum cargo capacity of about 5 kb):

| DISEASE | GENE | CDS | EXPRESSION |
|---|---|---|---|
| Usher 1B | MYO7A | 6.7 Kb | RPE and PRs |
| Stargardt Disease | ABCA4 | 6.8 Kb | Rod & cone PRs |
| Leber Congenital | CEP290 | 7.5 Kb | Mainly PRs (pan retinal) |
| Amaurosis | | | |
| Usher1D, Nonsyndromic | CDH23 | 10.1 Kb | PRs |
| deafness, autosomal | | | |
| recessive (DFNB12) | | | |
| Retinitis Pigmentosa | EYS | 9.4 Kb | PR ECM |
| Usher 2A | USH2a | 15.6 Kb | Rod & cone PRs |
| Usher 2C | GPR98 | 18.0 Kb | Mainly PRs |
| Alstrom Syndrome | ALMS1 | 12.5 Kb | Rod & cone PRs |

Usher syndrome type IB (USH1B) is the most severe form of RP and deafness caused by mutations in the MYO7A gene (CDS: 6648 bp) encoding the unconventional MYO7A, an actin-based motor expressed in both PR and RPE within the retina.

Stargardt disease (STGD) is the most common form of inherited macular degeneration caused by mutations in the ABCA4 gene (CDS: 6822 bp), which encodes the all-trans retinal transporter located in the PR outer segment.

Cone-rod dystrophy type 3, fundus flavimaculatus, age-related macular degeneration type 2, Early-onset severe retinal dystrophy and Retinitis pigmentosa type 19 are also associated with ABCA4 mutations (ABCA4-associated diseases).

All references herein to treatment include curative, palliative and prophylactic treatment. The treatment of mammals, particularly humans, is preferred. Both human and veterinary treatments are within the scope of the invention.

Administration

In some embodiments, the vectors, vector systems or cells are administered to a subject locally.

In some embodiments, the vectors, vector systems or cells are administered to a subject's eye. The administration may be by injection, for example subretinal injection.

The first vector and the second vector may be administered in combination simultaneously, sequentially or separately.

The term “combination”, or terms “in combination”, “used in combination with” or “combined preparation” as used herein may refer to the combined administration of two or more agents simultaneously, sequentially or separately.

The term “simultaneous” as used herein means that the agents are administered concurrently, i.e. at the same time.

The term “sequential” as used herein means that the agents are administered one after the other.

The term “separate” as used herein means that the agents are administered independently of each other but within a time interval that allows the agents to show a combined, preferably synergistic, effect. Thus, administration “separately” may permit one agent to be administered, for example, within 1 minute, 5 minutes or 10 minutes after the other.

Dosage

The skilled person can readily determine an appropriate dose of an agent of the invention to administer to a subject. Typically, a physician will determine the actual dosage which will be most suitable for an individual patient, and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

The dose may, for example, be sufficient to treat or prevent the retinal degeneration. The dose may, for example, be sufficient to treat or prevent the Usher syndrome, retinitis pigmentosa, Leber congenital amaurosis (LCA), Stargardt disease, Alstrom syndrome or ABCA4-associated diseases.

In some embodiments, the dose is 1×10⁹ to 1.5×10¹⁰ total genome copies per eye. In some embodiments, the dose is 4×10⁹ to 1.5×10¹⁰ total genome copies per eye. In some embodiments, the dose is 1×10⁹ to 8×10⁹ total genome copies per eye, 2×10⁹ to 7×10⁹ total genome copies per eye, 3×10⁹ to 6×10⁹ total genome copies per eye or 4×10⁹ to 5×10⁹ total genome copies per eye. In some embodiments, the dose is 7×10⁹ to 5×10¹⁰ total genome copies per eye, 8×10⁹ to 4×10¹⁰ total genome copies per eye, 9×10⁹ to 3×10¹⁰ total genome copies per eye or 1×10¹⁰ to 2×10¹⁰ total genome copies per eye.

An equivalent dose may be used that is optimised for a human subject. In some embodiments, the dose is 1×10⁹ to 2×10¹² total genome copies per eye. In some embodiments, the dose is 1×10¹⁰ to 2×10¹² total genome copies per eye. In some embodiments, the dose is 1×10¹¹ to 2×10¹² total genome copies per eye.

In some embodiments, the dose is 1×10¹¹ to 1.5×10¹² total genome copies per eye. In some embodiments, the dose is 4×10¹¹ to 1.5×10¹² total genome copies per eye. In some embodiments, the dose is 1×10¹¹ to 8×10¹¹ total genome copies per eye, 2×10¹¹ to 7×10¹¹ total genome copies per eye, 3×10¹¹ to 6×10¹¹ total genome copies per eye or 4×10¹¹ to 5×10¹¹ total genome copies per eye. In some embodiments, the dose is 7×10¹¹ to 5×10¹² total genome copies per eye, 8×10¹¹ to 4×10¹² total genome copies per eye, 9×10¹¹ to 3×10¹² total genome copies per eye or 1×10¹¹ to 2×10¹² total genome copies per eye.

An equivalent dose may be used that is optimised for a different non-human subject.

Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is a human.

Variants, Derivatives, Analogues, and Fragments

In addition to the specific proteins and polynucleotides mentioned herein, the invention also encompasses variants, derivatives and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

Typically, a variant may have a certain identity with the wild type amino acid sequence or the wild type nucleotide sequence.

In the present context, a variant sequence is taken to include an amino acid sequence which may be at least 50%, 55%, 65%, 75%, 85% or 90% identical, suitably at least 95%, 96% or 97% or 98% or 99% identical to the subject sequence. Although a variant can also be considered in terms of similarity (i.e. amino acid residues having similar chemical properties/functions), in the context of the present invention it is preferred to express in terms of sequence identity.

In the present context, a variant sequence is taken to include a nucleotide sequence which may be at least 50%, 55%, 65%, 75%, 85% or 90% identical, suitably at least 95%, 96% or 97% or 98% or 99% identical to the subject sequence. Although a variant can also be considered in terms of similarity, in the context of the present invention it is preferred to express it in terms of sequence identity.

Suitably, reference to a sequence which has a percent identity to any one of the SEQ ID NOs detailed herein refers to a sequence which has the stated percent identity over the entire length of the SEQ ID NO referred to.

Sequence identity comparisons can be conducted by eye, or more usually, with the aid of readily available sequence comparison programs. These commercially available computer programs can calculate percent identity between two or more sequences.

Percent identity may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent identity when a global alignment is performed. Consequently, most sequence comparison methods are designed to produce optimal alignments that take into consideration possible insertions and deletions without penalising unduly the overall identity score. This is achieved by inserting “gaps” in the sequence alignment to try to maximise local identity.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent identity therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410), EMBOSS Needle (Madeira, F., et al., 2019. Nucleic acids research, 47(W1), pp. W636-W641) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174(2):247-50; FEMS Microbiol. Lett. (1999) 177(1):187-8).

Although the final percent identity can be measured, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent sequence identity. The software typically does this as part of the sequence comparison and generates a numerical result. The percent sequence identity may be calculated as the number of identical residues as a percentage of the total residues in the SEQ ID NO referred to.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

Results and Discussion

Optimisation of Dual Adeno-Associated Viral (AAV) Vectors

During the characterisation of dual AAV8 vectors for the delivery of human Myosin7A (hMYO7A), we discovered a contaminant vector in preparations of the vector comprising the 5′ end portion of the transgene coding sequence CDS (AAV8-5′hMYO7A).

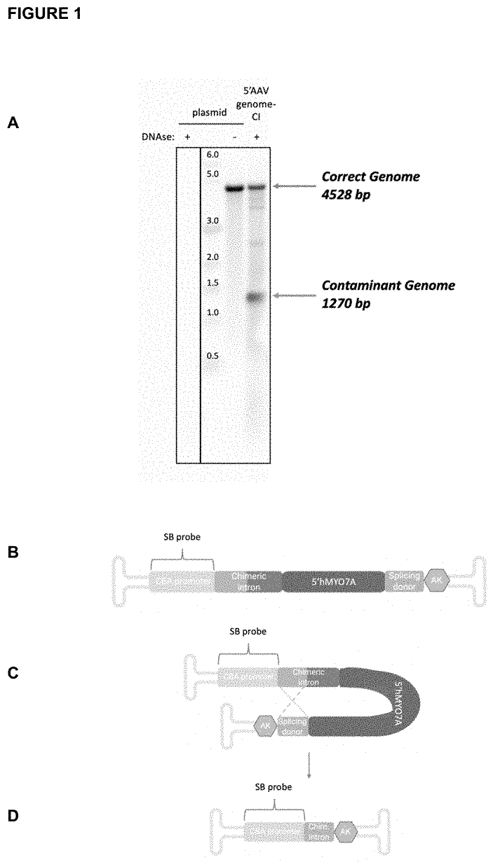

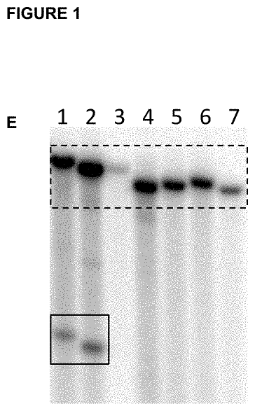

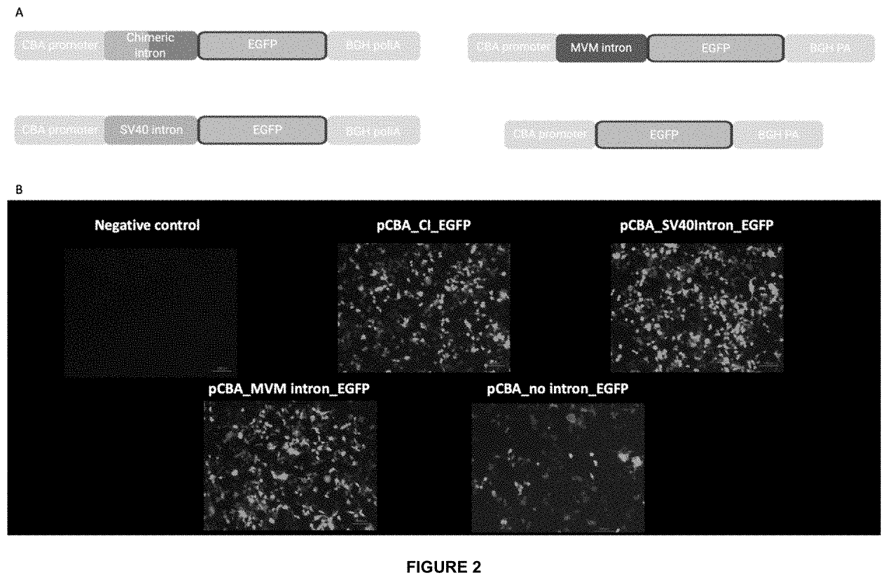

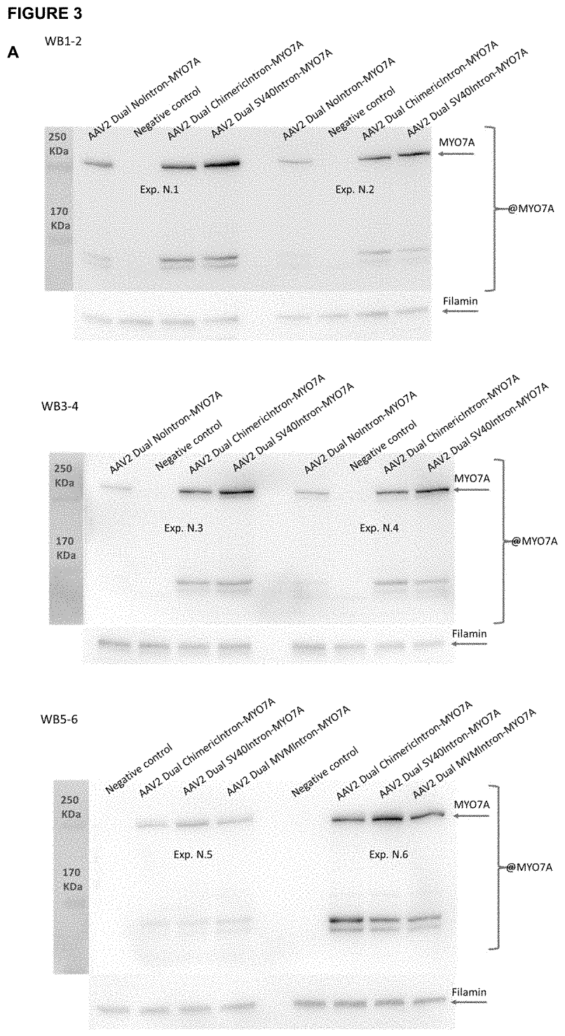

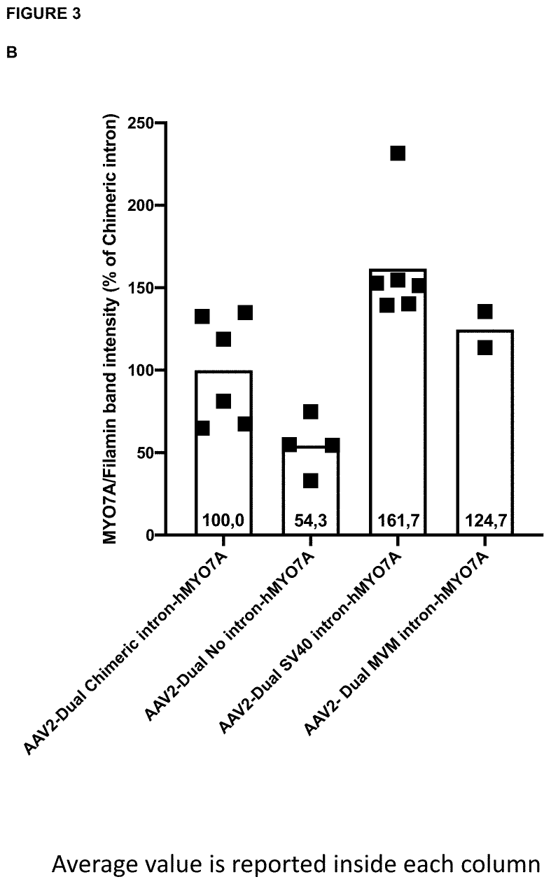

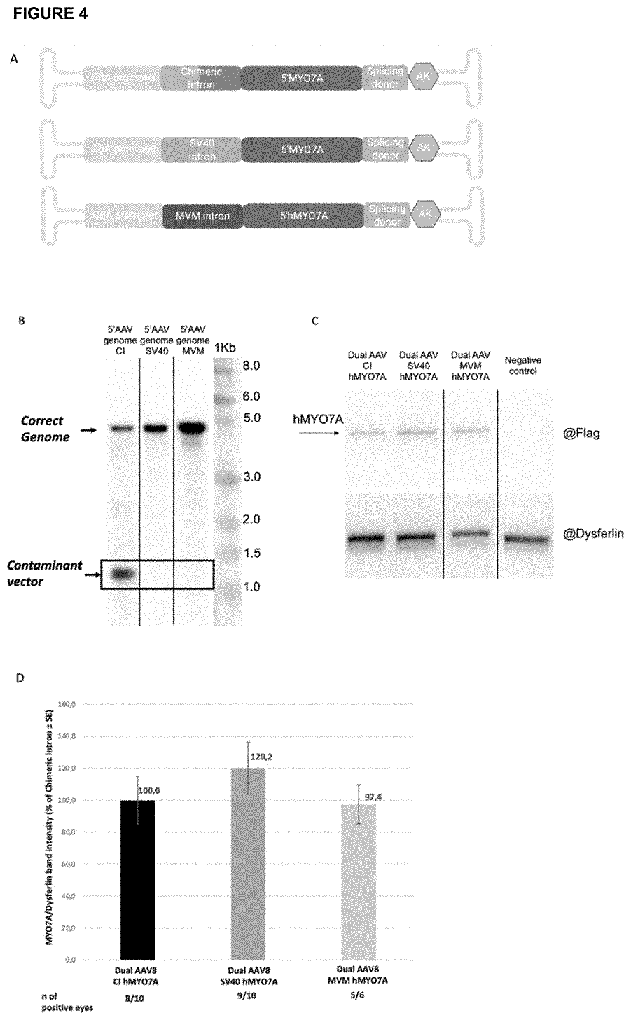

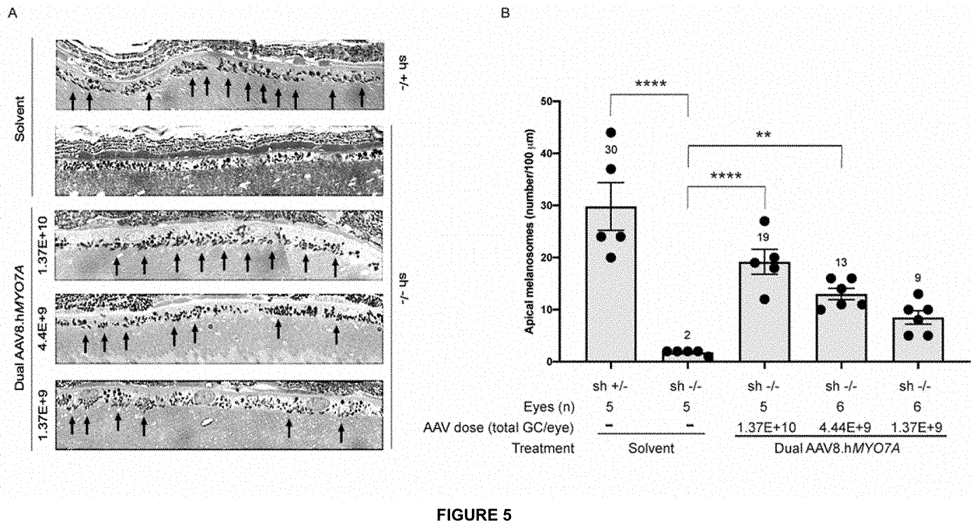

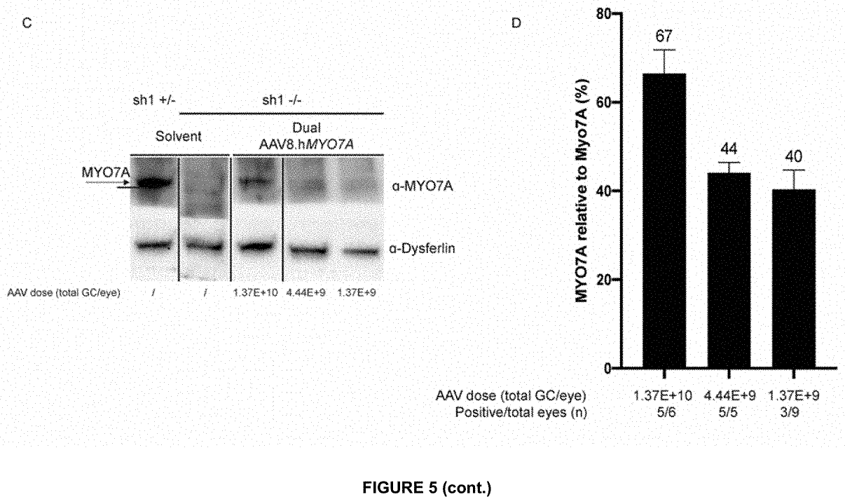

Southern blot analysis, developed using a probe that recognises the chicken beta-actin (CBA) promoter used in the vector, showed a larger band corresponding to the expected AAV8-5′hMYO7A and a smaller band of about 1.3 Kb corresponding to the contaminant (FIG. 1A, B).

The smaller genome contaminant was consistently present in the vector preparations, yet absent in the plasmid used to generate them. Accordingly, we hypothesised that the problem was related to the viral genome and that the generation of the smaller product occurred upon or after manufacturing of the vector particle since the original plasmid genome was clearly intact.

We then identified an 82 base pair homology region between two sequences: the chimeric promoter intron and the splicing donor (SD) signal (FIG. 1C, see sequences below).
Chimeric intron (Bothwell et al. (1981) Cell 24: 625-637):

(SEQ ID NO: 16)
GTAAGTATCAAGGTTACAAGACAGGTTTAAGGAGACCAATAGAAACTGG
GCTTGTCGAGACAGAGAAGACTCTTGCGTTTCTGATAGGCACCTATTGG
TCTTACTGACATCCACTTTGCCTTTCTCTCCACAG

Splicing donor (SD) sequence:

(SEQ ID NO: 17)
GTAAGTATCAAGGTTACAAGACAGGTTTAAGGAGACCAATAGAAACTGG
GCTTGTCGAGACAGAGAAGACTCTTGCGTTTCT

The underlined sequences are identical: the SD sequence is identical to nucleotides 1-82 of the chimeric intron.

Using subcloning and Sanger sequencing of the purified viral DNA, we confirmed that a homologous recombination event takes place due to the presence of the regions of homology within the construct. This leads to the deletion of the remaining portion of the intron, the 5′hMYO7A sequence and the SD signal while the new construct still retains AAV inverted terminal repeats (ITRs), thus supporting vector production (FIG. 1D).

Similar contaminants were observed in other vectors (e.g. comprising other transgenes and promoters) containing the intron sequence and SD sequence (FIG. 1E), and were abolished by removing the intron sequence.

We then substituted the chimeric intron with a sequence that was not homologous to the SD sequence. We cloned plasmids encoding for Enhanced Green Fluorescent Protein (EGFP) with either the chimeric intron, a modified version of the simian virus 40 (SV40) intron (Nathwani et al. (2006) Blood 107: 2653-2661), the minute virus mice (MVM) intron (Wu et al. (2008) Mol. Ther. 16: 280-289) or no intron, in order to make a comparison in terms of EGFP expression in HEK293 cells by transfection. Fluorescence imaging (FIG. 2B) shows that EGFP expression from the constructs containing the SV40 intron or the MVM intron is similar to that containing the chimeric intron.

After cloning the SV40 and MVM introns, we produced the respective AAV2 vectors comprising the 5′ end portion of the hMYO7A CDS (AAV2-SV40 intron-5′hMYO7A and AAV2-MVM intron-5′hMYO7A) to make a second comparison in vitro in HEK293 cells by MYO7A expression against AAV2-Chimeric intron-5′hMYO7A and AAV2-No intron-5′hMYO7A.

Western blot analysis shows that both SV40 and MVM introns induce comparable expression levels of hMYO7A in vitro (FIG. 3).

Finally, we produced the respective AAV8 vectors comprising the 5′ end portion of the hMYO7A CDS (AAV8-SV40 intron-5′hMYO7A and AAV8-MVM intron-5′hMYO7A). We then performed a third comparison against AAV8-Chimeric intron-5′hMYO7A by Southern Blot of the purified viral DNA, and we also subretinally injected C57BL/6 mice together with AAV8-3′hMYO7A-3×Flag to evaluate hMYO7A expression levels by Western Blot analysis.

We found that both SV40 and MVM introns avoid the formation of the contaminant vector and achieve similar hMYO7A expression levels in vivo (FIG. 4). We decided to use AAV8-SV40 intron-5′hMYO7A for the production of dual AAV8-5′hMYO7A to be used in non-clinical and clinical studies.

Materials and Methods

Generation of AAV Vector Plasmids

The plasmids used for AAV vector production contained the inverted terminal repeats (ITRs) of AAV serotype 2. The two AAV vector plasmids (5′ and 3′) required to generate dual AAV vectors contained several elements. The 5′ plasmid contained: the chicken beta-actin promoter (CBA) and CMV enhancer coupled with the chimeric promoter intron composed of the 5′-donor site from the first intron of the human β-globin gene and the branch and 3′-acceptor site from the intron that is between the leader and the body of an immunoglobulin gene heavy chain variable region (Bothwell et al. (1981) Cell 24: 625-637), a modified version of simian virus 40 promoter's intron (SV40) (Nathwani et al. (2006) Blood 107: 2653-2661) or the minute virus mice intron (Wu et al. (2008) Mol. Ther. 16: 280-289); the N-terminal portion of the transgene coding sequence (CDS); a splice donor sequence. The 3′ plasmid contained: a splice acceptor sequence and the C-terminal portion of the transgene CDS followed by the BGH polyA. For some experiments, a 3′ portion of hMYO7A with the 3×Flag-tag at the C-terminal end was used.

The hMYO7A CDS was split at a natural exon-exon junction, between exons 24-25 (5′ half: NM_000260.3, bp 273-3380; 3′ half: NM_000260.3, bp 3381-6920).

The splice donor (SD) and splice acceptor (SA) sequences contained in dual AAV vector plasmids are as follows:

SD:

(SEQ ID NO: 18)
GTAAGTATCAAGGTTACAAGACAGGTTTAAGGAGACCAATAGAAACTGG
GCTTGTCGAGACAGAGAAGACTCTTGCGTTTCT

SA:

(SEQ ID NO: 19)
GATAGGCACCTATTGGTCTTACTGACATCCACTTTGCCTTTCTCTCCACA
G

The recombinogenic sequence contained in hybrid AK vector plasmids was derived from the phage F1 genome (Gene Bank accession number: J02448.1; bp 5850-5926). The AK sequence is:

(SEQ ID NO: 20)
GGGATTTTTCCGATTTCGGCCTATTGGTTAAAAAATGAGCTGATTTAACA
AAAATTTAACGCGAATTTTAACAAAAT

AAV Vector Production and Characterization

Dual AAV-hMYO7A vectors were produced by the TIGEM AAV Vector Core. Vectors were produced by triple transfection of HEK293 cells followed by two rounds of CsCl₂ purification (Grimm et al. (1998) Hum. Gene Ther. 2760: 2745-2760; Liu et al. (2003) Biotechniques 34: 184-189; Salvetti et al. (1998) Hum. Gene Ther. 9: 695-706; Zolotukhin et al. (1999) Gene Ther. 6: 973-985). For each viral preparation, physical titers [genome copies (GC)/m1] were determined by TaqMan quantitative PCR (Applied Biosystems, Carlsbad, Calif., USA). Primers and probes were designed to anneal on 5′-hMYO7A for AAV-5′hMYO7a and on BGH pA for AAV-3′hMYO7A. The alkaline Southern blot analysis for AAV-5′hMYO7A was carried out as follows: 3E+10 GC of viral DNA were extracted from AAV particles. To digest unpackaged genomes, the vector solution was incubated with 1 U/μL of DNase I (Roche, Milan, Italy) in a total volume of 300 μL containing 40 mM TRIS-HCl, 10 mM NaCl, 6 mM MgCl₂, 1 mM CaCl₂ pH 7.9 for 2 h at 37° C. The DNase I was then inactivated with 50 mM EDTA, followed by incubation with proteinase K and 2.5% N-lauroyl-sarcosil solution at 50° C. for 45 min to lyse the capsids. The DNA was extracted twice with phenol-chloroform and precipitated with two volumes of absolute ethanol and 10% sodium acetate (3 M, pH 7). Purified DNA was run in an alkaline agarose gel and imaged using the Digoxigenin non-radioactive method (Roche, Milan, Italy). 10 μL of the 1 kb DNA ladder (N3232L; New England Biolabs, Ipswich, Mass., USA) were loaded as molecular weight marker. The southern blot probe was obtained by enzymatic digestion of 5′AAV plasmid DNA using KpnI-XhoI to extract and purify a 544 base pair probe.

Cell Culture and Transfection

HEK293 cells were maintained in DMEM supplemented with 10% fetal bovine serum (FBS) (Gibco, Thermo Fisher Scientific, Waltham, Mass., USA). Cells were plated in 6-well plates (HEK293 1E+6 cells/well) and 24 hours later wells were transfected using calcium phosphate+1.5 μg of the corresponding plasmid. After 4 hours, media was replaced with 2 mL of fresh pre-heated media. Cells were harvested and lysed 72 hours post-transfection.

Subretinal Injection of AAV Vectors in Mice

This study was carried out in accordance with the Association for Research in Vision and Ophthalmology Statement for the Use of Animals in Ophthalmic and Vision Research and with the Italian Ministry of Health regulation for animal procedures (authorization no 301/2020-PR).

C57BL/6 and shaker −/− mice were housed at TIGEM animal house (Pozzuoli, Italy) and maintained under a 12 h light/dark cycle (10-50 lux exposure during the light phase). Surgery was performed under anesthesia and all efforts were made to minimise suffering. Adult mice were anesthetised with an intraperitoneal injection of 2 mL/100 g body weight of ketamine/medetomidine. An equal volume of vector solution or excipient were delivered subretinally via a posterior trans-scleral trans-choroidal approach as described in Liang et al. (Liang et al. (2000) Vis. Res. Protoc. 47: 125-139).

Western Blot Analysis

Cells and eyecups (cups+retinas) for Western blot (Wb) analysis were lysed in RIPA buffer (50 mM Tris-HCl pH 8.0, 150 mM NaCl, 1% NP40, 0.5% Na-Deoxycholate, 1 mM EDTA pH 8.0, 0.1% SDS) to extract MYO7A. Lysis buffer were supplemented with 0.5% phenylmethylsulfonyl fluoride (PSMF) (Sigma-Aldrich, St. Louis, Mo., USA) and 1% cOmplete EDTA-free protease inhibitor cocktail (Roche, Milan, Italy). Protein concentration was determined using Pierce BCA protein assay kit (Thermo-Scientific). After lysis, samples were denatured at 99° C. for 5 min in 4× Laemmli sample buffer (Bio-rad, Milan, Italy) supplemented with β-mercaptoethanol 1:10. Samples were separated on 7% acrylamide gels. Antibodies used for immuno-blotting were as follows: anti-3×Flag (1:1000, monoclonal, A8592; Sigma) to recognise full length hMYO7A-3×Flag; anti-Dysferlin (1:500, MONX10795; Tebu-bio, Le Perray-en-Yveline, France). The quantification of Wb bands was performed using ImageJ software, hMYO7A expression was normalised over the expression of Dysferlin.

Vector Sequences

Sequences of MYO7A-encoding vectors used in the experiments are disclosed herein as SEQ ID NOs: 14 and 15.

Sequences of additional vectors used in the experiments are:

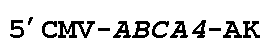

(SEQ ID NO: 21)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG

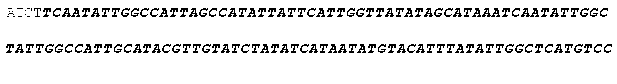

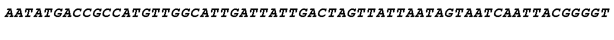

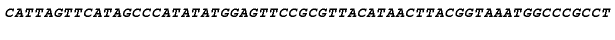

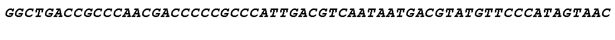

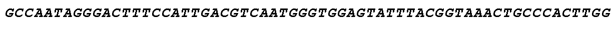

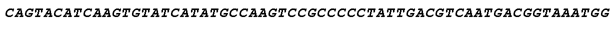

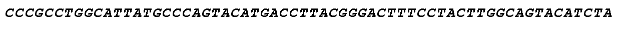

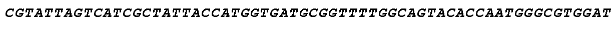

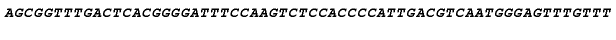

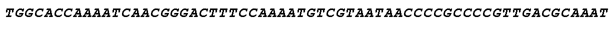

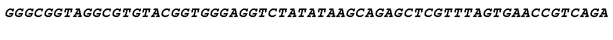

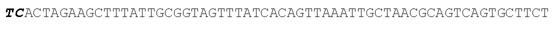

GACACAACAGTCTCGAACTTAAGCTGCAGAAGTTGGTCGTGAGGCACTGGGCAGGTAAGTAT
CAAGGTTACAAGACAGGTTTAAGGAGACCAATAGAAACTGGGCTTGTCGAGACAGAGAAGAC
TCTTGCGTTTCTGATAGGCACCTATTGGTCTTACTGACATCCACTTTGCCTTTCTCTCCACA
GGTGTCCACTCCCAGTTCAATTACAGCTCTTAAGGCTAGAGTACTTAATACGACTCACTATA
GGCTAGCCTCGAGAATTCACGCGTGGTACCTCTAGAGTCGACCCGGGCGGCCGCCATGGGCT
TCGTGAGACAGATACAGCTTTTGCTCTGGAAGAACTGGACCCTGCGGAAAAGGCAAAAGATT
CGCTTTGTGGTGGAACTCGTGTGGCCTTTATCTTTATTTCTGGTCTTGATCTGGTTAAGGAA
TGCCAACCCGCTCTACAGCCATCATGAATGCCATTTCCCCAACAAGGCGATGCCCTCAGCAG
GAATGCTGCCGTGGCTCCAGGGGATCTTCTGCAATGTGAACAATCCCTGTTTTCAAAGCCCC
ACCCCAGGAGAATCTCCTGGAATTGTGTCAAACTATAACAACTCCATCTTGGCAAGGGTATA
TCGAGATTTTCAAGAACTCCTCATGAATGCACCAGAGAGCCAGCACCTTGGCCGTATTTGGA
CAGAGCTACACATCTTGTCCCAATTCATGGACACCCTCCGGACTCACCCGGAGAGAATTGCA
GGAAGAGGAATTCGAATAAGGGATATCTTGAAAGATGAAGAAACACTGACACTATTTCTCAT
TAAAAACATCGGCCTGTCTGACTCAGTGGTCTACCTTCTGATCAACTCTCAAGTCCGTCCAG
AGCAGTTCGCTCATGGAGTCCCGGACCTGGCGCTGAAGGACATCGCCTGCAGCGAGGCCCTC
CTGGAGCGCTTCATCATCTTCAGCCAGAGACGCGGGGCAAAGACGGTGCGCTATGCCCTGTG
CTCCCTCTCCCAGGGCACCCTACAGTGGATAGAAGACACTCTGTATGCCAACGTGGACTTCT
TCAAGCTCTTCCGTGTGCTTCCCACACTCCTAGACAGCCGTTCTCAAGGTATCAATCTGAGA
TCTTGGGGAGGAATATTATCTGATATGTCACCAAGAATTCAAGAGTTTATCCATCGGCCGAG
TATGCAGGACTTGCTGTGGGTGACCAGGCCCCTCATGCAGAATGGTGGTCCAGAGACCTTTA
CAAAGCTGATGGGCATCCTGTCTGACCTCCTGTGTGGCTACCCCGAGGGAGGTGGCTCTCGG
GTGCTCTCCTTCAACTGGTATGAAGACAATAACTATAAGGCCTTTCTGGGGATTGACTCCAC
AAGGAAGGATCCTATCTATTCTTATGACAGAAGAACAACATCCTTTTGTAATGCATTGATCC
AGAGCCTGGAGTCAAATCCTTTAACCAAAATCGCTTGGAGGGCGGCAAAGCCTTTGCTGATG
GGAAAAATCCTGTACACTCCTGATTCACCTGCAGCACGAAGGATACTGAAGAATGCCAACTC
AACTTTTGAAGAACTGGAACACGTTAGGAAGTTGGTCAAAGCCTGGGAAGAAGTAGGGCCCC
AGATCTGGTACTTCTTTGACAACAGCACACAGATGAACATGATCAGAGATACCCTGGGGAAC
CCAACAGTAAAAGACTTTTTGAATAGGCAGCTTGGTGAAGAAGGTATTACTGCTGAAGCCAT
CCTAAACTTCCTCTACAAGGGCCCTCGGGAAAGCCAGGCTGACGACATGGCCAACTTCGACT
GGAGGGACATATTTAACATCACTGATCGCACCCTCCGCCTTGTCAATCAATACCTGGAGTGC
TTGGTCCTGGATAAGTTTGAAAGCTACAATGATGAAACTCAGCTCACCCAACGTGCCCTCTC
TCTACTGGAGGAAAACATGTTCTGGGCCGGAGTGGTATTCCCTGACATGTATCCCTGGACCA
GCTCTCTACCACCCCACGTGAAGTATAAGATCCGAATGGACATAGACGTGGTGGAGAAAACC
AATAAGATTAAAGACAGGTATTGGGATTCTGGTCCCAGAGCTGATCCCGTGGAAGATTTCCG
GTACATCTGGGGCGGGTTTGCCTATCTGCAGGACATGGTTGAACAGGGGATCACAAGGAGCC
AGGTGCAGGCGGAGGCTCCAGTTGGAATCTACCTCCAGCAGATGCCCTACCCCTGCTTCGTG
GACGATTCTTTCATGATCATCCTGAACCGCTGTTTCCCTATCTTCATGGTGCTGGCATGGAT
CTACTCTGTCTCCATGACTGTGAAGAGCATCGTCTTGGAGAAGGAGTTGCGACTGAAGGAGA
CCTTGAAAAATCAGGGTGTCTCCAATGCAGTGATTTGGTGTACCTGGTTCCTGGACAGCTTC
TCCATCATGTCGATGAGCATCTTCCTCCTGACGATATTCATCATGCATGGAAGAATCCTACA
TTACAGCGACCCATTCATCCTCTTCCTGTTCTTGTTGGCTTTCTCCACTGCCACCATCATGC
TGTGCTTTCTGCTCAGCACCTTCTTCTCCAAGGCCAGTCTGGCAGCAGCCTGTAGTGGTGTC
ATCTATTTCACCCTCTACCTGCCACACATCCTGTGCTTCGCCTGGCAGGACCGCATGACCGC
TGAGCTGAAGAAGGCTGTGAGCTTACTGTCTCCGGTGGCATTTGGATTTGGCACTGAGTACC
TGGTTCGCTTTGAAGAGCAAGGCCTGGGGCTGCAGTGGAGCAACATCGGGAACAGTCCCACG
GAAGGGGACGAATTCAGCTTCCTGCTGTCCATGCAGATGATGCTCCTTGATGCTGCTGTCTA
TGGCTTACTCGCTTGGTACCTTGATCAGGTGTTTCCAGGAGACTATGGAACCCCACTTCCTT
GGTACTTTCTTCTACAAGAGTCGTATTGGCTTGGCGGTGAAGGGTGTTCAACCAGAGAAGAA
AGAGCCCTGGAAAAGACCGAGCCCCTAACAGAGGAAACGGAGGATCCAGAGCACCCAGAAGG
AATACACGACTCCTTCTTTGAACGTGAGCATCCAGGGTGGGTTCCTGGGGTATGCGTGAAGA
ATCTGGTAAAGATTTTTGAGCCCTGTGGCCGGCCAGCTGTGGACCGTCTGAACATCACCTTC

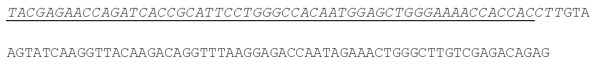

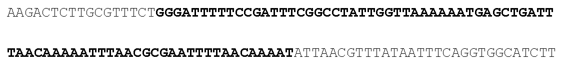

TCCAATTGAGGAACCCCTAGTGATGGAGTTGGCCACTCCCTCTCTGCGCGCTCGCTCGCTCA
CTGAGGCCGGGCGACCAAAGGTCGCCCGACGCCCGGGCTTTGCCCGGGCGGCCTCAGTGAGC
GAGCGAGCGCGCAG
5' ITR

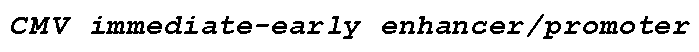

Chimeric intron
5' end portion of ABCA4

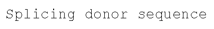

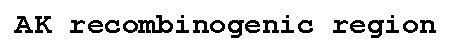

3' ITR

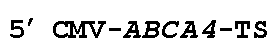

(SEQ ID NO: 22)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG

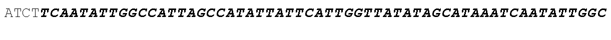

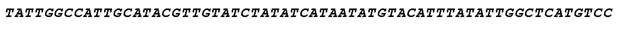

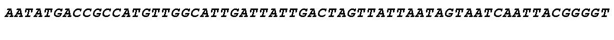

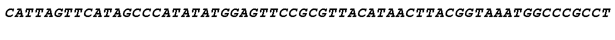

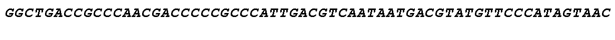

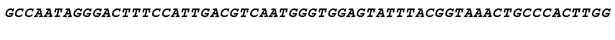

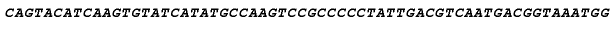

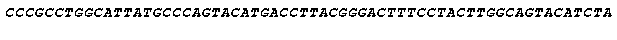

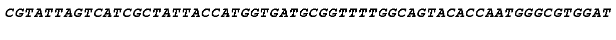

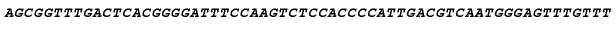

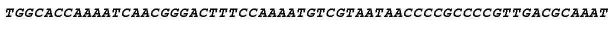

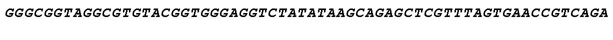

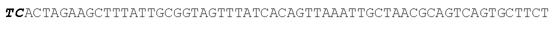

GACACAACAGTCTCGAACTTAAGCTGCAGAAGTTGGTCGTGAGGCACTGGGCAGGTAAGTAT
CAAGGTTACAAGACAGGTTTAAGGAGACCAATAGAAACTGGGCTTGTCGAGACAGAGAAGAC
TCTTGCGTTTCTGATAGGCACCTATTGGTCTTACTGACATCCACTTTGCCTTTCTCTCCACA
GGTGTCCACTCCCAGTTCAATTACAGCTCTTAAGGCTAGAGTACTTAATACGACTCACTATA
GGCTAGCCTCGAGAATTCACGCGTGGTACCTCTAGAGTCGACCCGGGCGGCCGCCATGGGCT
TCGTGAGACAGATACAGCTTTTGCTCTGGAAGAACTGGACCCTGCGGAAAAGGCAAAAGATT
CGCTTTGTGGTGGAACTCGTGTGGCCTTTATCTTTATTTCTGGTCTTGATCTGGTTAAGGAA
TGCCAACCCGCTCTACAGCCATCATGAATGCCATTTCCCCAACAAGGCGATGCCCTCAGCAG
GAATGCTGCCGTGGCTCCAGGGGATCTTCTGCAATGTGAACAATCCCTGTTTTCAAAGCCCC
ACCCCAGGAGAATCTCCTGGAATTGTGTCAAACTATAACAACTCCATCTTGGCAAGGGTATA
TCGAGATTTTCAAGAACTCCTCATGAATGCACCAGAGAGCCAGCACCTTGGCCGTATTTGGA
CAGAGCTACACATCTTGTCCCAATTCATGGACACCCTCCGGACTCACCCGGAGAGAATTGCA
GGAAGAGGAATTCGAATAAGGGATATCTTGAAAGATGAAGAAACACTGACACTATTTCTCAT
TAAAAACATCGGCCTGTCTGACTCAGTGGTCTACCTTCTGATCAACTCTCAAGTCCGTCCAG
AGCAGTTCGCTCATGGAGTCCCGGACCTGGCGCTGAAGGACATCGCCTGCAGCGAGGCCCTC
CTGGAGCGCTTCATCATCTTCAGCCAGAGACGCGGGGCAAAGACGGTGCGCTATGCCCTGTG
CTCCCTCTCCCAGGGCACCCTACAGTGGATAGAAGACACTCTGTATGCCAACGTGGACTTCT
TCAAGCTCTTCCGTGTGCTTCCCACACTCCTAGACAGCCGTTCTCAAGGTATCAATCTGAGA
TCTTGGGGAGGAATATTATCTGATATGTCACCAAGAATTCAAGAGTTTATCCATCGGCCGAG
TATGCAGGACTTGCTGTGGGTGACCAGGCCCCTCATGCAGAATGGTGGTCCAGAGACCTTTA
CAAAGCTGATGGGCATCCTGTCTGACCTCCTGTGTGGCTACCCCGAGGGAGGTGGCTCTCGG
GTGCTCTCCTTCAACTGGTATGAAGACAATAACTATAAGGCCTTTCTGGGGATTGACTCCAC
AAGGAAGGATCCTATCTATTCTTATGACAGAAGAACAACATCCTTTTGTAATGCATTGATCC
AGAGCCTGGAGTCAAATCCTTTAACCAAAATCGCTTGGAGGGCGGCAAAGCCTTTGCTGATG
GGAAAAATCCTGTACACTCCTGATTCACCTGCAGCACGAAGGATACTGAAGAATGCCAACTC
AACTTTTGAAGAACTGGAACACGTTAGGAAGTTGGTCAAAGCCTGGGAAGAAGTAGGGCCCC
AGATCTGGTACTTCTTTGACAACAGCACACAGATGAACATGATCAGAGATACCCTGGGGAAC
CCAACAGTAAAAGACTTTTTGAATAGGCAGCTTGGTGAAGAAGGTATTACTGCTGAAGCCAT
CCTAAACTTCCTCTACAAGGGCCCTCGGGAAAGCCAGGCTGACGACATGGCCAACTTCGACT
GGAGGGACATATTTAACATCACTGATCGCACCCTCCGCCTTGTCAATCAATACCTGGAGTGC
TTGGTCCTGGATAAGTTTGAAAGCTACAATGATGAAACTCAGCTCACCCAACGTGCCCTCTC
TCTACTGGAGGAAAACATGTTCTGGGCCGGAGTGGTATTCCCTGACATGTATCCCTGGACCA
GCTCTCTACCACCCCACGTGAAGTATAAGATCCGAATGGACATAGACGTGGTGGAGAAAACC
AATAAGATTAAAGACAGGTATTGGGATTCTGGTCCCAGAGCTGATCCCGTGGAAGATTTCCG
GTACATCTGGGGCGGGTTTGCCTATCTGCAGGACATGGTTGAACAGGGGATCACAAGGAGCC
AGGTGCAGGCGGAGGCTCCAGTTGGAATCTACCTCCAGCAGATGCCCTACCCCTGCTTCGTG
GACGATTCTTTCATGATCATCCTGAACCGCTGTTTCCCTATCTTCATGGTGCTGGCATGGAT
CTACTCTGTCTCCATGACTGTGAAGAGCATCGTCTTGGAGAAGGAGTTGCGACTGAAGGAGA
CCTTGAAAAATCAGGGTGTCTCCAATGCAGTGATTTGGTGTACCTGGTTCCTGGACAGCTTC
TCCATCATGTCGATGAGCATCTTCCTCCTGACGATATTCATCATGCATGGAAGAATCCTACA
TTACAGCGACCCATTCATCCTCTTCCTGTTCTTGTTGGCTTTCTCCACTGCCACCATCATGC
TGTGCTTTCTGCTCAGCACCTTCTTCTCCAAGGCCAGTCTGGCAGCAGCCTGTAGTGGTGTC
ATCTATTTCACCCTCTACCTGCCACACATCCTGTGCTTCGCCTGGCAGGACCGCATGACCGC
TGAGCTGAAGAAGGCTGTGAGCTTACTGTCTCCGGTGGCATTTGGATTTGGCACTGAGTACC
TGGTTCGCTTTGAAGAGCAAGGCCTGGGGCTGCAGTGGAGCAACATCGGGAACAGTCCCACG
GAAGGGGACGAATTCAGCTTCCTGCTGTCCATGCAGATGATGCTCCTTGATGCTGCTGTCTA
TGGCTTACTCGCTTGGTACCTTGATCAGGTGTTTCCAGGAGACTATGGAACCCCACTTCCTT
GGTACTTTCTTCTACAAGAGTCGTATTGGCTTGGCGGTGAAGGGTGTTCAACCAGAGAAGAA
AGAGCCCTGGAAAAGACCGAGCCCCTAACAGAGGAAACGGAGGATCCAGAGCACCCAGAAGG
AATACACGACTCCTTCTTTGAACGTGAGCATCCAGGGTGGGTTCCTGGGGTATGCGTGAAGA
ATCTGGTAAAGATTTTTGAGCCCTGTGGCCGGCCAGCTGTGGACCGTCTGAACATCACCTTC

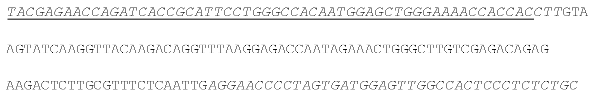

GCGCTCGCTCGCTCACTGAGGCCGGGCGACCAAAGGTCGCCCGACGCCCGGGCTTTGCCCGG
GCGGCCTCAGTGAGCGAGCGAGCGCGCAG
5′ ITR

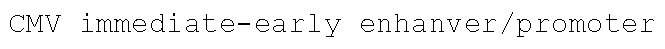

Chimeric intron
5' end portion of ABCA4

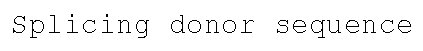

3' ITR

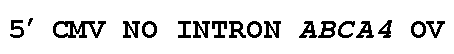

(SEQ ID NO: 23)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG

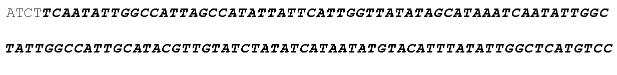

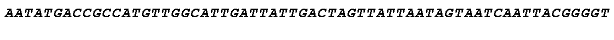

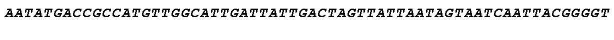

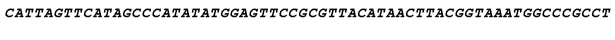

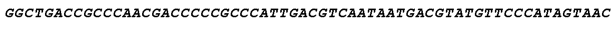

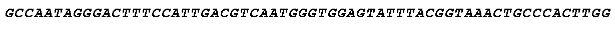

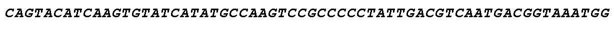

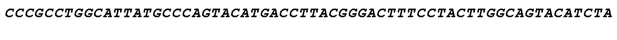

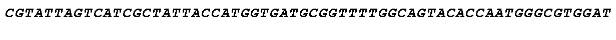

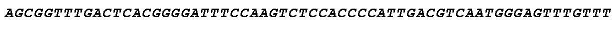

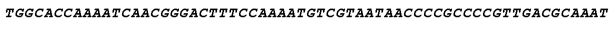

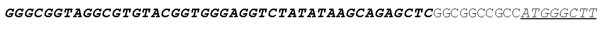

CGTGAGACAGATACAGCTTTTGCTCTGGAAGAACTGGACCCTGCGGAAAAGGCAAAAGATTC
GCTTTGTGGTGGAACTCGTGTGGCCTTTATCTTTATTTCTGGTCTTGATCTGGTTAAGGAAT
GCCAACCCGCTCTACAGCCATCATGAATGCCATTTCCCCAACAAGGCGATGCCCTCAGCAGG
AATGCTGCCGTGGCTCCAGGGGATCTTCTGCAATGTGAACAATCCCTGTTTTCAAAGCCCCA
CCCCAGGAGAATCTCCTGGAATTGTGTCAAACTATAACAACTCCATCTTGGCAAGGGTATAT
CGAGATTTTCAAGAACTCCTCATGAATGCACCAGAGAGCCAGCACCTTGGCCGTATTTGGAC
AGAGCTACACATCTTGTCCCAATTCATGGACACCCTCCGGACTCACCCGGAGAGAATTGCAG
GAAGAGGAATTCGAATAAGGGATATCTTGAAAGATGAAGAAACACTGACACTATTTCTCATT
AAAAACATCGGCCTGTCTGACTCAGTGGTCTACCTTCTGATCAACTCTCAAGTCCGTCCAGA
GCAGTTCGCTCATGGAGTCCCGGACCTGGCGCTGAAGGACATCGCCTGCAGCGAGGCCCTCC
TGGAGCGCTTCATCATCTTCAGCCAGAGACGCGGGGCAAAGACGGTGCGCTATGCCCTGTGC
TCCCTCTCCCAGGGCACCCTACAGTGGATAGAAGACACTCTGTATGCCAACGTGGACTTCTT
CAAGCTCTTCCGTGTGCTTCCCACACTCCTAGACAGCCGTTCTCAAGGTATCAATCTGAGAT
CTTGGGGAGGAATATTATCTGATATGTCACCAAGAATTCAAGAGTTTATCCATCGGCCGAGT
ATGCAGGACTTGCTGTGGGTGACCAGGCCCCTCATGCAGAATGGTGGTCCAGAGACCTTTAC
AAAGCTGATGGGCATCCTGTCTGACCTCCTGTGTGGCTACCCCGAGGGAGGTGGCTCTCGGG
TGCTCTCCTTCAACTGGTATGAAGACAATAACTATAAGGCCTTTCTGGGGATTGACTCCACA
AGGAAGGATCCTATCTATTCTTATGACAGAAGAACAACATCCTTTTGTAATGCATTGATCCA
GAGCCTGGAGTCAAATCCTTTAACCAAAATCGCTTGGAGGGCGGCAAAGCCTTTGCTGATGG
GAAAAATCCTGTACACTCCTGATTCACCTGCAGCACGAAGGATACTGAAGAATGCCAACTCA
ACTTTTGAAGAACTGGAACACGTTAGGAAGTTGGTCAAAGCCTGGGAAGAAGTAGGGCCCCA
GATCTGGTACTTCTTTGACAACAGCACACAGATGAACATGATCAGAGATACCCTGGGGAACC
CAACAGTAAAAGACTTTTTGAATAGGCAGCTTGGTGAAGAAGGTATTACTGCTGAAGCCATC
CTAAACTTCCTCTACAAGGGCCCTCGGGAAAGCCAGGCTGACGACATGGCCAACTTCGACTG
GAGGGACATATTTAACATCACTGATCGCACCCTCCGCCTTGTCAATCAATACCTGGAGTGCT
TGGTCCTGGATAAGTTTGAAAGCTACAATGATGAAACTCAGCTCACCCAACGTGCCCTCTCT
CTACTGGAGGAAAACATGTTCTGGGCCGGAGTGGTATTCCCTGACATGTATCCCTGGACCAG
CTCTCTACCACCCCACGTGAAGTATAAGATCCGAATGGACATAGACGTGGTGGAGAAAACCA
ATAAGATTAAAGACAGGTATTGGGATTCTGGTCCCAGAGCTGATCCCGTGGAAGATTTCCGG
TACATCTGGGGCGGGTTTGCCTATCTGCAGGACATGGTTGAACAGGGGATCACAAGGAGCCA
GGTGCAGGCGGAGGCTCCAGTTGGAATCTACCTCCAGCAGATGCCCTACCCCTGCTTCGTGG
ACGATTCTTTCATGATCATCCTGAACCGCTGTTTCCCTATCTTCATGGTGCTGGCATGGATC
TACTCTGTCTCCATGACTGTGAAGAGCATCGTCTTGGAGAAGGAGTTGCGACTGAAGGAGAC
CTTGAAAAATCAGGGTGTCTCCAATGCAGTGATTTGGTGTACCTGGTTCCTGGACAGCTTCT
CCATCATGTCGATGAGCATCTTCCTCCTGACGATATTCATCATGCATGGAAGAATCCTACAT
TACAGCGACCCATTCATCCTCTTCCTGTTCTTGTTGGCTTTCTCCACTGCCACCATCATGCT
GTGCTTTCTGCTCAGCACCTTCTTCTCCAAGGCCAGTCTGGCAGCAGCCTGTAGTGGTGTCA
TCTATTTCACCCTCTACCTGCCACACATCCTGTGCTTCGCCTGGCAGGACCGCATGACCGCT
GAGCTGAAGAAGGCTGTGAGCTTACTGTCTCCGGTGGCATTTGGATTTGGCACTGAGTACCT
GGTTCGCTTTGAAGAGCAAGGCCTGGGGCTGCAGTGGAGCAACATCGGGAACAGTCCCACGG
AAGGGGACGAATTCAGCTTCCTGCTGTCCATGCAGATGATGCTCCTTGATGCTGCTGTCTAT
GGCTTACTCGCTTGGTACCTTGATCAGGTGTTTCCAGGAGACTATGGAACCCCACTTCCTTG
GTACTTTCTTCTACAAGAGTCGTATTGGCTTGGCGGTGAAGGGTGTTCAACCAGAGAAGAAA
GAGCCCTGGAAAAGACCGAGCCCCTAACAGAGGAAACGGAGGATCCAGAGCACCCAGAAGGA
ATACACGACTCCTTCTTTGAACGTGAGCATCCAGGGTGGGTTCCTGGGGTATGCGTGAAGAA
TCTGGTAAAGATTTTTGAGCCCTGTGGCCGGCCAGCTGTGGACCGTCTGAACATCACCTTCT
ACGAGAACCAGATCACCGCATTCCTGGGCCACAATGGAGCTGGGAAAACCACCACCTTGTCC
ATCCTGACGGGTCTGTTGCCACCAACCTCTGGGACTGTGCTCGTTGGGGGAAGGGACATTGA
AACCAGCCTGGATGCAGTCCGGCAGAGCCTTGGCATGTGTCCACAGCACAACATCCTGTTCC
ACCACCTCACGGTGGCTGAGCACATGCTGTTCTATGCCCAGCTGAAAGGAAAGTCCCAGGAG
GAGGCCCAGCTGGAGATGGAAGCCATGTTGGAGGACACAGGCCTCCACCACAAGCGGAATGA
AGAGGCTCAGGACCTATCAGGTGGCATGCAGAGAAAGCTGTCGGTTGCCATTGCCTTTGTGG
GAGATGCCAAGGTGGTGATTCTGGACGAACCCACCTCTGGGGTGGACCCTTACTCGAGACGC
TCAATCTGGGATCTGCTCCTGAAGTATCGCTCAGGCAGAACCATCATCATGTCCACTCACCA
CATGGACGAGGCCGACCTCCTTGGGGACCGCATTGCCATCATTGCCCAGGGAAGGCTCTACT
GCTCAGGCACCCCACTCTTCCTGAAGAACTGCTTTGGCACAGGCTTGTACTTAACCTTGGTG
CGCAAGGAACCCCTAGTGATGGAGTTGGCCACTCCCTCTCTGCGCGCTCGCTCGCTCACTGA
GGCCGGGCGACCAAAGGTCGCCCGACGCCCGGGCTTTGCCCGGGCGGCCTCAGTGAGCGAGC
GAGCGCGCAG
5' ITR

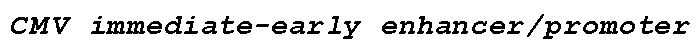

5' end portion of ABCA4
3' ITR

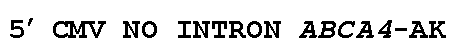

(SEQ ID NO: 24)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG

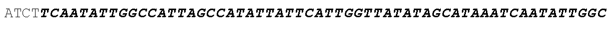

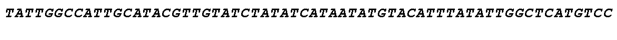

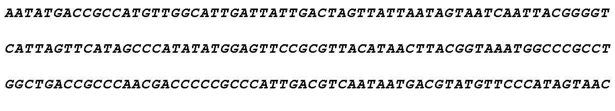

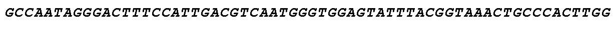

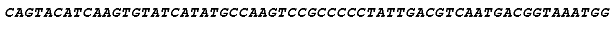

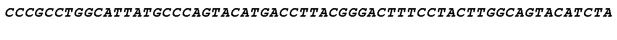

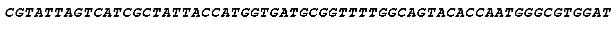

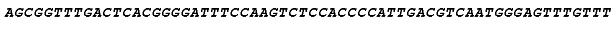

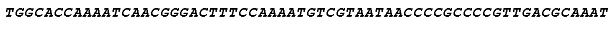

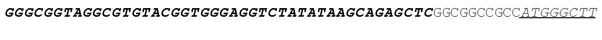

CGTGAGACAGATACAGCTTTTGCTCTGGAAGAACTGGACCCTGCGGAAAAGGCAAAAGATTC
GCTTTGTGGTGGAACTCGTGTGGCCTTTATCTTTATTTCTGGTCTTGATCTGGTTAAGGAAT
GCCAACCCGCTCTACAGCCATCATGAATGCCATTTCCCCAACAAGGCGATGCCCTCAGCAGG
AATGCTGCCGTGGCTCCAGGGGATCTTCTGCAATGTGAACAATCCCTGTTTTCAAAGCCCCA
CCCCAGGAGAATCTCCTGGAATTGTGTCAAACTATAACAACTCCATCTTGGCAAGGGTATAT
CGAGATTTTCAAGAACTCCTCATGAATGCACCAGAGAGCCAGCACCTTGGCCGTATTTGGAC
AGAGCTACACATCTTGTCCCAATTCATGGACACCCTCCGGACTCACCCGGAGAGAATTGCAG
GAAGAGGAATTCGAATAAGGGATATCTTGAAAGATGAAGAAACACTGACACTATTTCTCATT
AAAAACATCGGCCTGTCTGACTCAGTGGTCTACCTTCTGATCAACTCTCAAGTCCGTCCAGA
GCAGTTCGCTCATGGAGTCCCGGACCTGGCGCTGAAGGACATCGCCTGCAGCGAGGCCCTCC
TGGAGCGCTTCATCATCTTCAGCCAGAGACGCGGGGCAAAGACGGTGCGCTATGCCCTGTGC
TCCCTCTCCCAGGGCACCCTACAGTGGATAGAAGACACTCTGTATGCCAACGTGGACTTCTT
CAAGCTCTTCCGTGTGCTTCCCACACTCCTAGACAGCCGTTCTCAAGGTATCAATCTGAGAT
CTTGGGGAGGAATATTATCTGATATGTCACCAAGAATTCAAGAGTTTATCCATCGGCCGAGT
ATGCAGGACTTGCTGTGGGTGACCAGGCCCCTCATGCAGAATGGTGGTCCAGAGACCTTTAC
AAAGCTGATGGGCATCCTGTCTGACCTCCTGTGTGGCTACCCCGAGGGAGGTGGCTCTCGGG
TGCTCTCCTTCAACTGGTATGAAGACAATAACTATAAGGCCTTTCTGGGGATTGACTCCACA
AGGAAGGATCCTATCTATTCTTATGACAGAAGAACAACATCCTTTTGTAATGCATTGATCCA
GAGCCTGGAGTCAAATCCTTTAACCAAAATCGCTTGGAGGGCGGCAAAGCCTTTGCTGATGG
GAAAAATCCTGTACACTCCTGATTCACCTGCAGCACGAAGGATACTGAAGAATGCCAACTCA
ACTTTTGAAGAACTGGAACACGTTAGGAAGTTGGTCAAAGCCTGGGAAGAAGTAGGGCCCCA
GATCTGGTACTTCTTTGACAACAGCACACAGATGAACATGATCAGAGATACCCTGGGGAACC
CAACAGTAAAAGACTTTTTGAATAGGCAGCTTGGTGAAGAAGGTATTACTGCTGAAGCCATC
CTAAACTTCCTCTACAAGGGCCCTCGGGAAAGCCAGGCTGACGACATGGCCAACTTCGACTG
GAGGGACATATTTAACATCACTGATCGCACCCTCCGCCTTGTCAATCAATACCTGGAGTGCT
TGGTCCTGGATAAGTTTGAAAGCTACAATGATGAAACTCAGCTCACCCAACGTGCCCTCTCT
CTACTGGAGGAAAACATGTTCTGGGCCGGAGTGGTATTCCCTGACATGTATCCCTGGACCAG
CTCTCTACCACCCCACGTGAAGTATAAGATCCGAATGGACATAGACGTGGTGGAGAAAACCA
ATAAGATTAAAGACAGGTATTGGGATTCTGGTCCCAGAGCTGATCCCGTGGAAGATTTCCGG
TACATCTGGGGCGGGTTTGCCTATCTGCAGGACATGGTTGAACAGGGGATCACAAGGAGCCA
GGTGCAGGCGGAGGCTCCAGTTGGAATCTACCTCCAGCAGATGCCCTACCCCTGCTTCGTGG
ACGATTCTTTCATGATCATCCTGAACCGCTGTTTCCCTATCTTCATGGTGCTGGCATGGATC
TACTCTGTCTCCATGACTGTGAAGAGCATCGTCTTGGAGAAGGAGTTGCGACTGAAGGAGAC
CTTGAAAAATCAGGGTGTCTCCAATGCAGTGATTTGGTGTACCTGGTTCCTGGACAGCTTCT
CCATCATGTCGATGAGCATCTTCCTCCTGACGATATTCATCATGCATGGAAGAATCCTACAT
TACAGCGACCCATTCATCCTCTTCCTGTTCTTGTTGGCTTTCTCCACTGCCACCATCATGCT
GTGCTTTCTGCTCAGCACCTTCTTCTCCAAGGCCAGTCTGGCAGCAGCCTGTAGTGGTGTCA
TCTATTTCACCCTCTACCTGCCACACATCCTGTGCTTCGCCTGGCAGGACCGCATGACCGCT
GAGCTGAAGAAGGCTGTGAGCTTACTGTCTCCGGTGGCATTTGGATTTGGCACTGAGTACCT
GGTTCGCTTTGAAGAGCAAGGCCTGGGGCTGCAGTGGAGCAACATCGGGAACAGTCCCACGG
AAGGGGACGAATTCAGCTTCCTGCTGTCCATGCAGATGATGCTCCTTGATGCTGCTGTCTAT
GGCTTACTCGCTTGGTACCTTGATCAGGTGTTTCCAGGAGACTATGGAACCCCACTTCCTTG
GTACTTTCTTCTACAAGAGTCGTATTGGCTTGGCGGTGAAGGGTGTTCAACCAGAGAAGAAA
GAGCCCTGGAAAAGACCGAGCCCCTAACAGAGGAAACGGAGGATCCAGAGCACCCAGAAGGA
ATACACGACTCCTTCTTTGAACGTGAGCATCCAGGGTGGGTTCCTGGGGTATGCGTGAAGAA
TCTGGTAAAGATTTTTGAGCCCTGTGGCCGGCCAGCTGTGGACCGTCTGAACATCACCTTCT

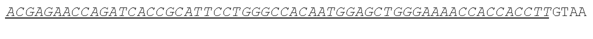

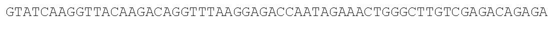

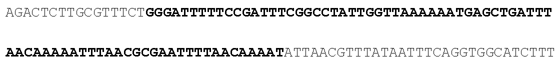

CCAATTGAGGAACCCCTAGTGATGGAGTTGGCCACTCCCTCTCTGCGCGCTCGCTCGCTCAC
TGAGGCCGGGCGACCAAAGGTCGCCCGACGCCCGGGCTTTGCCCGGGCGGCCTCAGTGAGCG
AGCGAGCGCGCAG
5' ITR

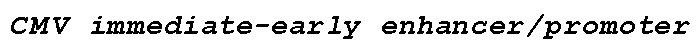

5' end portion of ABCA4

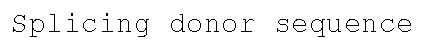

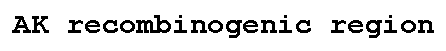

3' ITR

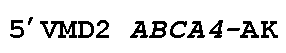

(SEQ ID NO: 25)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG

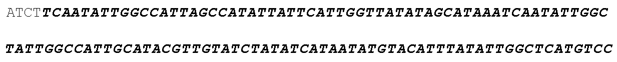

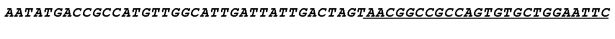

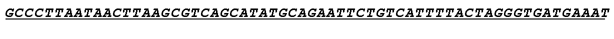

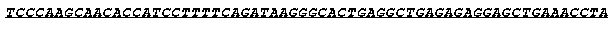

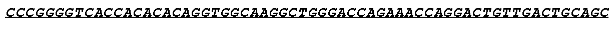

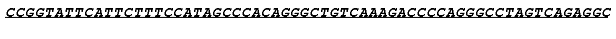

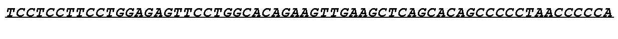

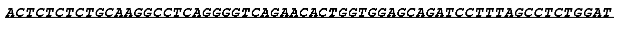

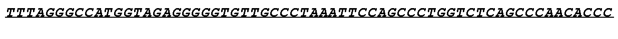

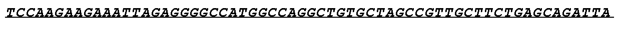

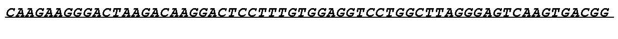

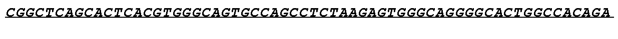

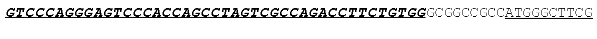

TGAGACAGATACAGCTTTTGCTCTGGAAGAACTGGACCCTGCGGAAAAGGCAAAAGATTCGC
TTTGTGGTGGAACTCGTGTGGCCTTTATCTTTATTTCTGGTCTTGATCTGGTTAAGGAATGC
CAACCCGCTCTACAGCCATCATGAATGCCATTTCCCCAACAAGGCGATGCCCTCAGCAGGAA
TGCTGCCGTGGCTCCAGGGGATCTTCTGCAATGTGAACAATCCCTGTTTTCAAAGCCCCACC
CCAGGAGAATCTCCTGGAATTGTGTCAAACTATAACAACTCCATCTTGGCAAGGGTATATCG
AGATTTTCAAGAACTCCTCATGAATGCACCAGAGAGCCAGCACCTTGGCCGTATTTGGACAG
AGCTACACATCTTGTCCCAATTCATGGACACCCTCCGGACTCACCCGGAGAGAATTGCAGGA
AGAGGAATTCGAATAAGGGATATCTTGAAAGATGAAGAAACACTGACACTATTTCTCATTAA
AAACATCGGCCTGTCTGACTCAGTGGTCTACCTTCTGATCAACTCTCAAGTCCGTCCAGAGC
AGTTCGCTCATGGAGTCCCGGACCTGGCGCTGAAGGACATCGCCTGCAGCGAGGCCCTCCTG
GAGCGCTTCATCATCTTCAGCCAGAGACGCGGGGCAAAGACGGTGCGCTATGCCCTGTGCTC
CCTCTCCCAGGGCACCCTACAGTGGATAGAAGACACTCTGTATGCCAACGTGGACTTCTTCA
AGCTCTTCCGTGTGCTTCCCACACTCCTAGACAGCCGTTCTCAAGGTATCAATCTGAGATCT
TGGGGAGGAATATTATCTGATATGTCACCAAGAATTCAAGAGTTTATCCATCGGCCGAGTAT
GCAGGACTTGCTGTGGGTGACCAGGCCCCTCATGCAGAATGGTGGTCCAGAGACCTTTACAA
AGCTGATGGGCATCCTGTCTGACCTCCTGTGTGGCTACCCCGAGGGAGGTGGCTCTCGGGTG
CTCTCCTTCAACTGGTATGAAGACAATAACTATAAGGCCTTTCTGGGGATTGACTCCACAAG
GAAGGATCCTATCTATTCTTATGACAGAAGAACAACATCCTTTTGTAATGCATTGATCCAGA
GCCTGGAGTCAAATCCTTTAACCAAAATCGCTTGGAGGGCGGCAAAGCCTTTGCTGATGGGA
AAAATCCTGTACACTCCTGATTCACCTGCAGCACGAAGGATACTGAAGAATGCCAACTCAAC
TTTTGAAGAACTGGAACACGTTAGGAAGTTGGTCAAAGCCTGGGAAGAAGTAGGGCCCCAGA
TCTGGTACTTCTTTGACAACAGCACACAGATGAACATGATCAGAGATACCCTGGGGAACCCA
ACAGTAAAAGACTTTTTGAATAGGCAGCTTGGTGAAGAAGGTATTACTGCTGAAGCCATCCT
AAACTTCCTCTACAAGGGCCCTCGGGAAAGCCAGGCTGACGACATGGCCAACTTCGACTGGA
GGGACATATTTAACATCACTGATCGCACCCTCCGCCTTGTCAATCAATACCTGGAGTGCTTG
GTCCTGGATAAGTTTGAAAGCTACAATGATGAAACTCAGCTCACCCAACGTGCCCTCTCTCT
ACTGGAGGAAAACATGTTCTGGGCCGGAGTGGTATTCCCTGACATGTATCCCTGGACCAGCT
CTCTACCACCCCACGTGAAGTATAAGATCCGAATGGACATAGACGTGGTGGAGAAAACCAAT
AAGATTAAAGACAGGTATTGGGATTCTGGTCCCAGAGCTGATCCCGTGGAAGATTTCCGGTA
CATCTGGGGCGGGTTTGCCTATCTGCAGGACATGGTTGAACAGGGGATCACAAGGAGCCAGG
TGCAGGCGGAGGCTCCAGTTGGAATCTACCTCCAGCAGATGCCCTACCCCTGCTTCGTGGAC
GATTCTTTCATGATCATCCTGAACCGCTGTTTCCCTATCTTCATGGTGCTGGCATGGATCTA
CTCTGTCTCCATGACTGTGAAGAGCATCGTCTTGGAGAAGGAGTTGCGACTGAAGGAGACCT
TGAAAAATCAGGGTGTCTCCAATGCAGTGATTTGGTGTACCTGGTTCCTGGACAGCTTCTCC
ATCATGTCGATGAGCATCTTCCTCCTGACGATATTCATCATGCATGGAAGAATCCTACATTA
CAGCGACCCATTCATCCTCTTCCTGTTCTTGTTGGCTTTCTCCACTGCCACCATCATGCTGT
GCTTTCTGCTCAGCACCTTCTTCTCCAAGGCCAGTCTGGCAGCAGCCTGTAGTGGTGTCATC
TATTTCACCCTCTACCTGCCACACATCCTGTGCTTCGCCTGGCAGGACCGCATGACCGCTGA
GCTGAAGAAGGCTGTGAGCTTACTGTCTCCGGTGGCATTTGGATTTGGCACTGAGTACCTGG
TTCGCTTTGAAGAGCAAGGCCTGGGGCTGCAGTGGAGCAACATCGGGAACAGTCCCACGGAA
GGGGACGAATTCAGCTTCCTGCTGTCCATGCAGATGATGCTCCTTGATGCTGCTGTCTATGG
CTTACTCGCTTGGTACCTTGATCAGGTGTTTCCAGGAGACTATGGAACCCCACTTCCTTGGT
ACTTTCTTCTACAAGAGTCGTATTGGCTTGGCGGTGAAGGGTGTTCAACCAGAGAAGAAAGA
GCCCTGGAAAAGACCGAGCCCCTAACAGAGGAAACGGAGGATCCAGAGCACCCAGAAGGAAT
ACACGACTCCTTCTTTGAACGTGAGCATCCAGGGTGGGTTCCTGGGGTATGCGTGAAGAATC
TGGTAAAGATTTTTGAGCCCTGTGGCCGGCCAGCTGTGGACCGTCTGAACATCACCTTCTAC

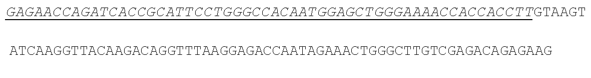

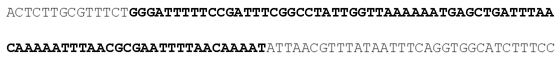

AATTGAGGAACCCCTAGTGATGGAGTTGGCCACTCCCTCTCTGCGCGCTCGCTCGCTCACTG
AGGCCGGGCGACCAAAGGTCGCCCGACGCCCGGGCTTTGCCCGGGCGGCCTCAGTGAGCGAG
CGAGCGCGCAG
5' ITR

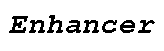

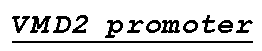

5' end portion of ABCA4

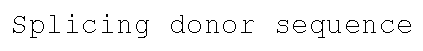

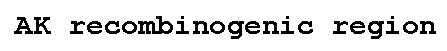

3' ITR

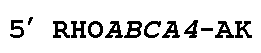

(SEQ ID NO: 26)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG

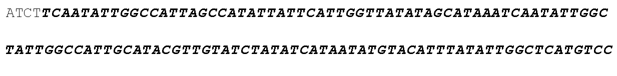

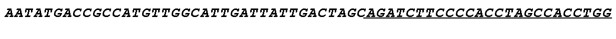

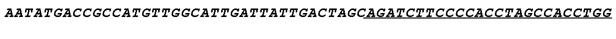

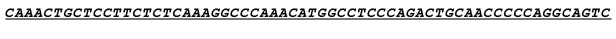

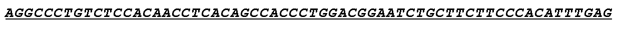

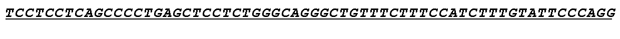

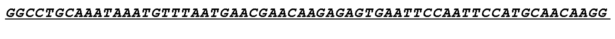

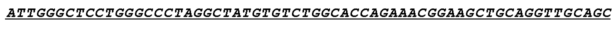

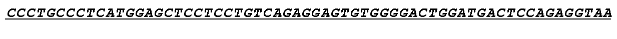

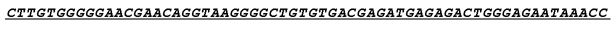

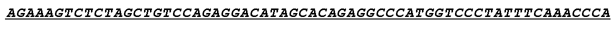

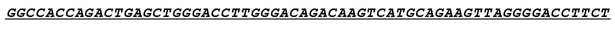

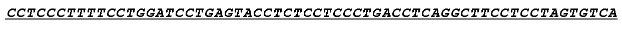

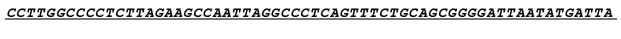

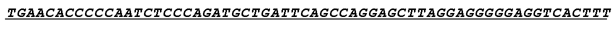

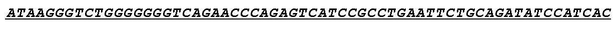

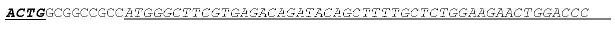

TGCGGAAAAGGCAAAAGATTCGCTTTGTGGTGGAACTCGTGTGGCCTTTATCTTTATTTCTG
GTCTTGATCTGGTTAAGGAATGCCAACCCGCTCTACAGCCATCATGAATGCCATTTCCCCAA
CAAGGCGATGCCCTCAGCAGGAATGCTGCCGTGGCTCCAGGGGATCTTCTGCAATGTGAACA
ATCCCTGTTTTCAAAGCCCCACCCCAGGAGAATCTCCTGGAATTGTGTCAAACTATAACAAC
TCCATCTTGGCAAGGGTATATCGAGATTTTCAAGAACTCCTCATGAATGCACCAGAGAGCCA
GCACCTTGGCCGTATTTGGACAGAGCTACACATCTTGTCCCAATTCATGGACACCCTCCGGA
CTCACCCGGAGAGAATTGCAGGAAGAGGAATTCGAATAAGGGATATCTTGAAAGATGAAGAA
ACACTGACACTATTTCTCATTAAAAACATCGGCCTGTCTGACTCAGTGGTCTACCTTCTGAT
CAACTCTCAAGTCCGTCCAGAGCAGTTCGCTCATGGAGTCCCGGACCTGGCGCTGAAGGACA
TCGCCTGCAGCGAGGCCCTCCTGGAGCGCTTCATCATCTTCAGCCAGAGACGCGGGGCAAAG
ACGGTGCGCTATGCCCTGTGCTCCCTCTCCCAGGGCACCCTACAGTGGATAGAAGACACTCT
GTATGCCAACGTGGACTTCTTCAAGCTCTTCCGTGTGCTTCCCACACTCCTAGACAGCCGTT
CTCAAGGTATCAATCTGAGATCTTGGGGAGGAATATTATCTGATATGTCACCAAGAATTCAA
GAGTTTATCCATCGGCCGAGTATGCAGGACTTGCTGTGGGTGACCAGGCCCCTCATGCAGAA
TGGTGGTCCAGAGACCTTTACAAAGCTGATGGGCATCCTGTCTGACCTCCTGTGTGGCTACC
CCGAGGGAGGTGGCTCTCGGGTGCTCTCCTTCAACTGGTATGAAGACAATAACTATAAGGCC
TTTCTGGGGATTGACTCCACAAGGAAGGATCCTATCTATTCTTATGACAGAAGAACAACATC
CTTTTGTAATGCATTGATCCAGAGCCTGGAGTCAAATCCTTTAACCAAAATCGCTTGGAGGG
CGGCAAAGCCTTTGCTGATGGGAAAAATCCTGTACACTCCTGATTCACCTGCAGCACGAAGG
ATACTGAAGAATGCCAACTCAACTTTTGAAGAACTGGAACACGTTAGGAAGTTGGTCAAAGC
CTGGGAAGAAGTAGGGCCCCAGATCTGGTACTTCTTTGACAACAGCACACAGATGAACATGA
TCAGAGATACCCTGGGGAACCCAACAGTAAAAGACTTTTTGAATAGGCAGCTTGGTGAAGAA
GGTATTACTGCTGAAGCCATCCTAAACTTCCTCTACAAGGGCCCTCGGGAAAGCCAGGCTGA
CGACATGGCCAACTTCGACTGGAGGGACATATTTAACATCACTGATCGCACCCTCCGCCTTG
TCAATCAATACCTGGAGTGCTTGGTCCTGGATAAGTTTGAAAGCTACAATGATGAAACTCAG
CTCACCCAACGTGCCCTCTCTCTACTGGAGGAAAACATGTTCTGGGCCGGAGTGGTATTCCC
TGACATGTATCCCTGGACCAGCTCTCTACCACCCCACGTGAAGTATAAGATCCGAATGGACA
TAGACGTGGTGGAGAAAACCAATAAGATTAAAGACAGGTATTGGGATTCTGGTCCCAGAGCT
GATCCCGTGGAAGATTTCCGGTACATCTGGGGCGGGTTTGCCTATCTGCAGGACATGGTTGA
ACAGGGGATCACAAGGAGCCAGGTGCAGGCGGAGGCTCCAGTTGGAATCTACCTCCAGCAGA
TGCCCTACCCCTGCTTCGTGGACGATTCTTTCATGATCATCCTGAACCGCTGTTTCCCTATC
TTCATGGTGCTGGCATGGATCTACTCTGTCTCCATGACTGTGAAGAGCATCGTCTTGGAGAA
GGAGTTGCGACTGAAGGAGACCTTGAAAAATCAGGGTGTCTCCAATGCAGTGATTTGGTGTA
CCTGGTTCCTGGACAGCTTCTCCATCATGTCGATGAGCATCTTCCTCCTGACGATATTCATC
ATGCATGGAAGAATCCTACATTACAGCGACCCATTCATCCTCTTCCTGTTCTTGTTGGCTTT
CTCCACTGCCACCATCATGCTGTGCTTTCTGCTCAGCACCTTCTTCTCCAAGGCCAGTCTGG
CAGCAGCCTGTAGTGGTGTCATCTATTTCACCCTCTACCTGCCACACATCCTGTGCTTCGCC
TGGCAGGACCGCATGACCGCTGAGCTGAAGAAGGCTGTGAGCTTACTGTCTCCGGTGGCATT
TGGATTTGGCACTGAGTACCTGGTTCGCTTTGAAGAGCAAGGCCTGGGGCTGCAGTGGAGCA
ACATCGGGAACAGTCCCACGGAAGGGGACGAATTCAGCTTCCTGCTGTCCATGCAGATGATG
CTCCTTGATGCTGCTGTCTATGGCTTACTCGCTTGGTACCTTGATCAGGTGTTTCCAGGAGA
CTATGGAACCCCACTTCCTTGGTACTTTCTTCTACAAGAGTCGTATTGGCTTGGCGGTGAAG
GGTGTTCAACCAGAGAAGAAAGAGCCCTGGAAAAGACCGAGCCCCTAACAGAGGAAACGGAG
GATCCAGAGCACCCAGAAGGAATACACGACTCCTTCTTTGAACGTGAGCATCCAGGGTGGGT
TCCTGGGGTATGCGTGAAGAATCTGGTAAAGATTTTTGAGCCCTGTGGCCGGCCAGCTGTGG
ACCGTCTGAACATCACCTTCTACGAGAACCAGATCACCGCATTCCTGGGCCACAATGGAGCT

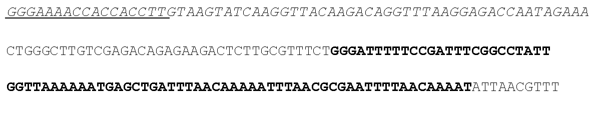

ATAATTTCAGGTGGCATCTTTCCAATTGAGGAACCCCTAGTGATGGAGTTGGCCACTCCCTC
TCTGCGCGCTCGCTCGCTCACTGAGGCCGGGCGACCAAAGGTCGCCCGACGCCCGGGCTTTG
CCCGGGCGGCCTCAGTGAGCGAGCGAGCGCGCAG
5' ITR

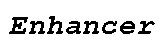

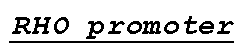

5' end portion of ABCA4

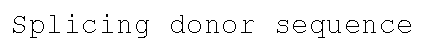

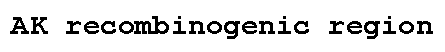

3' ITR

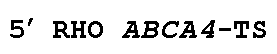

(SEQ ID NO: 27)
CTGCGCGCTCGCTCGCTCACTGAGGCCGCCCGGGCAAAGCCCGGGCGTCGGGCGACCTTTGG
TCGCCCGGCCTCAGTGAGCGAGCGAGCGCGCAGAGAGGGAGTGGCCAACTCCATCACTAGGG
GTTCCTTGTAGTTAATGATTAACCCGCCATGCTACTTATCTACGTAGCCATGCTCTAGGAAG

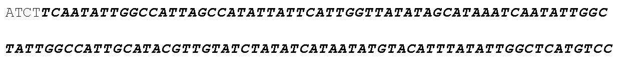

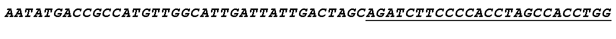

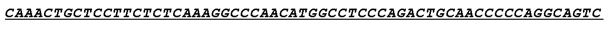

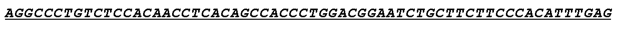

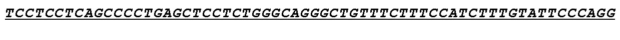

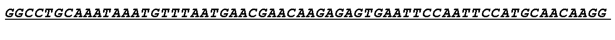

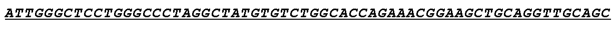

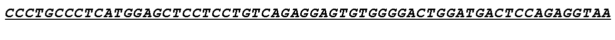

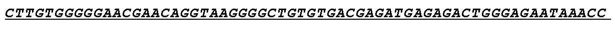

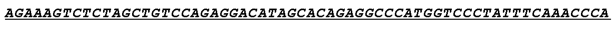

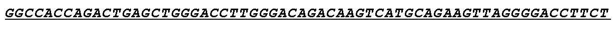

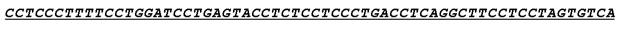

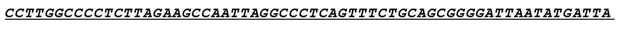

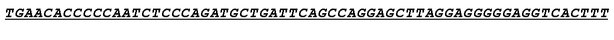

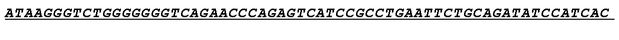

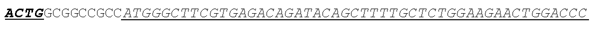

TGCGGAAAAGGCAAAAGATTCGCTTTGTGGTGGAACTCGTGTGGCCTTTATCTTTATTTCTG
GTCTTGATCTGGTTAAGGAATGCCAACCCGCTCTACAGCCATCATGAATGCCATTTCCCCAA
CAAGGCGATGCCCTCAGCAGGAATGCTGCCGTGGCTCCAGGGGATCTTCTGCAATGTGAACA
ATCCCTGTTTTCAAAGCCCCACCCCAGGAGAATCTCCTGGAATTGTGTCAAACTATAACAAC
TCCATCTTGGCAAGGGTATATCGAGATTTTCAAGAACTCCTCATGAATGCACCAGAGAGCCA
GCACCTTGGCCGTATTTGGACAGAGCTACACATCTTGTCCCAATTCATGGACACCCTCCGGA
CTCACCCGGAGAGAATTGCAGGAAGAGGAATTCGAATAAGGGATATCTTGAAAGATGAAGAA
ACACTGACACTATTTCTCATTAAAAACATCGGCCTGTCTGACTCAGTGGTCTACCTTCTGAT
CAACTCTCAAGTCCGTCCAGAGCAGTTCGCTCATGGAGTCCCGGACCTGGCGCTGAAGGACA
TCGCCTGCAGCGAGGCCCTCCTGGAGCGCTTCATCATCTTCAGCCAGAGACGCGGGGCAAAG
ACGGTGCGCTATGCCCTGTGCTCCCTCTCCCAGGGCACCCTACAGTGGATAGAAGACACTCT
GTATGCCAACGTGGACTTCTTCAAGCTCTTCCGTGTGCTTCCCACACTCCTAGACAGCCGTT
CTCAAGGTATCAATCTGAGATCTTGGGGAGGAATATTATCTGATATGTCACCAAGAATTCAA
GAGTTTATCCATCGGCCGAGTATGCAGGACTTGCTGTGGGTGACCAGGCCCCTCATGCAGAA
TGGTGGTCCAGAGACCTTTACAAAGCTGATGGGCATCCTGTCTGACCTCCTGTGTGGCTACC
CCGAGGGAGGTGGCTCTCGGGTGCTCTCCTTCAACTGGTATGAAGACAATAACTATAAGGCC
TTTCTGGGGATTGACTCCACAAGGAAGGATCCTATCTATTCTTATGACAGAAGAACAACATC
CTTTTGTAATGCATTGATCCAGAGCCTGGAGTCAAATCCTTTAACCAAAATCGCTTGGAGGG
CGGCAAAGCCTTTGCTGATGGGAAAAATCCTGTACACTCCTGATTCACCTGCAGCACGAAGG
ATACTGAAGAATGCCAACTCAACTTTTGAAGAACTGGAACACGTTAGGAAGTTGGTCAAAGC
CTGGGAAGAAGTAGGGCCCCAGATCTGGTACTTCTTTGACAACAGCACACAGATGAACATGA
TCAGAGATACCCTGGGGAACCCAACAGTAAAAGACTTTTTGAATAGGCAGCTTGGTGAAGAA
GGTATTACTGCTGAAGCCATCCTAAACTTCCTCTACAAGGGCCCTCGGGAAAGCCAGGCTGA
CGACATGGCCAACTTCGACTGGAGGGACATATTTAACATCACTGATCGCACCCTCCGCCTTG
TCAATCAATACCTGGAGTGCTTGGTCCTGGATAAGTTTGAAAGCTACAATGATGAAACTCAG
CTCACCCAACGTGCCCTCTCTCTACTGGAGGAAAACATGTTCTGGGCCGGAGTGGTATTCCC
TGACATGTATCCCTGGACCAGCTCTCTACCACCCCACGTGAAGTATAAGATCCGAATGGACA
TAGACGTGGTGGAGAAAACCAATAAGATTAAAGACAGGTATTGGGATTCTGGTCCCAGAGCT
GATCCCGTGGAAGATTTCCGGTACATCTGGGGCGGGTTTGCCTATCTGCAGGACATGGTTGA
ACAGGGGATCACAAGGAGCCAGGTGCAGGCGGAGGCTCCAGTTGGAATCTACCTCCAGCAGA
TGCCCTACCCCTGCTTCGTGGACGATTCTTTCATGATCATCCTGAACCGCTGTTTCCCTATC
TTCATGGTGCTGGCATGGATCTACTCTGTCTCCATGACTGTGAAGAGCATCGTCTTGGAGAA
GGAGTTGCGACTGAAGGAGACCTTGAAAAATCAGGGTGTCTCCAATGCAGTGATTTGGTGTA
CCTGGTTCCTGGACAGCTTCTCCATCATGTCGATGAGCATCTTCCTCCTGACGATATTCATC
ATGCATGGAAGAATCCTACATTACAGCGACCCATTCATCCTCTTCCTGTTCTTGTTGGCTTT
CTCCACTGCCACCATCATGCTGTGCTTTCTGCTCAGCACCTTCTTCTCCAAGGCCAGTCTGG
CAGCAGCCTGTAGTGGTGTCATCTATTTCACCCTCTACCTGCCACACATCCTGTGCTTCGCC
TGGCAGGACCGCATGACCGCTGAGCTGAAGAAGGCTGTGAGCTTACTGTCTCCGGTGGCATT
TGGATTTGGCACTGAGTACCTGGTTCGCTTTGAAGAGCAAGGCCTGGGGCTGCAGTGGAGCA
ACATCGGGAACAGTCCCACGGAAGGGGACGAATTCAGCTTCCTGCTGTCCATGCAGATGATG
CTCCTTGATGCTGCTGTCTATGGCTTACTCGCTTGGTACCTTGATCAGGTGTTTCCAGGAGA
CTATGGAACCCCACTTCCTTGGTACTTTCTTCTACAAGAGTCGTATTGGCTTGGCGGTGAAG
GGTGTTCAACCAGAGAAGAAAGAGCCCTGGAAAAGACCGAGCCCCTAACAGAGGAAACGGAG
GATCCAGAGCACCCAGAAGGAATACACGACTCCTTCTTTGAACGTGAGCATCCAGGGTGGGT
TCCTGGGGTATGCGTGAAGAATCTGGTAAAGATTTTTGAGCCCTGTGGCCGGCCAGCTGTGG
ACCGTCTGAACATCACCTTCTACGAGAACCAGATCACCGCATTCCTGGGCCACAATGGAGCT

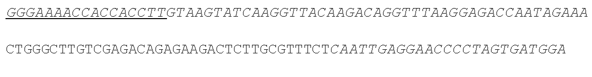

GTTGGCCACTCCCTCTCTGCGCGCTCGCTCGCTCACTGAGGCCGGGCGACCAAAGGTCGCCC
GACGCCCGGGCTTTGCCCGGGCGGCCTCAGTGAGCGAGCGAGCGCGCAG
5' ITR

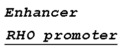

5' end portion of ABCA4

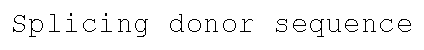

3' ITR

### Example 2. Dual AAV8.hMYO7A Response Study

Therapeutic efficacy of dual AAV8. MYO7A, including AAV8.5′MYO7A with the SV40 intron, was tested in vivo in shaker1^{-/-} (sh1^{-/-}) mice, which are a mouse model of Usher 1b. To select doses to be used in Usher syndrome type 1B (USH1B) subjects, we performed a dose response study using the dual AAV8.hMYO7A produced under good manufacturing-like practices (namely tox lot). Subretinally injected sh1^{-/-} mice, a mouse model of USH1B, were analyzed for rescue of retinal defects and protein hMYO7A levels. We selected three different doses: 1.37E+9 (low dose or LD), 4.4E+9 (medium dose or MD), and 1.37E+10 (high dose or HD) total GC/eye. Unaffected heterozygous mice and affected mice injected with the AAV solvent only (phosphate buffered saline supplemented with NaCl 35 mM and 0.001% Poloxamer 188) were used as positive and negative controls, respectively. Sh1^{-/-} mice display ultrastructural defects of the retina, as almost no melanosomes are located to the retinal pigment epithelium (RPE) apical villi. Three months post-injection, we confirmed the dose-dependent effects by measuring the number of correctly localized melanosomes to the RPE apical villi (FIG. 5A-B). Injection of HD and MD of dual AAV8.hMYO7A significantly rescued retinal defects compared to sh1^{-/-} that only received the solvent; moreover, there was no statistical difference between unaffected eyes and affected eyes treated with HD (FIG. 5B, pANOVA values: affected sh1^{-/-} injected with formulation buffer vs either unaffected sh1^{+/-} injected with formulation buffer <0,0001, sh1^{-/-} treated with the high dose <0,0001, treated with the medium dose <0.01 or sh1^{-/-} treated with the low dose=0,313; sh1^{-/-} treated with the high dose vs either unaffected sh1^{+/-} injected with formulation buffer=0,105, treated with the medium dose=0,113 or sh1^{-/-} treated with the low dose <0.01; sh1^{-/-} treated with the medium dose vs either unaffected sh1^{+/-} injected with formulation buffer <0,001 or treated with the low dose=0,442; unaffected sh1^{+/-} injected with formulation buffer vs sh1^{-/-} treated with the low dose <0,0001). Sh1^{-/-} LD-treated eyes also showed correction of the retinal phenotype compared to the negative control. There was some variability within the unaffected sh1^{+/-} group that affected statistical analysis, thus we repeated the ANOVA analysis without unaffected sh1^{+/-} and reached statistical significance for the LD as well (FIG. 5B, pANOVA values: affected sh1^{-/-} injected with formulation buffer vs sh1^{-/-} treated with the high dose <0,0001, sh1^{-/-} treated with the medium dose <0,0001 or sh1^{-/-} treated with the low dose <0.01; sh1^{-/-} treated with the high dose vs either sh1^{-/-} treated with the medium dose <0,001 or sh1^{-/-} treated with the low dose <0,0001; sh1^{-/-} treated with the medium dose vs treated with the low dose <0.05). Western blot analysis of lysed eyecups (RPE+neural retina) from sh1^{-/-} mice 5 weeks after sub-retinal injection displays expression of the full length hMYO7A for all selected doses of dual AAV8.hMYO7A (FIG. 5C-D). A higher number of eyes were positive for hMYO7A expression using the HD and the MD compared to the LD (FIG. 5D). Considering that human retina is 100× the murine retina (Panda-Jonas et al. (1994) Ophthalmology 101: 519-523; Remtulla et al. (1985) Vision Res. 25: 21-31), we can infer that corresponding therapeutics doses in humans may range between 1.37E+11 and 1.37E+12 total GC/eye of dual AAV8.hMYO7A.

Materials and Methods

Western Blot Analysis

Eyecups (cups+retinas) for Western blot (WB) analysis were lysed in RIPA buffer (50 mM Tris-HCl pH 8.0, 150 mM NaCl, 1% NP40, 0.5% Na-Deoxycholate, 1 mM EDTA pH 8.0, 0.1% SDS). Lysis buffer was supplemented with 0.5% phenylmethylsulfonyl fluoride (PSMF) (Sigma-Aldrich, St. Louis, Mo.) and 1% complete EDTA-free protease inhibitor cocktail (Roche, Milan, Italy). Protein concentration was determined using Pierce BCA protein assay kit (Thermo-Scientific, Waltham, Mass.). After lysis, samples were denatured at 99° C. for 5 min in 4× Laemmli sample buffer (Bio-rad, Milan, Italy) supplemented with 11-mercaptoethanol (Sigma-Aldrich) diluted 1:10. Samples for MYO7A analysis on 4-20% gradient pre-cast TGX gels (Bio-rad). The following antibodies were used for immuno-blotting: custom anti-hMYO7A (1:200, polyclonal; Primm Srl, Milan, Italy) that recognizes a peptide corresponding to amino acids 941-1070 of the hMYO7A protein (DMVDKMFGFLGTSGGLPGQEGQAPSGFEDLERGRREMVEEDLDAALPLPDEDEEDLSEY KFAKFAATYFQGTTTHSYTRRPLKQPLLYHDDEGDQLAALAVWITILRFMGDLPEPKYHTAM SDGSEKIPV; underlined aminoacids are different (1.6%) in murine Myo7A); anti-Dysferlin (1:500, MONX10795; Tebu-bio, Le Perray-en-Yveline, France). The quantification of WB bands was performed using ImageJ software. hMYO7A expression was normalized over the expression of Dysferlin.

Melanosome Localization Analysis

Eyes from pigmented sh1 mice (+/− or −/−) were enucleated 3 months following the AAV injection and cauterized on the temporal side of the cornea. Fixation was performed using 2% glutaraldehyde-2% paraformaldehyde in 0.1 M PBS overnight, rinsed in 0.1 M PBS and dissected under a light microscope. The temporal portions of the eyecups were embedded in Araldite 502/EMbed 812 (Araldite 502/EMbed 812 KIT, catalog #13940; Electron Microscopy Sciences, Hatfield, Pa., USA). Semi-thin (0.5 μm) sections were transversally cut on a Leica Ultramicrotome RM2235 (Leica Microsystems, Bannockburn, Ill., USA), mounted on slides and stained with toluidine blue and borace staining. Melanosomes were counted by a masked operator in a montage of the entire retinal section obtained through acquisition of overlapping fields using a Zeiss Apotome (Carl Zeiss, Oberkochen, Germany) with 100× magnification; then, the entire retinal section was reconstituted on Photoshop software (Adobe, San Jose, Calif.). Melanosomes count and retinal pigment epithelium (RPE) measurements were performed using ImageJ software. Melanosome number was normalized over the length of the RPE divided by 100 μm.

Statistical Analysis

One-way analysis of variance (ANOVA) followed by Tuckey post-hoc analysis was used to perform multi pairwise comparisons between groups in FIG. 5. FIG. 5: dose-dependent effects on correctly localized melanosomes to the retinal pigment epithelium: the ANOVA p-values are the following. Affected sh1^{-/-} injected with formulation buffer Vs either unaffected sh1^{+/-} injected with formulation buffer (pANOVA <0,0001), sh1^{-/-} treated with the high dose (pANOVA <0,0001), sh1^{-/-} treated with the medium dose (pANOVA <0.01) or sh1^{-/-} treated with the low dose (pANOVA=0,313); sh1^{-/-} treated with the high dose Vs either unaffected sh1^{+/-} injected with formulation buffer (pANOVA=0,105), sh1^{-/-} treated with the medium dose (pANOVA=0,113) or sh1^{-/-} treated with the low dose (pANOVA <0.01); sh1^{-/-} treated with the medium dose Vs either unaffected sh1^{+/-} injected with formulation buffer (pANOVA <0,001) or sh1^{-/-} treated with the low dose (pANOVA 0,442); unaffected sh1^{+/-} injected with formulation buffer Vs sh1^{-/-} treated with the low dose (pANOVA <0,0001). Due to the variability of sh1^{+/-} injected with formulation buffer impacting the ANOVA analysis, comparisons were analyzed again without unaffected controls and the ANOVA p-values are the following: affected sh1^{-/-} injected with formulation buffer Vs sh1^{-/-} treated with the high dose (pANOVA <0,0001), sh1^{-/-} treated with the medium dose (pANOVA <0,0001) or sh1^{-/-} treated with the low dose (pANOVA <0.01); sh1^{-/-} treated with the high dose Vs either sh1^{-/-} treated with the medium dose (pANOVA <0.001) or sh1^{-/-} treated with the low dose (pANOVA <0,0001); sh1^{-/-} treated with the medium dose Vs sh1^{-/-} treated with the low dose (pANOVA <0.05). Data are presented as mean [±standard error of the mean (s.e.m.)] which has been calculated using the number of independent in vitro experiments or eyes (not replicate measurements of the same sample). Statistical p-values ≤0.05 were considered significant.

All publications mentioned in the above specification are herein incorporated by reference. Various modifications and variations of the disclosed vectors, systems, methods or uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1. A vector system for expressing a transgene in a cell, the vector system comprising a first vector and a second vector, wherein:
(a) the first vector comprises in a 5′ to 3′ direction: a promoter; an intron; a 5′ end portion of the transgene coding sequence (CDS); a splice donor sequence; and a first recombinogenic region;
(b) the second vector comprises in a 5′ to 3′ direction: a second recombinogenic region; a splice acceptor sequence; and a 3′ end portion of the transgene CDS;
wherein the 5′ end portion and the 3′ end portion together constitute the transgene CDS, and wherein the intron is not capable of homologous recombination with the splice donor sequence to excise the 5′ end portion of the transgene CDS.

2. The vector system of claim 1, wherein the intron does not comprise a region of at least 20, 30, 40, 50, 60, 70, 80, 90 or 100 contiguous nucleotides having at least 95%, 96%, 97%, 98%, 99% or 100% (preferably 100%) sequence identity to a region of the splice donor sequence.

3. The vector system of claim 1, wherein the intron:
(a) is a simian virus 40 (SV40) intron or a minute virus mice (MVM) intron; and/or
(b) comprises a nucleotide sequence with at least 95% sequence identity to SEQ ID NO: 3 or 4.

4. The vector system of claim 1, wherein the splice donor sequence comprises a nucleotide sequence with at least 95% sequence identity to SEQ ID NO: 5.

5. The vector system of claim 1, wherein the first recombinogenic region and the second recombinogenic region:
(a) are both F1 phage recombinogenic regions or fragments thereof; and/or
(b) both comprise a nucleotide sequence with at least 95% sequence identity to SEQ ID NO: 7 or a fragment thereof.

6. The vector system of claim 1, wherein the first vector and the second vector are viral vectors.

7. The vector system of claim 1, wherein the first vector and the second vector are AAV vectors.

8. The vector system of claim 1, wherein the promoter is a CBA promoter or a fragment thereof.

9. The vector system of claim 1, wherein the second vector further comprises a polyadenylation sequence downstream of the 3′ end portion of the transgene CDS.

10. The vector system of claim 1, wherein the transgene is a Myosin 7A (MYO7A) transgene.

11. The vector system of claim 1, wherein:
(a) the first vector comprises a nucleotide sequence with at least 95% sequence identity to SEQ ID NO: 14; and/or
(b) the second vector comprises a nucleotide sequence with at least 95% sequence identity to SEQ ID NO: 15.

12. A method for expressing a transgene in a cell, comprising transducing or transfecting the cell with the first vector and the second vector as defined in claim 1, such that the transgene is expressed in the cell.

13. A vector comprising in a 5′ to 3′ direction: a promoter; an intron; a 5′ end portion of a transgene coding sequence (CDS); a splice donor sequence; and a recombinogenic region, wherein the intron is not capable of homologous recombination with the splice donor sequence to excise the 5′ end portion of the transgene CDS.

14. The vector of claim 13, wherein the vector comprises a nucleotide sequence with at least 95% sequence identity to SEQ ID NO: 14.

15. (canceled)

16. (canceled)

17. A method of treating or preventing Usher syndrome comprising administering an effective amount of the vector system of claim 1 to a subject in need thereof.

18. The method of claim 17, wherein the Usher syndrome is Usher syndrome Type 1B.

19. The vector system of claim 7, wherein the first vector further comprises a 5′ ITR and a 3′ ITR, and the second vector further comprises a 5′ ITR and a 3′ ITR.
